SEQUENCE LISTING (194 sequences)

SEQ ID NO: 1 - protein, 67 aa, Chlamydia trachomatis
MQHTIMLSLENDNDKLASMMDRVVAASSSILSASKDSESNRQFTISKAPDKEAPCRVSYVAASALSE

SEQ ID NO: 2 - DNA, 204 bp, Chlamydia trachomatis
atgcaacacacaattatgctgtctttagagaacgataatgataagcttgcttctatgatggatcgagttgttgctgcgtcatcaagcattctttctgcttccaaagattctgagtccaatagacagtttactatttctaaagctccggataaagaagctccttgcagagtatcttatgtagctgcaagtgcactttcagaatag

SEQ ID NO: 3 - protein, 9 aa, Chlamydia trachomatis
  BINDING (1)..(9): CT263 immunogenic peptide
KLAEAIFPI

SEQ ID NO: 4 - protein, 9 aa, Chlamydia trachomatis
  BINDING (1)..(9): CT263 immunogenic peptide
FLKNNKVKL

SEQ ID NO: 5 - protein, 9 aa, Chlamydia trachomatis
  BINDING (1)..(9): CT263 immunogenic peptide
ALSPPPSGY

SEQ ID NO: 6 - protein, 9 aa, Chlamydia trachomatis
  BINDING (1)..(9): CT263 immunogenic peptide
FIAKQASLV

SEQ ID NO: 7 - protein, 9 aa, Chlamydia trachomatis
  BINDING (1)..(9): CT263 immunogenic peptide
TLSLFPFSL

SEQ ID NO: 8 - protein, 9 aa, Chlamydia trachomatis
  BINDING (1)..(9): CT263 immunogenic peptide
SLVACPCSM

SEQ ID NO: 9 - protein, 9 aa, Chlamydia trachomatis
  BINDING (1)..(9): CT263 immunogenic peptide
LIFADPAEA

SEQ ID NO: 10 - protein, 9 aa, Chlamydia trachomatis
  BINDING (1)..(9): CT263 immunogenic peptide
LLLIFADPA

SEQ ID NO: 11 - protein, 9 aa, Chlamydia trachomatis
  BINDING (1)..(9): CT541 immunogenic peptide
ILSWMLMFA

SEQ ID NO: 12 - protein, 9 aa, Chlamydia trachomatis
  BINDING (1)..(9): CT541 immunogenic peptide
KQMAEVQKA

SEQ ID NO: 13 - protein, 9 aa, Chlamydia trachomatis
  BINDING (1)..(9): CT541 immunogenic peptide
LMFAVALPI

SEQ ID NO: 14 - protein, 9 aa, Chlamydia trachomatis
  BINDING (1)..(9): CT541 immunogenic peptide
KLQYRVVKE

SEQ ID NO: 15 - protein, 9 aa, Chlamydia trachomatis
  BINDING (1)..(9): CT541 immunogenic peptide
FLKENKEKA

SEQ ID NO: 16 - protein, 9 aa, Chlamydia trachomatis
  BINDING (1)..(9): CT541 immunogenic peptide
KLSRTFGHL

SEQ ID NO: 17 - protein, 9 aa, Chlamydia trachomatis
  BINDING (1)..(9): CT541 immunogenic peptide
SLLIFEVKL

SEQ ID NO: 18 - protein, 9 aa, Chlamydia trachomatis
  BINDING (1)..(9): CT541 immunogenic peptide
VLSGKPTAL

SEQ ID NO: 19 - protein, 9 aa, Chlamydia trachomatis
  BINDING (1)..(9): CT541 immunogenic peptide
VLYIHPDLA

SEQ ID NO: 20 - protein, 9 aa, Chlamydia trachomatis
  BINDING (1)..(9): CT541 immunogenic peptide
LLSRQLSRT

SEQ ID NO: 21 - protein, 9 aa, Chlamydia trachomatis
  BINDING (1)..(9): CT691 immunogenic peptide
LLQRELMKV

SEQ ID NO: 22 - protein, 9 aa, Chlamydia trachomatis
  BINDING (1)..(9): CT691 immunogenic peptide
STINVLFPL

SEQ ID NO: 23 - protein, 9 aa, Chlamydia trachomatis
  BINDING (1)..(9): CT691 immunogenic peptide
PLQAHLELV

SEQ ID NO: 24 - protein, 9 aa, Chlamydia trachomatis
  BINDING (1)..(9): CT691 immunogenic peptide
SLFGQSPFA

SEQ ID NO: 25 - protein, 9 aa, Chlamydia trachomatis
  BINDING (1)..(9): CT691 immunogenic peptide
KLAYRVSMT

SEQ ID NO: 26 - protein, 9 aa, Chlamydia trachomatis
  BINDING (1)..(9): CT691 immunogenic peptide
VLWMQIIKG

SEQ ID NO: 27 - protein, 9 aa, Chlamydia trachomatis
  BINDING (1)..(9): CT691 immunogenic peptide
VLFPLFSAL

SEQ ID NO: 28 - protein, 9 aa, Chlamydia trachomatis
  BINDING (1)..(9): CT691 immunogenic peptide
FLQKTVQSF

SEQ ID NO: 29 - protein, 9 aa, Chlamydia trachomatis
  BINDING (1)..(9): CT691 immunogenic peptide
FGQSPFAPL

SEQ ID NO: 30 - protein, 9 aa, Chlamydia trachomatis
  BINDING (1)..(9): CT858 immunogenic peptide
VLADFIGGL

SEQ ID NO: 31 - protein, 9 aa, Chlamydia trachomatis
  BINDING (1)..(9): CT858 immunogenic peptide
RMASLGHKV

SEQ ID NO: 32 - protein, 9 aa, Chlamydia trachomatis
  BINDING (1)..(9): CT858 immunogenic peptide
GLNDFHAGV

SEQ ID NO: 33 - protein, 9 aa, Chlamydia trachomatis
  BINDING (1)..(9): CT858 immunogenic peptide
FSCADFFPV

SEQ ID NO: 34 - protein, 9 aa, Chlamydia trachomatis
  BINDING (1)..(9): CT858 immunogenic peptide
MLTDRPLEL

SEQ ID NO: 35 - protein, 9 aa, Chlamydia trachomatis
  BINDING (1)..(9): CT858 immunogenic peptide
LLENVDTNV

SEQ ID NO: 36 - protein, 9 aa, Chlamydia trachomatis
  BINDING (1)..(9): CT858 immunogenic peptide
RMILTQDEV

SEQ ID NO: 37 - protein, 9 aa, Chlamydia trachomatis
  BINDING (1)..(9): CT858 immunogenic peptide
SCADFFPVV

SEQ ID NO: 38 - protein, 9 aa, Chlamydia trachomatis
  BINDING (1)..(9): CT858 immunogenic peptide
FVFNVQFPN

SEQ ID NO: 39 - protein, 9 aa, Chlamydia trachomatis
  BINDING (1)..(9): CT858 immunogenic peptide
YLYALLSML

SEQ ID NO: 40 - protein, 9 aa, Chlamydia trachomatis
  BINDING (1)..(9): CT858 immunogenic peptide
SLAVREHGA

SEQ ID NO: 41 - protein, 9 aa, Chlamydia trachomatis
  BINDING (1)..(9): CT858 immunogenic peptide
YLPYTVQKS

SEQ ID NO: 42 - protein, 9 aa, Chlamydia trachomatis
  BINDING (1)..(9): CT858 immunogenic peptide
ATIAPSIRA

SEQ ID NO: 43 - protein, 9 aa, Chlamydia trachomatis
  BINDING (1)..(9): CT858 immunogenic peptide
LLEVDGAPV

SEQ ID NO: 44 - protein, 9 aa, Chlamydia trachomatis
  BINDING (1)..(9): CT858 immunogenic peptide
RTAGAGGFV

SEQ ID NO: 45 - protein, 9 aa, Chlamydia trachomatis
  BINDING (1)..(9): CT858 immunogenic peptide
SLFYSPMVP

SEQ ID NO: 46 - protein, 9 aa, Chlamydia pneumoniae
  BINDING (1)..(9): CPN0152  immunogenic peptide
FLVSCLFSV

SEQ ID NO: 47 - protein, 9 aa, Chlamydia pneumoniae
  BINDING (1)..(9): CPN0152  immunogenic peptide
YLRDAQTIL

SEQ ID NO: 48 - protein, 9 aa, Chlamydia pneumoniae
  BINDING (1)..(9): CPN0152  immunogenic peptide
LLIRIQDHV

SEQ ID NO: 49 - protein, 9 aa, Chlamydia pneumoniae
  BINDING (1)..(9): CPN0152  immunogenic peptide
KLGRKFAAV

SEQ ID NO: 50 - protein, 9 aa, Chlamydia pneumoniae
  BINDING (1)..(9): CPN0152  immunogenic peptide
LVSRTQQTL

SEQ ID NO: 51 - protein, 9 aa, Chlamydia pneumoniae
  BINDING (1)..(9): CPN0152  immunogenic peptide
CLFSVAIGA

SEQ ID NO: 52 - protein, 9 aa, Chlamydia pneumoniae
  BINDING (1)..(9): CPN0152  immunogenic peptide
GFGPPPIIV

SEQ ID NO: 53 - protein, 9 aa, Chlamydia pneumoniae
  BINDING (1)..(9): CPN0152  immunogenic peptide
SLPTKPYIL

SEQ ID NO: 54 - protein, 9 aa, Chlamydia pneumoniae
  BINDING (1)..(9): CPN0152  immunogenic peptide
RIQDHVTAN

SEQ ID NO: 55 - protein, 9 aa, Chlamydia pneumoniae
  BINDING (1)..(9): CPN0152  immunogenic peptide
AIGASAAPV

SEQ ID NO: 56 - protein, 9 aa, Chlamydia pneumoniae
  BINDING (1)..(9): CPN0152  immunogenic peptide
RLGISGFSL

SEQ ID NO: 57 - protein, 9 aa, Chlamydia pneumoniae
  BINDING (1)..(9): CPN0619  immunogenic peptide
FMVSGPVVV

SEQ ID NO: 58 - protein, 9 aa, Chlamydia pneumoniae
  BINDING (1)..(9): CPN0619  immunogenic peptide
LVLEGANAV

SEQ ID NO: 59 - protein, 9 aa, Chlamydia pneumoniae
  BINDING (1)..(9): CPN0705  immunogenic peptide
FVGANLTLV

SEQ ID NO: 60 - protein, 9 aa, Chlamydia pneumoniae
  BINDING (1)..(9): CPN0619  immunogenic peptide
CLAENAFAG

SEQ ID NO: 61 - protein, 9 aa, Chlamydia pneumoniae
  BINDING (1)..(9): CPN0705  immunogenic peptide
KIEEVQTPL

SEQ ID NO: 62 - protein, 9 aa, Chlamydia pneumoniae
  BINDING (1)..(9): CPN0705  immunogenic peptide
ALKGHQLTL

SEQ ID NO: 63 - protein, 9 aa, Chlamydia pneumoniae
  BINDING (1)..(9): CPN0705  immunogenic peptide
QMAEAADLV

SEQ ID NO: 64 - protein, 9 aa, Chlamydia pneumoniae
  BINDING (1)..(9): CPN0796  immunogenic peptide
FMGAHVFAS

SEQ ID NO: 65 - protein, 9 aa, Chlamydia pneumoniae
  BINDING (1)..(9): CPN0796  immunogenic peptide
LLIQHSAKV

SEQ ID NO: 66 - protein, 9 aa, Chlamydia pneumoniae
  BINDING (1)..(9): CPN0796  immunogenic peptide
FLCPFQAPS

SEQ ID NO: 67 - protein, 9 aa, Chlamydia pneumoniae
  BINDING (1)..(9): CPN0796  immunogenic peptide
FQAPSPAPV

SEQ ID NO: 68 - protein, 9 aa, Chlamydia pneumoniae
  BINDING (1)..(9): CPN0796  immunogenic peptide
AMNACVNGI

SEQ ID NO: 69 - protein, 9 aa, Chlamydia pneumoniae
  BINDING (1)..(9): CPN0796  immunogenic peptide
FMGIQVLHL

SEQ ID NO: 70 - protein, 9 aa, Chlamydia pneumoniae
  BINDING (1)..(9): CPN0796  immunogenic peptide
RHAAQATGV

SEQ ID NO: 71 - protein, 9 aa, Chlamydia pneumoniae
  BINDING (1)..(9): CPN0796  immunogenic peptide
FQYADGQMV

SEQ ID NO: 72 - protein, 9 aa, Chlamydia pneumoniae
  BINDING (1)..(9): CPN0796  immunogenic peptide
SVSAMGNFV

SEQ ID NO: 73 - protein, 9 aa, Chlamydia pneumoniae
  BINDING (1)..(9): CPN0796  immunogenic peptide
FLSYRSQVH

SEQ ID NO: 74 - protein, 9 aa, Chlamydia pneumoniae
  BINDING (1)..(9): CPN0796  immunogenic peptide
FLLTAIPGS

SEQ ID NO: 75 - protein, 9 aa, Chlamydia pneumoniae
  BINDING (1)..(9): CPN0796  immunogenic peptide
VLTPWIYRK

SEQ ID NO: 76 - protein, 9 aa, Chlamydia pneumoniae
  BINDING (1)..(9): CPN0796  immunogenic peptide
SVVMNQQPL

SEQ ID NO: 77 - protein, 9 aa, Chlamydia pneumoniae
  BINDING (1)..(9): CPN0796  immunogenic peptide
SLKNSQQQL

SEQ ID NO: 78 - protein, 9 aa, Chlamydia pneumoniae
  BINDING (1)..(9): CPN0796  immunogenic peptide
MLPDTLDSV

SEQ ID NO: 79 - protein, 9 aa, Chlamydia pneumoniae
  BINDING (1)..(9): CPN0796  immunogenic peptide
ALPYTEQGL

SEQ ID NO: 80 - protein, 9 aa, Chlamydia pneumoniae
  BINDING (1)..(9): CPN0796  immunogenic peptide
VLSGFGGQV

SEQ ID NO: 81 - protein, 9 aa, Chlamydia pneumoniae
  BINDING (1)..(9): CPN0998  immunogenic peptide
LLFGVVFGV

SEQ ID NO: 82 - protein, 9 aa, Chlamydia pneumoniae
  BINDING (1)..(9): CPN0998  immunogenic peptide
SLQERYPTL

SEQ ID NO: 83 - protein, 9 aa, Chlamydia pneumoniae
  BINDING (1)..(9): CPN0998  immunogenic peptide
MLLKGQNKV

SEQ ID NO: 84 - protein, 9 aa, Chlamydia pneumoniae
  BINDING (1)..(9): CPN0998  immunogenic peptide
FTFLPIILV

SEQ ID NO: 85 - protein, 9 aa, Chlamydia pneumoniae
  BINDING (1)..(9): CPN0998  immunogenic peptide
FLGDISSGA

SEQ ID NO: 86 - protein, 9 aa, Chlamydia pneumoniae
  BINDING (1)..(9): CPN0998  immunogenic peptide
FLAGKKARV

SEQ ID NO: 87 - protein, 9 aa, Chlamydia pneumoniae
  BINDING (1)..(9): CPN0998  immunogenic peptide
LLDAAYQRA

SEQ ID NO: 88 - protein, 9 aa, Chlamydia pneumoniae
  BINDING (1)..(9): CPN0998  immunogenic peptide
YLGYLFTFL

SEQ ID NO: 89 - protein, 9 aa, Chlamydia pneumoniae
  BINDING (1)..(9): CPN0998 immunogenic peptide
YLFTFLPII

SEQ ID NO: 90 - protein, 9 aa, Chlamydia pneumoniae
  BINDING (1)..(9): CPN0998 immunogenic peptide
SLGATHFLP

SEQ ID NO: 91 - protein, 9 aa, Chlamydia pneumoniae
  BINDING (1)..(9): CPN0998 immunogenic peptide
ELIDQGHRL

SEQ ID NO: 92 - protein, 9 aa, Chlamydia pneumoniae
  BINDING (1)..(9): CPN0998 immunogenic peptide
FLPIILVLL

SEQ ID NO: 93 - protein, 9 aa, Chlamydia pneumoniae
  BINDING (1)..(9): CPN0998 immunogenic peptide
LVLLFVYLV

SEQ ID NO: 94 - protein, 9 aa, Chlamydia pneumoniae
  BINDING (1)..(9): CPN0998 immunogenic peptide
VTGPATPQL

SEQ ID NO: 95 - protein, 9 aa, Chlamydia pneumoniae
  BINDING (1)..(9): CPN0998 immunogenic peptide
SLEKQDPEV

SEQ ID NO: 96 - protein, 9 aa, Chlamydia pneumoniae
  BINDING (1)..(9): CPN0998 immunogenic peptide
ILMAATNRP

SEQ ID NO: 97 - protein, 9 aa, Chlamydia pneumoniae
  BINDING (1)..(9): CPN0998 immunogenic peptide
LTQETDTEA

SEQ ID NO: 98 - protein, 9 aa, Chlamydia pneumoniae
  BINDING (1)..(9): CPN0998 immunogenic peptide
MLLDAAYQR

SEQ ID NO: 99 - protein, 9 aa, Chlamydia pneumoniae
  BINDING (1)..(9): CPN0998 immunogenic peptide
LLNEAALLA

SEQ ID NO: 100 - protein, 9 aa, Chlamydia pneumoniae
  BINDING (1)..(9): CPN0998 immunogenic peptide
ELYDQLAVL

SEQ ID NO: 101 - protein, 9 aa, Chlamydia pneumoniae
  BINDING (1)..(9): CPN0998 immunogenic peptide
LIGKYLSPV

SEQ ID NO: 102 - protein, 9 aa, Chlamydia pneumoniae
  BINDING (1)..(9): CPN0998 immunogenic peptide
SLGGRIPKG

SEQ ID NO: 103 - protein, 9 aa, Chlamydia pneumoniae
  BINDING (1)..(9): CPN0998 immunogenic peptide
GMSPQLGNV

SEQ ID NO: 104 - protein, 9 aa, Chlamydia pneumoniae
  BINDING (1)..(9): CPN0998 immunogenic peptide
LLAARKDRT

SEQ ID NO: 105 - protein, 9 aa, Chlamydia pneumoniae
  BINDING (1)..(9): CPN0998 immunogenic peptide
VTFADVAGI

SEQ ID NO: 106 - protein, 9 aa, Chlamydia pneumoniae
  BINDING (1)..(9): CPN0998 immunogenic peptide
VLTEPLVVT

SEQ ID NO: 107 - protein, 9 aa, Chlamydia pneumoniae
  BINDING (1)..(9): CPN0998 immunogenic peptide
KIALNDNLV

SEQ ID NO: 108 - protein, 9 aa, Chlamydia pneumoniae
  BINDING (1)..(9): CPN1016  immunogenic peptide
KLGAIVFGL

SEQ ID NO: 109 - protein, 9 aa, Chlamydia pneumoniae
  BINDING (1)..(9): CPN1016  immunogenic peptide
YLGDEILEV

SEQ ID NO: 110 - protein, 9 aa, Chlamydia pneumoniae
  BINDING (1)..(9): CPN1016  immunogenic peptide
FLPTFGPIL

SEQ ID NO: 111 - protein, 9 aa, Chlamydia pneumoniae
  BINDING (1)..(9): CPN1016  immunogenic peptide
SLQNFSQSV

SEQ ID NO: 112 - protein, 9 aa, Chlamydia pneumoniae
  BINDING (1)..(9): CPN1016  immunogenic peptide
FTDEQAVAV

SEQ ID NO: 113 - protein, 9 aa, Chlamydia pneumoniae
  BINDING (1)..(9): CPN1016  immunogenic peptide
SLNDYHAGI

SEQ ID NO: 114 - protein, 9 aa, Chlamydia pneumoniae
  BINDING (1)..(9): CPN1016  immunogenic peptide
RMIFTQDEV

SEQ ID NO: 115 - protein, 9 aa, Chlamydia pneumoniae
  BINDING (1)..(9): CPN1016  immunogenic peptide
TQQARLQLV

SEQ ID NO: 116 - protein, 9 aa, Chlamydia pneumoniae
  BINDING (1)..(9): CPN1016  immunogenic peptide
SLVAPLIPE

SEQ ID NO: 117 - protein, 9 aa, Chlamydia pneumoniae
  BINDING (1)..(9): CPN1016  immunogenic peptide
YMVPYFWEE

SEQ ID NO: 118 - protein, 9 aa, Chlamydia pneumoniae
  BINDING (1)..(9): CPN1016  immunogenic peptide
YVEAVKTIV

SEQ ID NO: 119 - protein, 9 aa, Chlamydia pneumoniae
  BINDING (1)..(9): CPN1016  immunogenic peptide
FTQDEVSSA

SEQ ID NO: 120 - protein, 9 aa, Chlamydia pneumoniae
  BINDING (1)..(9): CPN1016  immunogenic peptide
GAGGFVFQV

SEQ ID NO: 121 - protein, 9 aa, Chlamydia pneumoniae
  BINDING (1)..(9): CPN1016  immunogenic peptide
LLGFAQVRP

SEQ ID NO: 122 - protein, 9 aa, Chlamydia pneumoniae
  BINDING (1)..(9): CPN0412 immunogenic peptide
RLEEVSQKL

SEQ ID NO: 123 - protein, 9 aa, Chlamydia pneumoniae
  BINDING (1)..(9): CPN0412 immunogenic peptide
LTTDTPPVL

SEQ ID NO: 124 - protein, 9 aa, Chlamydia pneumoniae
  BINDING (1)..(9): CPN0412 immunogenic peptide
KLLDMEGYA

SEQ ID NO: 125 - protein, 9 aa, Chlamydia pneumoniae
  BINDING (1)..(9): CPN0412 immunogenic peptide
VLSEDPPYI

SEQ ID NO: 126 - protein, 9 aa, Chlamydia pneumoniae
  BINDING (1)..(9): CPN0412 immunogenic peptide
ALQSYCQAY

SEQ ID NO: 127 - protein, 9 aa, Chlamydia pneumoniae
  BINDING (1)..(9): CPN0412 immunogenic peptide
KLTQTLVEL

SEQ ID NO: 128 - protein, 9 aa, Chlamydia pneumoniae
  BINDING (1)..(9): CPN0412 immunogenic peptide
FVGACSPEI

SEQ ID NO: 129 - protein, 9 aa, Chlamydia pneumoniae
  BINDING (1)..(9): CPN0412 immunogenic peptide
NLTTDTPPV

SEQ ID NO: 130 - protein, 9 aa, Chlamydia pneumoniae
  BINDING (1)..(9): CPN0412 immunogenic peptide
LMERAIPPK

SEQ ID NO: 131 - protein, 9 aa, Chlamydia pneumoniae
  BINDING (1)..(9): CPN0661 immunogenic peptide
KMAEVQKLV

SEQ ID NO: 132 - protein, 9 aa, Chlamydia pneumoniae
  BINDING (1)..(9): CPN0661 immunogenic peptide
ALGMQGMKE

SEQ ID NO: 133 - protein, 9 aa, Chlamydia pneumoniae
  BINDING (1)..(9): CPN0661 immunogenic peptide
KLSRTFGHL

SEQ ID NO: 134 - protein, 9 aa, Chlamydia pneumoniae
  BINDING (1)..(9): CPN0661 immunogenic peptide
LLIFEINLI

SEQ ID NO: 135 - protein, 9 aa, Chlamydia pneumoniae
  BINDING (1)..(9): CPN0661 immunogenic peptide
VLATVALAL

SEQ ID NO: 136 - protein, 9 aa, Chlamydia pneumoniae
  BINDING (1)..(9): CPN0661 immunogenic peptide
ILLPLGQTI

SEQ ID NO: 137 - protein, 9 aa, Chlamydia pneumoniae
  BINDING (1)..(9): CPN0661 immunogenic peptide
VLYIHPDLA

SEQ ID NO: 138 - protein, 9 aa, Chlamydia pneumoniae
  HOMOLOGY (1)..(9): Homolog to CT541 immunogenic peptide
LVLATVALA

SEQ ID NO: 139 - protein, 9 aa, Chlamydia pneumoniae
  BINDING (1)..(9): CPN0681 immunogenic peptide
YMLPIFTAL

SEQ ID NO: 140 - protein, 9 aa, Chlamydia pneumoniae
  BINDING (1)..(9): CPN0681 immunogenic peptide
LLHEFNQLL

SEQ ID NO: 141 - protein, 9 aa, Chlamydia pneumoniae
  BINDING (1)..(9): CPN0681 immunogenic peptide
VLQRELMQI

SEQ ID NO: 142 - protein, 9 aa, Chlamydia pneumoniae
  BINDING (1)..(9): CPN0681 immunogenic peptide
PLQAHLEMV

SEQ ID NO: 143 - protein, 9 aa, Chlamydia pneumoniae
  BINDING (1)..(9): CPN0681 immunogenic peptide
RLFGQSPFA

SEQ ID NO: 144 - protein, 9 aa, Chlamydia pneumoniae
  BINDING (1)..(9): CPN0681 immunogenic peptide
GLFMPISRA

SEQ ID NO: 145 - protein, 9 aa, Chlamydia pneumoniae
  BINDING (1)..(9): CPN0681 immunogenic peptide
KLAHRINMT

SEQ ID NO: 146 - protein, 9 aa, Chlamydia pneumoniae
  BINDING (1)..(9): CPN0681 immunogenic peptide
YLWLQVIRR

SEQ ID NO: 147 - protein, 9 aa, Chlamydia pneumoniae
  BINDING (1)..(9): CPN0681 immunogenic peptide
TLLHEFNQL

SEQ ID NO: 148 - protein, 9 aa, Chlamydia pneumoniae
  BINDING (1)..(9): CPN0681 immunogenic peptide
FGQSPFAPL

SEQ ID NO: 149 - protein, 9 aa, Chlamydia trachomatis
  BINDING (1)..(9): CT149 immunogenic peptide
FLGAAPAQM

SEQ ID NO: 150 - protein, 9 aa, Chlamydia trachomatis
  BINDING (1)..(9): CT149 immunogenic peptide
FLGIQDHIL

SEQ ID NO: 151 - protein, 9 aa, Chlamydia trachomatis
  BINDING (1)..(9): CT149 immunogenic peptide
LLTANGIAV

SEQ ID NO: 152 - protein, 9 aa, Chlamydia trachomatis
  BINDING (1)..(9): CT149 immunogenic peptide
SLPRRIPVL

SEQ ID NO: 153 - protein, 9 aa, Chlamydia trachomatis
  BINDING (1)..(9): CT149 immunogenic peptide
GLQEHCRGV

SEQ ID NO: 154 - protein, 9 aa, Chlamydia trachomatis
  BINDING (1)..(9): CT149 immunogenic peptide
SLGCHTTIH

SEQ ID NO: 155 - protein, 9 aa, Chlamydia trachomatis
  BINDING (1)..(9): CT149 immunogenic peptide
ILTHFQSNL

SEQ ID NO: 156 - protein, 9 aa, Chlamydia trachomatis
  BINDING (1)..(9): CT149 immunogenic peptide
VLSCGYNLV

SEQ ID NO: 157 - protein, 9 aa, Chlamydia trachomatis
  BINDING (1)..(9): CT149 immunogenic peptide
LLKEICATI

SEQ ID NO: 158 - protein, 9 aa, Chlamydia trachomatis
  BINDING (1)..(9): CT149 immunogenic peptide
RLFLGAAPA

SEQ ID NO: 159 - protein, 9 aa, Chlamydia trachomatis
  BINDING (1)..(9): CT149 immunogenic peptide
ATVAKYPEV

SEQ ID NO: 160 - protein, 9 aa, Chlamydia trachomatis
  BINDING (1)..(9): CT149 immunogenic peptide
LLSGSGFAA

SEQ ID NO: 161 - protein, 9 aa, Chlamydia trachomatis
  BINDING (1)..(9): CT149 immunogenic peptide
LTANGIAVA

SEQ ID NO: 162 - protein, 9 aa, Chlamydia trachomatis
  BINDING (1)..(9): CT149 immunogenic peptide
SGFAAPVEV

SEQ ID NO: 163 - protein, 9 aa, Chlamydia trachomatis
  BINDING (1)..(9): CT500 immunogenic peptide
FMISGPVVV

SEQ ID NO: 164 - protein, 9 aa, Chlamydia trachomatis
  BINDING (1)..(9): CT500 immunogenic peptide
ALFGESIGV

SEQ ID NO: 165 - protein, 9 aa, Chlamydia trachomatis
  BINDING (1)..(9): CT500 immunogenic peptide
SLENAAIEV

SEQ ID NO: 166 - protein, 9 aa, Chlamydia trachomatis
  BINDING (1)..(9): CT500 immunogenic peptide
LMGATNPKE

SEQ ID NO: 167 - protein, 9 aa, Chlamydia trachomatis
  BINDING (1)..(9): CT500 immunogenic peptide
RIAAMKMVH

SEQ ID NO: 168 - protein, 9 aa, Chlamydia trachomatis
  BINDING (1)..(9): CT671 immunogenic peptide
ALVETPMAV

SEQ ID NO: 169 - protein, 9 aa, Chlamydia trachomatis
  BINDING (1)..(9): CT671 immunogenic peptide
FCGANLTLV

SEQ ID NO: 170 - protein, 9 aa, Chlamydia trachomatis
  BINDING (1)..(9): CT671 immunogenic peptide
SLKARQLNL

SEQ ID NO: 171 - protein, 9 aa, Chlamydia trachomatis
  BINDING (1)..(9): CT671 immunogenic peptide
QLTEATQLV

SEQ ID NO: 172 - protein, 9 aa, Chlamydia trachomatis
  BINDING (1)..(9): CT671 immunogenic peptide
DLQWVEQLV

SEQ ID NO: 173 - protein, 9 aa, Chlamydia trachomatis
  BINDING (1)..(9): CT671 immunogenic peptide
IVLDNSNTV

SEQ ID NO: 174 - protein, 9 aa, Chlamydia trachomatis
  BINDING (1)..(9): CT841 immunogenic peptide
LLFGVIFGV

SEQ ID NO: 175 - protein, 9 aa, Chlamydia trachomatis
  BINDING (1)..(9): CT841 immunogenic peptide
LLAKGQNKV

SEQ ID NO: 176 - protein, 9 aa, Chlamydia trachomatis
  BINDING (1)..(9)
FTFMPIILV

SEQ ID NO: 177 - protein, 9 aa, Chlamydia trachomatis
  BINDING (1)..(9): CT841 immunogenic peptide
FLGDVSSGA

SEQ ID NO: 178 - protein, 9 aa, Chlamydia trachomatis
  BINDING (1)..(9): CT841 immunogenic peptide
LLDAAYQRA

SEQ ID NO: 179 - protein, 9 aa, Chlamydia trachomatis
  BINDING (1)..(9): CT841 immunogenic peptide
GMSDHLGTV

SEQ ID NO: 180 - protein, 9 aa, Chlamydia trachomatis
  BINDING (1)..(9): CT841 immunogenic peptide
SLGATHFLP

SEQ ID NO: 181 - protein, 9 aa, Chlamydia trachomatis
  BINDING (1)..(9): CT841 immunogenic peptide
NLAALENRV

SEQ ID NO: 182 - protein, 9 aa, Chlamydia trachomatis
  BINDING (1)..(9): CT841 immunogenic peptide
YLFTFMPII

SEQ ID NO: 183 - protein, 9 aa, Chlamydia trachomatis
  BINDING (1)..(9): CT841 immunogenic peptide
FPTAFFFLL

SEQ ID NO: 184 - protein, 9 aa, Chlamydia trachomatis
  BINDING (1)..(9): CT841 immunogenic peptide
ILMAATNRP

SEQ ID NO: 185 - protein, 9 aa, Chlamydia trachomatis
  BINDING (1)..(9): CT841 immunogenic peptide
KTALNDNLV

SEQ ID NO: 186 - protein, 9 aa, Chlamydia trachomatis
  BINDING (1)..(9): CT841 immunogenic peptide
LLNEAALLA

SEQ ID NO: 187 - protein, 9 aa, Chlamydia trachomatis
  BINDING (1)..(9): CT841 immunogenic peptide
ELYDQLAVL

SEQ ID NO: 188 - protein, 9 aa, Chlamydia trachomatis
  BINDING (1)..(9): CT841 immunogenic peptide
ALEKQDPEV

SEQ ID NO: 189 - protein, 9 aa, Chlamydia trachomatis
  BINDING (1)..(9): CT841 immunogenic peptide
SLGGRIPKG

SEQ ID NO: 190 - protein, 9 aa, Chlamydia trachomatis
  BINDING (1)..(9): CT841 immunogenic peptide
FMPIILVLL

SEQ ID NO: 191 - protein, 9 aa, Chlamydia trachomatis
  BINDING (1)..(9): CT841 immunogenic peptide
LLAARKDRT

SEQ ID NO: 192 - protein, 9 aa, Chlamydia trachomatis
  BINDING (1)..(9): CT841 immunogenic peptide
VTFADVAGI

SEQ ID NO: 193 - protein, 9 aa, Chlamydia trachomatis
  BINDING (1)..(9): CT841 immunogenic peptide
YTISPRTDV

SEQ ID NO: 194 - protein, 9 aa, Chlamydia trachomatis
  BINDING (1)..(9): CT841 immunogenic peptide
LIGAPGTGK

(19) United States
(12) Patent Application Publication
(10) Pub. No.: US 2003/0199438 A1
(43) Pub. Date: Oct. 23, 2003

(54) Method for identification of proteins from intracellular bacteria

(76) Inventors: Allan Christian Shaw, Aarhus C (DK); Brian Berg Vandahl, Aarhus N (DK)

Correspondence Address: DARBY & DARBY P.C., P. O. BOX 5257, NEW YORK, NY, 10150-5257, US

(21) Appl. No.: 10/119,536

(22) Filed: Apr. 9, 2002

Related U.S. Application Data
(60) Provisional application No. 60/282,513, filed on Apr. 9, 2001.

(30) Foreign Application Priority Data
Apr. 9, 2001 (DK) PA 2001 00581

(51) Int. Cl. A61K 38/16; C12Q 1/04

(52) U.S. Cl. 514/12; 435/34

(57) ABSTRACT

The present invention relates to a novel combination of methods that enables identification of proteins secreted from intracellular bacteria regardless of the secretion pathway. The invention further provides proteins that are identified by these methods. Secreted proteins are known to be suitable candidates for inclusion in immunogenic compositions and/or diagnostic purposes. The invention also provides peptide epitopes (T-cell epitopes) from the identified secreted proteins, as well as nucleic acid compounds that encode the proteins. The invention further comprises various applications of the proteins or fragments thereof, such as pharmaceutical and diagnostic applications.

### TECHNICAL FIELD OF THE INVENTION

The present invention relates to a novel combination of methods that enables identification of proteins secreted from intracellular bacteria regardless of the secretion pathway. The invention further provides proteins that are identified by these methods. Secreted proteins are known to be suitable candidates for inclusion in immunogenic compositions and/or diagnostic purposes. The invention also provides peptide epitopes (T-cell epitopes) from the identified secreted proteins, as well as nucleic acid compounds that encode the proteins. The invention further comprises various applications of the proteins or fragments thereof, such as pharmaceutical and diagnostic applications.

### BACKGROUND OF THE INVENTION

The Chlamydia are obligate intracellular bacteria, which multiply inside eukaryotic host cells and are important human pathogens. The order Chlamydiales comprises one family (Chlamydiaceae) containing one genus (Chlamydia), which is divided into the four species: C. trachomatis, C. pneumoniae, C. psittaci and C. pecorum.

The human pathogenic serovars of C. trachomatis are divided into: A-C which afflict ocular diseases; and D to K, which are sexually transmitted and causes urethritis or complications such as salpingitis, epidymitis and ectopic pregnancies; and L1 to L3 which cause a severe systemic infection, lymphogranuloma venereum (LGV). The human pathogen C. pneumoniae is responsible for respiratory tract infections causing bronchitis and pneumonia and has recently been associated with the development of atherosclerosis (Saikku et al, 1988 [1.], Shor et al., 1992 [2.]).

If left untreated Chlamydia infections may become chronic with severe complications such as sterility, blindness and potentially thrombosis.

Due to the intracellular developmental cycle persistent Chlamydia infections may cause an aberrant immune response, which fails to clear the organisms. Many immunogenic Chlamydia proteins have been considered vaccine candidates, especially surface exposed proteins such as the major outer membrane protein (MOMP) that is immunodominant in C. trachomatis [7.], but also stress response proteins such as Hsp60 [8.]. However, none of these candidates have been proven efficient in vaccine trials.

A likely explanation for the limited humoral response and little protective immunity is the intracellular nature of the organism. An alternative approach is therefore to find proteins, which are recognizable by the cell-mediated immune system, which has been shown to be pivotal in the resolution of chlamydial infection primarily through the effect of cytotoxic T-lymphocytes (CTL) (Iguitseme et al 1994 [9.])

Great attention has been drawn to secreted proteins since these may be processed in the host cell proteasomes and presented as MHC-class I antigens on the surface of cells and thus represent obvious vaccine targets (Hess and Kaufmann, 1993 [45]). An example of this was shown for Yersinia infected cells, which presents an epitope of the YopH effector to MHC restricted cytotoxic T-lymphocytes (CTL). (Starnbach & Bevan, 1995 [14.]) The interaction between Chlamydia and the host cell is essential for the intracellular survival and propagation of the bacteria.

Complete and searchable Chlamydia genomes exist for C. trachomatis serovar D (Stephens et al, 1998 [4.]) (comprising 894 predicted open reading frames (ORFs)) and C. pneumoniae VR1310 (comprising 1073 ORFs) (Kalman et al 1999 [5.]). In addition complete genomes of C. trachomatis MoPn (Read et al., 2000 [12.]) C. pneumoniae AR39 (Read et al [12.] and C. pneumoniae J138 (Shirai et al. 2000 [13.] are public available. From the genome sequence it is known that Chlamydia posses genes involved in secretion mechanisms including several genes with homology to type III secretion genes from other organisms (Stephens et al., 1998 [4.] and Kalman et al., 1999 [5.]).

Candidates for secreted effector proteins are likely to be present in Type III secretion subclusters (Subtil et al., 2000) [10.]. This view was recently illustrated by the discovery of the Type III secretion characteristics of CopN (Fields & Hackstadt 2000) [11.] Type III secreted proteins, however, lack recognisable signal peptidase cleavage sites and no consensus sequence for proteins secreted by this system in Chlamydia has been recognized, such may be restricted to the particular organism in question. Moreover, secreted proteins may be a functionally diverse group of proteins located in unpredictable locations in the genome (Subtil, 2000) [10.].

The present state of knowledge concerning secreted Chlamydia effector proteins is limited to proteins present in the inclusion membrane including members of the Inc family (Rockey et al. 1995) [15.] [16.], CopN (Fields &Hackstadt, 2000 [11.]) and CT529 (Fling et al, 2001) [37.].

It has been shown that CD8+ T-cells specific for Chlamydia arise during an infection, meaning that Chlamydia proteins are exposed to the host cell cytoplasm which is a prerequisite for presentation of MHC class I antigens. CT529 has been identified from a genomic library by expression in a eukaryotic cell and recognition by a Chlamydia specific T-cell line (Probst) [41]. CT529 has been shown to contain an epitope, which in mouse vaccine experiments provides some protection against infection.

Expression in eukaryotic cells of a genomic Chlamydia trachomatis serovar L2 library by transfection with a viral vector and subsequently screening with Chlamydia specific T-cells for the detection of proteins comprising MHC class I restricted epitopes has been described in The International Patent Application No. WO 00/34483 (Probst) [41]. This method has resulted in the identification of five positive clones, CT529 was contained in one of these, another clone contained three open reading frames but the remaining three clones have not been described further. Drawbacks of such a screening is the eukaryotic expression of bacterial proteins which may differ from bacterial expression in a way that alters the probability of processing in the proteasome and the very presentation as MHC antigens, and the maintenance and stimulation of T-cell clones differ from the in vivo situation and clones recognizing proteins, which are not accessible during a normal infection may result in false positives. Other approaches in the above patent application concerns identification of candidates for a vaccine directed against the humoral immune defence.

When searching for proteins secreted from intracellular bacteria, the straightforward idea would be to isolate the cytoplasm from infected host cells and look for bacterial proteins. However, this strategy cannot be employed for Chlamydia due to the fragility of the chlamydial reticulate body. Another approach would be to identify pathogenicity factors, which are often secreted proteins, by transposon analysis. However, it is not possible to transfect Chlamydia. No strategy exists that can predict which proteins are secreted and genes encoding effector proteins suitable for vaccine development may be located in unpredictable locations in the genome.

Thus, there is a need for a reliable system, which can limit the number of vaccine candidates in a cost efficient way and which involves a minimum of experimental steps.

In [18] the effect of IFN-γ C. trachomatis A and L2 protein expression was investigated by means of [35S]-methionine/cysteine labelling of C. trachomatis proteins in combination with autoradiography following 2D-gel electrophoresis. IFN-added during the infection of HeLa cell cultures with C. trahomatis A resulted in a pronounced down-regulation of several C. trachomatis A proteins, wheras this effect was not apparent for C. trachomatis L2. IFN-γ-dependent induction of ˜30 and ˜40 kDa proteins in both C. trachomatis A and L2 was observed. The induction of these proteins was antagonized by addition of super-fysiological amounts of L-tryptophan to the growth medium. This indicated that the IFN-γ mediated inducibility of these C. trachomatis proteins is associated with IFN-γ mediated up-regulation of the tryptophan degrading host cell enzyme indoleamine 2, 3 dioxygenase. One of the IFN-induced C. trachomatis proteins migrated with a significantly lower molecular weight in C. trachomatis A compared to C. trachomatis L2.

In [19] The IFN-γ inducible C. trachomatis A and L2 proteins described previously (Shaw et al. 1999) were further characterized. Using MALDI-TOF mass spectrometry followed by database search the proteins were identified as the C. trachomatis tryptophan synthase alpha (TrpA) and beta (TrpB) subunits from preparative 2D-gels. The proteins were also induced by IFN- in C. trachomatis D and the induction was prevented by addition of super-fysiological amounts of L-tryptophan in all three serovars. TrpA in C. trachomatis A migrated with a lower molecular weight in C. trachomatis A compared to C. trachomatis D and L2. C. trachomatis A and C TrpA are truncated by ˜7.7 kDa compared to C. trachomatis D and L2 TrpA as revealed by analysis of the trpA gene from these C. trachomatis serovars. The truncation or absence of tryptophan synthase in the trachoma causing serovars (C. trachomatis A, B and C) may impair the trytophan synthesizing ability and render these serovars more susceptible to IFN-γ mediated tryptophan depletion. This can explain differences seen in pathogenesis among human C. trachomatis serovars.

In [43] the host cell proteasomal degradation of a previously described secreted protein (p60) of the intracellular bacterium Listeria monocytogenes was investigated. The general strategy used was based on pulse chase assays using [35 S]-methionine/cysteine labelling in the presence or absence of two peptidealdehydes: N-acetyl-Leu-Leu-norleucinal(LLnL) and (benzyloxycarbonyl)-Leu-Leu-phenylalaninal (Z-LLF), which inhibit the proteolytic activity of the eukaryotic proteasome. Polyclonal antibody raised against p60 wsa used to immunoprecipitate p60 from proteosome inhibitor treated and non-treated lysates of L. monocytogenes-infected J774 cells. Evaluation of autoradiographs of immunoprecipitated labelled p60 separated by one-dimensional SDS PAGE suggested that proteasome inhibitors were able to inhibit proteasome degradation of p60. The number of p60-CTL epitopes per infected cell decreased upon treatment with LLnL and Z-LLF. This suggested a link between inhibition of proteasomal degradation of p60 and p60-CTL epitope production.

In [44] mechanisms behind protective immunity and general features of the cellular immunereponse towards intracellular microorganisms were described with the focus on development of viable recombinant vaccines against intracellular microbes. Strategies for developing antigen delivery systems were discussed with emphasis on Mycobacterium bovis BCG and Salmonella typhi aroA⁻. These non-virulent intracellular bacteria can be genetically modified to deliver antigens, which may serve as targets for a vaccine by immune recognization. The authors point out the advantages of using secreted proteins as targets for the development of a vaccine as these proteins will be processed and presented to the cell-mediated immunesystem while the bacterium still replicates inside the host cell.

### SUMMARY OF THE INVENTION

The present invention comprises the identification of secreted proteins by a novel combination of methods. The described combination of methods constitutes a secretome (the collection of secreted proteins) by subtraction of the proteome of intracellular bacterial proteins from the total proteome of bacterial proteins present in infected cells.

The bacterial proteins are selectively visualized by pulse labelling in the presence of an inhibitor of eukaryotic protein synthesis followed by two dimensional electrophoresis and autoradiography. Protein profiles of purified bacteria are compared to protein profiles of the total lysate of infected cells and the protein spots present in the differential image, the secretome, are identified from gels loaded with total lysate of infected cells by advanced mass spectrometric methods.

The identified secreted proteins are further analysed by advanced artificial neural networks to provide peptide sequences that are predicted to be good T-cell epitopes.

Furthermore, proteins are selected for which the turnover is delayed by inhibitors of the host cell proteasome since these proteins are especially likely to be degraded in the host cell proteasome and presented as MHC class I antigens on the host cell surface.

Compared to other strategies for identification of vaccine candidate proteins and epitopes, the present invention provides a limitation of the number of candidates, which can only be obtained by the novel combination of methods.

The invention is based upon the following observations:

Proteins that are secreted from an intracellular bacteria into the host cell will be absent from purified bacteria but present in whole lysates of infected cells.

2D protein profiles of purified bacteria as well as whole lysates of infected cells can be made visible by two-dimensional electrophoresis using specific pulse-labelling of bacterial proteins.

Subtraction of the 2D protein profile of purified bacteria from the 2D protein profile of bacterial proteins present in the host cell enables the identification of secreted proteins by mass spectrometri methods.

Proteins that are secreted from an intracellular bacteria, which are processed by the host cell proteasome are likely to generate MHC class I antigens, which are capable of activating T-cells.

Secreted proteins, which exhibit a prolonged turnover in response to the addition of inhibitors of the eukaryotic proteasome, are likely to be presented at the host cell surface. The identification of such proteins is enabled by analysis of 2D proteins profiles.

T-cell epitopes can be predicted by artificial neural networks trained to recognize peptides that have a high affinity for the MHC class I complex.

The following definitions are used in connection with the present invention:

Definitions

Secretome

Proteins that are very likely to be secreted from an intracellular bacteria.

(Type III) Secretion Subclusters

A cluster of Chlamydia genes which contains genes that have significant homology to type III secretion gene from other organisms. By the definition of secretion cluster is meant a collection of ORFs (open reading frames) with known or unknown function, which are located up to four genes away from any gene with known homology to genes involved in Type III secretion in other bacteria (e.g. Salmonella, Shigella, Yersinia.)

Proteasome Inhibitor

Any chemical synthesized or naturally occurring compound, which is able to reversibly or irreversibly inhibit the proteolytic activity of the activated 26S eukaryotic proteasome. Persons skilled in the art will recognize that proteolytic activity of the proteasome contains several different activities (e.g. chymotrypsin-like activity, which cleaves after large hydrophobic residues, trypsin-like activity, which cleaves after basic residues, post-glutamy′l hydrolase activity, which cleaves after acidic residue, BrAAP, which cleaves preferentially after branched-chain amino acid, SNAAP, which cleaves after small neutral amino acids of subunits). Several compounds known to inhibit the proteasome are commercially available and mostly include cell permeable peptide based inhibitors (e.g. peptide aldehydes, peptide vinol sulphones). Peptide based inhibitors act as transition state analogs, which form an adduct with the proteasome's active sites, whereas the naturally occuring clasto-Lactacystin-β-lactone exerts a proteasome inhibiting effect by means of irreversible modification of the active sites of the proteasome subunits. These compounds and combinations hereof can potentially be used to successfully inhibit proteasome function and MHC-class I antigen processing (e.g. MG115, MG132, MG262, PSI, clasto-Lactacystin-β-lactone, Epoxymycin). The application of proteasome inhibitors may be used at any time point throughout the developmental cycle of Chlamydia either before, during or after pulse labelling or chase is performed.

Host Cells

A host cell is any eukaryotic cell, which can be infected with an intracellular bacteria. A person skilled in the art will recognize that a wide range of immortalized cell lines will be suitable hosts for infection with Chlamydia including epithelial cell lines (e.g. HeLa, Hep-2, BHK cells) or immortalized mononuclear cell lines, e.g. U-937. Immortalized host cell may be obtained from naturally occurring carcinoma or by transformation of primary cells with virus, which carries oncogenic genes, which results in unlimited cell division and growth (eg. SV40). The definition of host cells also includes primary epithelial or endothelial mammalian cell lines, which can be obtained from living mammals or by autopsy, and propagated for a limited time in vitro, and organ cell culture.

Genetically Modified Host Cell

A person skilled in the art will acknowledge that appropriate host cell also includes host cell, which have been genetically modified to overexpress or suppress genes, which are relevant in context of chlamydial vaccine development, e.g. genes encoding proteasome subunits or other genes encoding functionally important proteins involved in MHC-class I presentation.

Proteasome

The proteasome is the central enzyme complex of non-lysosomal protein degradation being an essential component of the ATP-dependent proteolytic pathway catalyzing the rapid degradation of many rate-limiting enzymes, transcriptional regulators and critical regulatory proteins. In eukaryots it is essential for the rapid elimination of abnormal proteins, aggregated, unfolded or normal host cell proteins as well as proteins coming from intracellular bacteria located in the host cell. The proteasome in higher eukaryotes is critically involved in MHC class I antigen processing by degrading proteins to peptides which are delivered to the host cell surface for presentation as T-cell epitopes.

Pulse-Labelling

By labelling of proteins is meant incorporation of amino acids containing radioactive isotopes (e.g. L-[³⁵S]-methionine, L-[methyl-3H]-methionine, L-[methyl-¹⁴C]-methionine, [³⁵S]-cysteine, [³H]-tryptophane or combinations hereof) in bacterial proteins in a period of time (eg. 0.5 hours, 1 hours, 2 hours, 4 hours, 6 hours) during which the host cell protein synthesis is inhibited using a sufficient concentration of inhibitors of eukaryotic protein synthesis. The labelling can be performed throughout the Chlamydia developmental cycle. The labelling medium must be sufficiently enriched with nutrients to allow growth of both the host cell and the pathogen during the time in which the infected cells grow. The inhibition of host cell protein synthesis may be accomplished through addition of cyclohexamide or other inhibitors of host cell protein synthesis (e.g. emetine) during the labelling period, with the effect of allowing incorporation of the radioactive amino acid only in the protein synthesizing intracellular bacterium. Protein degradation can be prolonged by adding cell-permeable inhibitors of protein degradation to the growth medium during the labelling period. It is to be noted that the present invention is not limited to the use of radioactive labelling.

Pulse-Chase

By chasing labelled proteins after there synthesis the turnover time can be estimated, e.g. the time span after which the amount of protein synthesised during the labelling period is degraded by preferably more than 75% (e.g. 80%, 90%, 95%, 99%, 100%). This estimation is performed by measuring the optical density of a given protein spot in the gel before and after a chase period and reveals how long time the protein is present in the infected cell. The chase is performed by replacing the labelling medium with a growth medium without the radioactive amino acid and harvesting the infected cells to different time points after labelling. A chase can be performed for varying periods after the labelling (e.g. 0.5 hours, 1 hours, 1.5 hours, 2 hours, 6 hours, 12 hours, 24 hours, 72 hours) and after labelling at various time points in order to determine how long time the protein is present in the infected cell. The mature form of certain proteins may be processed from a propeptide, and thus accumulate during the chase period instead of decreasing in amount. Under these circumstances the mature protein may accumulate before the degradation can be visualized during the chase periods. The time span, during which the protein is degraded, can be prolonged by adding cell-permeable inhibitors of protein degradation to the growth medium during the chase.

Lysis Buffer

A lysis buffer for use in the present invention is a buffer used to lyse infected cells and solubilize proteins prior to two-dimensional gel electrophoresis.

The lysis buffer contains 9 M Urea, 4% w/v 3-[(3-cholamidopropyl)dimethylammonium]-1-propanesulfonate (CHAPS; Roche, Germany), 40 mM Tris Base, 65 mM DTE and 2% vol/vol Pharmalyte 3-10 (Amersham Pharmacia Biotech). For the enrichment of high molecular weight and hydrophobic proteins the lysis buffer alternatively contains 7 M urea, 2 M thiourea, 4% w/v 3-[(3-cholamidopropyl)dimethylammonium]-1-pro-panesulfonate (CHAPS; Boehringer Mannheim, Germany), 40 mM Tris Base, 65 mM dithioerythretiol (DTE) and 2% vol/vol Pharmalyte 3-10 (Amersham Pharmacia Biotech).

It is recognized that it is possible to alter the lysis buffer in order to increase the solubility of certain proteins (e.g. thiourea will increase the solubility of hydrophobic and high molecular weight proteins).

Secreted Effector Protein

By the term secreted effector protein is meant any protein, which is secreted by the bacteria into the host cell cytoplasm or any intracellular organelle. Secreted effector protein may have great influence on the host/pathogen relation and due to its presence in the host cell cytoplasm may be targeted to the proteasome and presented as MHC-class I antigens on the surface of the host cell. Secreted effector proteins may be secreted by one of several Sec-dependent or independent systems (e.g. Type I, Type II, Type III, Type IV) described in the literature.

Intracellular Bacteria

Any bacteria, which has the ability to infect and propagate inside a eukaryotic host cell (e.g. Chlamydia, Salmonella, Shigella, Listeria, Legionella, Yersinia). The definition includes intracellular bacteria, which are obligate intracellular meaning that they may only live and propagate using an eukaryotic host cell or facultative intracellular meaning that they may both survive in an extracellular as well as an intracellular milieu.

Elementary Body (EB)

The collection of Chlamydia bacteria purified by ultracentrifugation and characterized by electron microscopy as being about 300 nm in diameter and having a condensed nucleus.

Reticulate Body (RB)

The collection of Chlamydia bacteria purified by ultracentrifugation and characterised by electron microscopy as being about 1000 nm in diameter and having a normal bacterial nucleus.

Analytical Gel

Any 2D-PAGE gel, which is loaded with a protein sample amount necessary to visualize proteins. The amount applied for analytical purposes in the herein described examples is typically 200.000 to 300.000 counts per minutes (cpm) or >100 μg protein for stained gels (e.g. silver stained, Coomasie stained).

Significantly Decreased Intensity/Amount

A reproducible detectable reduction preferably greater than 10% (e.g. 20%, 35%, 50%, 65%, 80%, 90%, 100%) in the optical density integrated over total area of a given spot localized on a 2D-PAGE gel.

Significantly Increased Intensity/Amount

A reproducible detectable increase preferably greater than 10% (e.g. 20%, 30%, 45%, 60%, 75%, 90%, 100%, 150%, 200%,300% or more) in the optical density integrated over total area of a given spot localized on a 2D-PAGE gel.

Preparative Gel

A 2D-PAGE gel, which is loaded with a protein sample amount necessary to allow identification of specific protein spots by one of the herein described identification methods (e.g. MALDI-MS, ESI-Q-TOF, Edman degradation). Typically >500 μg is applied on gels for preparative purposes depending on the immobilised pH gradient used. The definition of preparative gels used in the present invention includes gels with proteins that are unfixed, fixed using staining protocols (e.g. silver staining, Coomasie staining) or electroblotted on to PVDF membranes. It is possible to visualize proteins on preparative gels by applying a background of labelled proteins to the preparative gels, which are separated along with the non-labelled proteins. It is also possible to compare such gels with analytical gels in order to excise the exact protein of interest.

Vaccine Candidate

A protein, which based on results obtained by the methods of the present invention is potentially secreted from an intracellular bacteria. Secreted proteins are accessible for degradation by the host cell proteasome and peptides derived from these proteins may therefore be presented as MHC-class I antigens at the surface of the infected cell, thus being recognizable by T-cells. Such proteins will therefore be obvious targets for the development of a vaccine against the intracellular bacteria. A protein described as a vaccine candidate may also serve useful as a component of a diagnostic test.

Vaccine

In the present invention the term vaccine is to be understood in its broadest sense as an immunogenic composition, which is able to elicit an adaptive immune response (humoral or cellular). Vaccines candidates, which are able to elicit an adaptive immune response may be administered to animal or human recipient as injectables either in the form of solutions, suspensions or as emulsions. The vaccine candidate being the active component of the immunogenic composition may be mixed with pharmaceutical acceptable excipients such as water, saline, glycerol and ethanol before injection into the recipient. Injection may be carried out in different ways (e.g. subcutaneously or intramuscularly). Vaccine candidates may serve as a vaccine either i) in its full length, or ii) as a source for providing immunogenic fragments, e.g. T-cell epitopes. It is acknowledged that specific proteins or peptides alone or in combination with other proteins or peptides may be administered to an animal or human recipient and serve as a vaccine.

Furthermore a DNA fragment encoding a vaccine candidate protein can be cloned in a vector, which can be introduced by injection into an animal or a human recipient. The DNA fragment is taken up by e.g. muscle cells and expressed under the control of a promoter, which will be active in eukaryots. In this so-called DNA vaccine the expressed DNA fragment is capable of stimulating the immune system.

MHC Class I Antigen

A major histocompatibility class I antigen comprises a peptide diverged from a protein which is exposed to the host cell cytoplasm, which is conjugated to a heterodimeric MHC class I molecule in the endoplasmatic reticulum and is presented on the surface of the cell bound in the grove of HLA complex (in humans) where it may serve as a T-cell epitope. The majority of MHC-class I-presented peptides are diverged from a protein processed in the cytoplasm of the host cell by the activated 26S-proteasome.

HLA

Human leukocyte antigen, the name for the human major histocompatibility complex.

T-Cell Epitope

A peptide of short length, which bound to a dimeric MHC class I molecule on the surface of a cell, can be recognized by the receptor of a specific cytotoxic T-cell, e.g. consisting of typically 8-10 amino acids.

Whole Cell Lysates

Infected host cells harvested directly in lysis buffer, without prior purification or fractionation. It is recognized that whole cell lysates may be obtained at any time point throughout the chiamydial developmental cycle and that the whole cell lysates will contain a mixture of all proteins present in the infected host cell including those coming from the bacteria.

Purified Bacteria

Bacteria, which is purified from infected cells. Using Chlamydia as an example, it is possible to purify both RB and EB as well as intermediate forms of Chlamydia by density gradient ultra centrifugation methods, depending on the time point in the developmental cycle at which the Chlamydia are harvested. The purity can be determined by electron microscopy. In the present invention the purity is also readily verified on silverstained 2D gels by estimating the contribution contaminating highly abundant host cell proteins (e.g. actin, beta-tubulin, alfa-tubulin, calreticulin) to the total optical density of all proteins present on the gels.

Identified Protein

Names on proteins identified using identification methods based on mass spectrometry or Edman degradation are indicated following a nomenclature according to and compatible with the one used for genes in the Chlamydia Genome Project available http://socrates.berkeley.edu: 4231/and as published in

i) Stephens, R. S., Kalman, S., Lammel, C., Fan, J., Marathe, R., Aravind, L., Mitchell, W., Olinger, L., Tatusov, R. L., Zhao, Q., Koonin, E. V., Davis, R. W. (1998) Genome sequence of an obligate intracellular pathogen of humans: Chlamydia trachomatis. Science 282: 754-759

ii) Kalman, S., Mitchell, W., Marathe, R., Lammel, C., Fan, J., Hyman, R. W., Olinger, L., Grimwood, J., Davis, R. W., Stephens, R. S (1999). Comparative genomes of Chlamydia pneumoniae and C. trachomatis. Nat Gent. 21: 385-389

ELISPOT

In the ELISPOT method, T-cells that have been stimulated with antigen in vitro are incubated in microtitter wells pre-coated with anti-cytokine (e.g. IFN-g, IL-6, TNF-a) antibody. After a period of incubation the local production of cytokines around activated T-cells can be visualized by adding a secondary antibody conjugated to an enzyme such as horseradish peroxidase alkaline peroxidase. An estimation of the production of cytokines is done by finally adding a substrate that will be enzymatically converted into a coloured product thus allowing cytokine producing cells to be visualized.

Adjuvant

By adjuvant is meant an emulsion, which contains a specific immunogen, which can elicit an immuneresponse in a mammal recipient (e.g. Freunds adjuvant). It is recognized that an adjuvant together with the immunogen can be supplemented with components such as dried bacteria or bacterial products, which will enhance the immune response in an immunized mammal. Alternatively, immunomodulating substances such as lymphokines (e.g., IFNg, IL12) or poly I:C may also be administered together with the immunogen and adjuvant.

Seroconversion

The development of different classes or subclasses of antibodies in response to an antigen.

Micro Immuno Fluorescence (MIF or Micro-IF)

An assay, which measures antibodies against fixed bacteria or proteins by immuno fluorescence microscopy.

Immunogenic

A protein or peptide is immunogenic if it can elicit an adaptive immune response upon injection into a person or an animal.

### DESCRIPTION OF THE DRAWINGS

FIG. 1A shows an example of a gel image of C. trachomatis D proteins from whole cell lysates [35S]-labelled from 22-24 hours post infection (h.p.i.), harvested immediately after labelling and separated by 2D-PAGE. Black arrows mark proteins which intensities are significantly reduced on gels with proteins from purified EB (elementary bodies) labelled from 22-24 h.p.i. and purified 72 h.p.i. The pI and Mw characteristics of the marked spots (DT1-77) are listed in Table I.

FIGS. 1B-E show examples of identified vaccine candidates and their presence in C. trachomatis D at different times after synthesis shown by enlargements of selected areas from FIG. 1A. The protein turnover time was estimated by chasing at different times after labelling throughout the developmental cycle until purification of EB. The upper scale indicates points in time after labelling starting with zero; the lower scale represents the time in hours post infection starting with 24 hours. FIG. 1B: DT1 and DT2 (CT668). FIG. 1C: DT8. FIG. 1D: DT7 (upper arrow) and DT11 (lower arrow) (both identified as CT610). FIG. 1E: DT3 (lower arrow, CT783),DT4 (upper arrow, CT858).

FIG. 2A shows an example of gel image of C. pneumoniae VR1310 proteins from whole cell lysates [35S]-labelled from 55-57 h.p.i., harvested immediately after labelling and separated by 2D-PAGE. Black arrows mark proteins which intensities are significantly reduced on gels of proteins from EB labelled at two hour periods throughout the developmental cycle i.e. 6, 12, 24, 36, 42, 48, 54, 60 h.p.i. and purified 72 h.p.i. Arrows mark proteins which are significantly reduced in intensity on gels from purified EB. The pI and Mw characteristics of the marked spots (CP1-91) are listed in Table II.

FIG. 2B shows an enlarged section of FIG. 2A.

FIG. 2C shows corresponding enlargement of image of 2D-PAGE separated EB proteins labelled as described above. The section enlarged in FIGS. 2B and 2C gives an example of two proteins CP63 (identified as CPN1016) and CP65, which are present in whole cell lysates but not in EB. The encircled spot is present in both EB and whole cell lysates and has been identified as polypeptide deformylase.

FIG. 3A shows a peptide mass fingerprint used to identify spot no. DT1 as the hypothetical protein CT668. Hollow arrows indicate peptides arising from autodigestion of trypsin. These peaks were used to make an internal calibration of the spectra. Black arrows indicate peptide masses matching the CT668 sequence. Doublet arrows indicate peptides originating from a human contaminating protein.

FIG. 3B shows results from using the identification software MS-Fit on the peptide masses obtained from FIG. 3A, showing that the highest-ranking C. trachomatis protein is CT668.

FIG. 4A shows a peptide mass spectrum generated by ESI-Q-TOF MS of spot DT1 comprising the double charged 1744.9 Da parent ion (R)KIVDWVSSGEEILNR(A) (black arrow), which matches the CT668 amino acid sequence. Dotted lines point at the Y-peptide ions that were generated by fragmentation of the parent ion. The deduced amino acid sequence is shown in bold one-letter code.

FIG. 4B shows a peptide mass spectrum generated by PSD MALDI MS of spot CP63. The 1919.8 Da parent ion (K)ELLFGWDLSQQTQQAR(L) was fragmented and gave rise to several peptides revealing the sequence. Two of these are exemplified by the 243.35 Da b₂-ion (EL) and 356.34 Da b₃-ion (ELL) that differ in mass by the mass of leucine (113 Da).

FIG. 5 shows the nucleotide sequence of the novel C. trachomatis specific vaccine protein candidate DT8 and corresponding amino acid sequence shown by one-letter code. Bold amino acid sequence was covered by sequence tags obtained by ESI-Q-TOF MS.

FIG. 6 shows pulse chase studies in combination with one the proteasome inhibitor MG-132. FIG. 6.1: Total gel image of a 2D-gel loaded with C. trachomatis D proteins labelled from 22-24 h.p.i. and chased for additionally 4 hours in the presence of 20 μM MG132. FIGS. 6A, 6B and 6C: Enlargements of regions containing C. trachomatis D proteins, which has a prolonged turnover time due to treatment with MG-132, when comparing chase studies performed with (chased+MG-132) or without (chased) MG-132. The first row represents protein profiles of infected cell harvested immediately after the two-hour labelling period. Note that the intensity of DT9, DT10 and DT11 is significantly greater on gels with whole cell lysates which are labelled and chased in the presence of MG132 compared to gels with whole cell lysates harvested immediately after labelling without MG132.

FIG. 7A shows a total gel image of IMB of whole cell lysates using PAb 245. A1: IMB showing reaction with spot no. DT4 and DT48, which are the C-terminal and N-terminal fragments of CT858, respectively. A2: Corresponding radio labelled background of the IMB.

FIG. 7B: IMB with PAb241 against YscN (B1) and PAb238 against CT668 (spot DT1 and DT2) (4). Corresponding autoradiography of IMB, showing co-localization with B2: YscN and B5: CT668. Localization of YscN(B3) and CT668(B6) on analytical 2D-gels.

FIG. 7C shows IMB with PAb255 against CT610 C1: Enlargement of 2D-gel blot showing that PAb 255 stains to rows of spots, the upper representing DT7 and the lower representing DT9, 10, 11 and 12. C2: Enlargement showing the effect of treatment of infected cells with MG132 for 6 hours prior to harvesting of the cells at 30 h.p.i. Note that MG132 treatment results in an clear relative increase in the abundance of DT9, DT10 and DT111 compared to the row representing DT7. C3: Corresponding labelled analytical gel. C4: SDS-PAGE IMB with PAb255: Lane a and c: 20 μg and 10 μg (respectively) of whole infected cell lysates harvested at 30 h.p.i. Lane b and d: 10 μg and 5 μg (respectively) of whole infected cell lysates treated for 6 hours with 50 μM MG132, prior to harvesting of the cells at 30 h.p.i.

FIG. 8 shows an indirect immunofluorescence microscopy showing sub-cellular localization of vaccine candidates. A and B: HeLa 229 cells infected with C. trachomatis D and fixed with formalin 24 h.p.i. C: HEp-2 cells infected with C. pneumoniae and fixed with formalin 72 h.p.i. Row I shows Normasky images. Row 2 shows reaction with MAb 32.3 against C. trachomatis MOMP visualized by rhodamine conjugated GAM IgG antibody. Row 3 shows reaction with rabbit polyclonal antibody specific for the vaccine candidate in question visualized by FITCH-conjugated GAR IgG antibody. The investigated vaccine candidates are A: DT8, B: CT858, C: CPN1016 (CT858 homologue in C. pneumoniae). White single headed arrows points at borders of Chlamydia inclusions in infected cells. Hollow arrows point at uninfected cells. Note that the CPN1016 shows the same sub-cellular localization and secreted characteristics as CT858.

FIG. 9 shows a genomic localization of examples of identified vaccine candidates from C. trachomatis D including identified proteins, which are located in type III secretion subclusters 1, 2 and 3.

FIG. 10 shows the position of C. trachomatis D secretion candidates in 2D-gel images from whole lysates of infected cells labelled from 22-24 h.p.i., RB labelled and purified at the same point in time and EB purified at 72 h.p.i. All gels have been made using non-linear pH 3-10 Immobiline Drystrips.

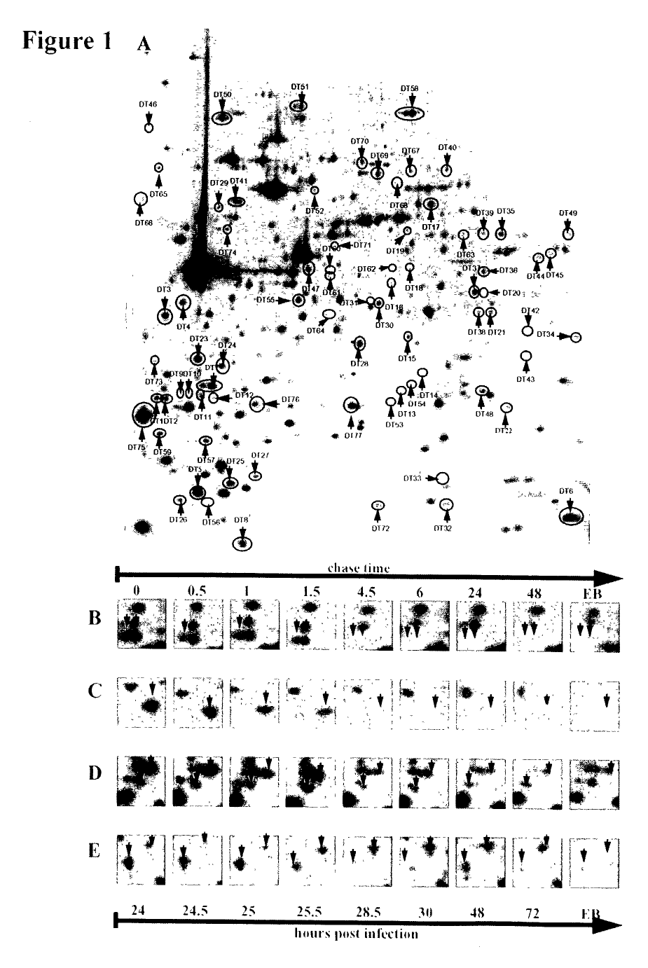

22-24 hpi: enlargements of the gel image shown in FIG. 1A of C. trachomatis D proteins from whole lysates of infected cells [35S]-labelled from 22-24 h.p.i. harvested immediately after labelling and separated by 2D-PAGE.

Purified RB: Corresponding regions from a gel image of C. trachomatis D proteins from bacteria purified as RB at 24 h.p.i immediately after labelling from 22-24 h.p.i.

Purified EB: Corresponding regions from a gel image of proteins from bacteria labelled from 22-24 h.p.i. and purified as EB 72 h.p.i.

In rows A-G the secretion candidates DT4, DT48, DT23, DT76, DT77, DT47 and DT75 have been encircled.

FIG. 11 shows the position of C. pneumoniae VR1310 secretion candidates in 2D-gel images from whole lysates of infected cells labelled from 55-57 h.p.i., purified EB, whole lysates of infected cells labelled at 34-36 h.p.i. and RB labelled and purified at the same point in time:

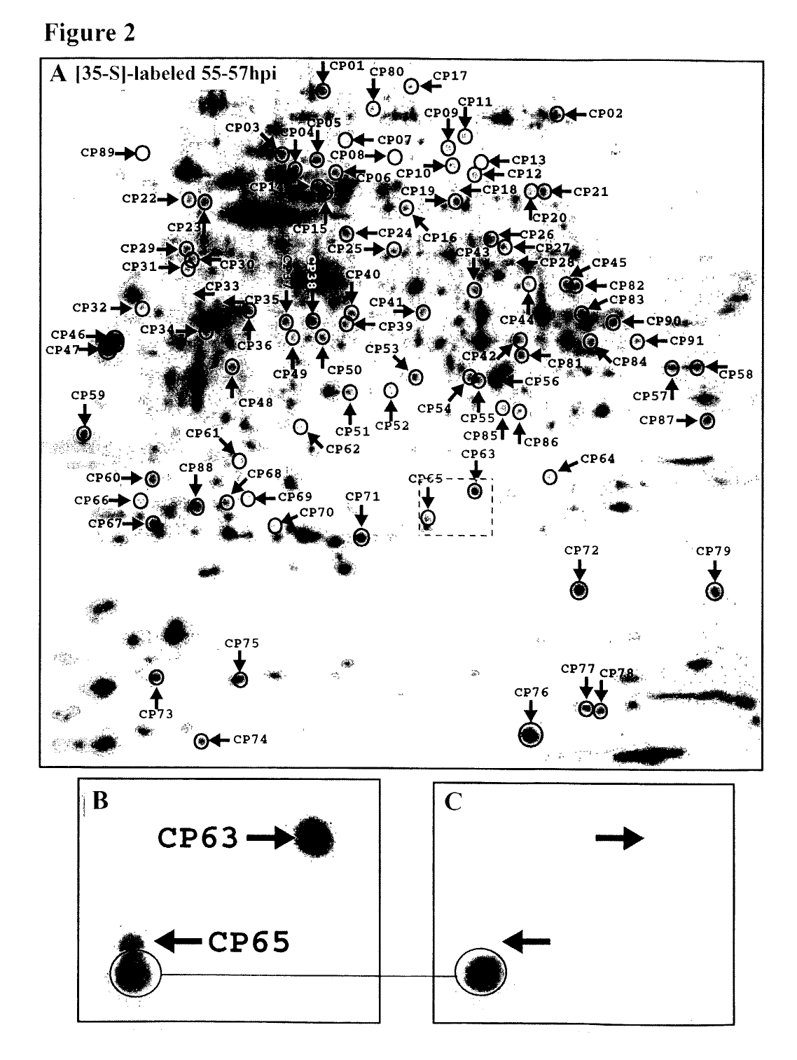

55-57 hpi: enlargements of the gel image shown in FIG. 2A of C. pneumoniae VR1310 proteins from whole lysates of cells [35S]-labelled from 55-57 h.p.i., harvested immediately after labelling and separated by 2D-PAGE using non-linear pH 3-10 Immobiline Drystrip.

Purified EB: Corresponding regions from a gel image (non-linear pH 3-10 Immobiline Drystrip) of proteins from bacteria labelled at two hour periods throughout the developmental cycle ie. 6, 12, 24, 36, 42, 48, 54, 60 h.p.i. and purified as EB at 72 h.p.i.

34-36 hpi: Corresponding regions from a gel image (linear pH 4-7 Immobiline Drystrip) of C. pneumoniae VR1310 proteins from whole lysates of infected cells labelled at 34-36 h.p.i. and harvested immediately after labelling.

RB 36 hpi: Corresponding regions from a gel image (linear pH 4-7 Immobiline Drystrip) of C. pneumoniae VR1310 proteins from bacteria purified as RB at 36 h.p.i immediately after labelling from 34-36 h.p.i.

In A-F the secretion candidates CP34, CP37, CP46, CP47, CP52, CP63 and CP75 have been encircled.

G shows a region from the parent images of the regions in A-F that contains no secretion candidates.

### DETAILED DESCRIPTION OF THE INVENTION

Comparison of 2D-PAGE Protein Profiles from Whole Cell Lysates and Purified Bacteria

Proteins, which are present in whole infected cells but absent from purified bacteria have potentially been secreted from the bacteria, e.g. Chlamydia. Thus, an initial method of the invention is a comparison of 2D-PAGE protein profiles of [35S]-labelled Chlamydia proteins from whole cell lysates of infected cells labelled at different time points of the developmental cycle to 2D-PAGE protein profiles of purified bacteria [35S]-labelled at corresponding time points. This method provides the detection of several proteins, which are clearly present in the protein profile of whole cell lysates, but only faintly detectable or absent in the protein profile of purified bacteria.

From a total of approximately 600 protein spots visualized in whole cell lysates at 22-24 h.p.i. by high resolution 2D-PAGE (IPG), these studies elucidated the existence of 77 C. trachomatis D proteins, of which the intensity is significantly reduced in elementary bodies (EB). Similarly, 91 proteins had significantly reduced intensities in C. pneumoniae VR1310, when comparing whole cell lysates from labelling 55-57 h.p.i. to purified EB. The detected and annotated proteins have the Mw and pI described for protein no. DT1-DT77 for C. trachomatis D, as listed in Table I, and CP1-CP91 for C. pneumoniae as listed in Table II.

This method gives an overview of potentially secreted proteins necessary for further investigations. The examples shows comparison of whole cell lysates to purified EB labelled either at i) time points corresponding to labelling of the whole cell lysates (C. trachomatis) FIG. 1 or 2) at time points throughout the entire developmental cycle (C. pneumoniae, FIG. 2).

Purification of RB allows the discrimination between secreted proteins and RB-specific proteins. Protein profiles of whole lysates of infected cells can be compared to protein profiles of RB purified at the same point in time to identify secreted proteins. In this approach RB specific proteins will not be detected as false positives. Proteins synthesized and secreted at the investigated point in time will be detected. Proteins may be synthesized and secreted at other points in time.

The method also includes detection of proteins secreted immediately after infection, which may have been synthesised in the preceding developmental cycle. These proteins are visualised by infection with EB labelled in the preceding developmental cycle followed by 2D-PAGE of total cell lysates. At an early stage of the developmental cycle before EB differentiate to RB, host cell cytoplasm is obtained by Saponin penetration of the cell membrane [30, 31]. This is only possible because there at this time will be no contamination of Chlamydia proteins from disrupting RB.

Identification of Vaccine Candidates

The vaccine candidate proteins cut out from preparative 2D-gels are identified through advanced mass spectrometric methods.

The excised spots are digested with an enzyme such as trypsin, which generates a number of tryptic peptides. By means of MALDI-MS or other approaches the masses of these peptides are determined with an accuracy of better than 100 parts per million (ppm). Obtained masses are matched to theoretical tryptic cleavage products of all proteins present in databases using the MS-Fit or Peptidesearch software allowing the identification of the analyzed protein on a statistical basis.

When protein spots are cut out from gels loaded with whole cell lysates, contaminating host cell proteins may be located at the same positions as bacteria proteins and as a further complication one spot may contain more than one bacteria protein. To avoid interference from contaminants that may lead to unambiguous identifications, ESI-Q-TOF or post source decay (PSD) may e.g. be used to obtain sequence information of the bacteria protein(s) if necessary.

The method includes proteins, which are identified by mass spectrometry as indicated by examples from C. trachomatis D or C. pneumonia VR1310 in Table III (A and B, respectively).

Accordingly, the invention relates in a first aspect to a method for identifying proteins secreted from an intracellular bacteria, comprising the following steps:

1) infecting host cells by the intracellular bacteria,

2) labelling the intracellular bacteria present in the infected cells,

3) preparing

a) whole cell lysates of the infected cells

b) purified and lysed bacteria from the infected cells,

4) comparing 2D-gel electrophoresis protein profiles of i) the whole cell lysates from step 3a) with ii) the purified and lysed bacteria from step 3b),

5) detecting protein spots from step 4) which are present in the whole cell lysates but absent or present in significantly reduced amount in the purified bacteria,

6) identifying the proteins in the spots selected in step 5).

Pulse/Chase of Vaccine Candidates

The object of this method is to detect secreted proteins, which are degraded in the host cell. In order to estimate the time for which the identified vaccine candidate proteins are present inside the host cell a series of pulse/chase studies are performed. The turnover time of [35S]-labelled proteins is monitored on 2D-gels by chasing the proteins for various periods after labelling. The turnover time provides valuable information on how fast the proteins are degraded, thus how long they are present inside the infected cell.

The method provides estimated turnover times of potentially secreted C. trachomatis proteins as exemplified in Table I.

In this alternative aspect of the invention it relates, accordingly, to a method for identifying proteins secreted from an intracellular bacteria, comprising the following steps:

1) infecting host cells by the intracellular bacteria,

2) pulse labelling of the intracellular bacteria present in the infected cells,

3) preparing whole cell lysates of the infected cells after different periods of chase following step 2),

4) comparing 2D-gel electrophoresis protein profiles of the whole cell lysates prepared after different period of chase from step 3),

5) detecting protein spots from step 4) which are present in decreasing amount as chasing periods increase in step 3),

6) identifying the proteins in the spots selected in step 5).

Pulse Chase in Combination With Proteasome Inhibitors

In order to limit the number of candidates suitable for a vaccine, the invention includes proteasome inhibitor methods in combination with pulse chase studies. These experiments provide an excellent tool for monitoring the effect of the host cell proteasome on the turnover time of secreted Chlamydial proteins.

Immunogenic proteins, which are presented on the surface of eukaryotic cell as MHC-class I antigens must be ubiquitinylated and cleaved by proteolysis in a multi-catalytic protein complex, the proteasome. The proteasome cleaves immunogenic proteins into peptides of a typical length of 8-10 amino acids. These peptides are transported to the ER (endoplasmatic reticulum), where they are bound to a heterodimeric MHC class I molecule, and the MHC-antigen complex is subsequently transported to the surface of cells. On the surface of the cell the MHC-antigen complex will be recognizable by specific receptors on cytotoxic T-lymphocytes (reviewed in Rock and Goldberg [6.]).

It is possible to inhibit the activity of the eukaryotic proteasome and prevent MHC class I presentation by adding cell-permeable proteasome inhibitors such as peptide aldehydes to cell cultures (Rock et al. 1994) [36.]. Chlamydia proteins for which the turnover time is prolonged by the addition of proteasome inhibitors are likely to be secreted from the bacteria and subsequently processed by the proteasome. In addition, this part of the invention will allow the detection of Chlamydial proteins, which are degraded in the proteasome very shortly after their release into the host cell and therefore only detectable in the presence of proteasome inhibitors.

The invention comprises C. trachomatis D and C. pneumonia VR1310 vaccine candidates, which are affected by proteasome inhibitors.

The proteins DT1, DT2, DT3, DT5, DT9, DT10, DT11, DT13, DT14, DT36, DT47, DT59, DT60, DT61, DT62 (as set out in Table IV below) are examples of C. trachomatis D vaccine candidates for which the turnover time is prolonged by addition of proteasome inhibitors during the chase period.

The invention also provides a method for purifying RB, which before harvest are treated with proteasom inhibitors during a labelling period. A comparison of proteasome treated whole cell lysates labelled at the same time as proteasome inhibitor treated purified RB, will elucidate further, which proteins are secreted to the host cell cytoplasm and degraded in the proteasome. In addition, the host cell lines in these experiments can be genetically altered to overexpress genes, which are pivotal in the processing of MHC class I restricted T-cell epitopes [38, 39, 40] (Sijts, 2000, Van Hall, 2000, Shockett, 1995). By the use of such cell lines the effect of proteasome inhibitors will be more pronounced. The invention therefore also comprises the use of commercially available host cell lines, which has been genetically modified in genes, which are involved in MHC-class I antigen presentation.

Accordingly, the invention relates in another alternative aspect to a method for identifying proteins secreted from an intracellular bacteria, comprising the following steps:

1) infecting host cells by the intracellular bacteria,

2) cultivating the host cells in the presence and in the absence of a proteasome inhibitor, respectively,

3) labelling the intracellular bacteria present in the infected cells cultivated in the presence and in the absence of a proteasome inhibitor, respectively,

4) preparing whole cell lysates of the infected cells,

5) comparing 2D-gel electrophoresis protein profiles of the whole cell lysates of the infected cells cultivated in the presence and in the absence of a proteasome inhibitor, respectively,

6) detecting protein spots from step 5) which are present in the whole cell lysates cultivated in the presence of a proteasome inhibitor, but absent or present in significantly reduced amount in the whole cell lysates cultivated in the absence of a proteasome inhibitor,

7) identifying the proteins in the spots selected in step 6).

Generation of Polyclonal Antibodies

The invention provides polyclonal antibodies, which are specific for vaccine candidates. The gene encoding the vaccine candidate protein is cloned using e.g. the ligation independent cloning (LIC)-system. Expressed fusion proteins encompassing the sequence of the vaccine candidate are used to immunize rabbits in order to obtain sera containing vaccine candidate specific poly-clonal antibodies (PAbs). The invention uses the PAbs in 2D-PAGE immuno blotting in order to confirm the correct specificity of the antibody by co-localization or to identify unrecognized isoforms of vaccine candidates. The invention provides verification/identification of vaccine candidates by immunoblotting and co-localization as exemplified in Table III. The invention uses the PAbs to determine the sub-cellular localization of vaccine candidates by means of e.g. indirect immunofluorescence microscopy.

The invention, therefore, also provides one of the alternative methods above which method further comprises the following steps:

1) obtaining antibodies against proteins from said intracellular bacteria, identified according to any of the above methods,

2) 2D-PAGE immunoblotting on whole cell lysates of cells infected with the bacteria using antibodies obtained in step 1),

3) detecting protein spots reacting in step 2),

4) identifying the proteins in the spots selected in step 3).

Combinations of the four alternative methods are also part of the invention.

In preferred embodiments of the methods of the invention the labelling of the intracellular bacteria is done by radioactive means, such as [35S]cysteine, [35S]methionine, [14C]labelled amino acids or combinations thereof.

The method for identifying the proteins in the selected protein spots may be based on Edman degradation or any mass spectrometric method, such as MALDI TOF MS (Matrix-Assisted Laser Desorption/Ionisation Time-Of-Flight Mass Spectrometry), ESI Q-TOF MS (Electrospray Ionisation Quadrupole Time-Of-Flight Mass Spectrometry), PSD-MALDI MS (Post Source Decay MALDI Mass Spectrometry) or combinations of such methods. Further, the proteins may, prior to identification, be subjected to cleavage by chemical methods, such as cyanogen bromide treatment or hydroxylamine treatment, or by enzymatic methods with any suitable enzymes, such as trypsin, slymotrypsin, chymotrypsin, or pepsin, or combinations thereof.

The intracellular bacteria may be a facultative intracellular or obligate intracellular bacteria, and bacteria from the Genus Chlamydia, such as C. pneumoniae, C. trachomatis, C. psitacci or C. pecorum, including any specific serovar or strain of these, are particularly interesting. However, other intracellular bacteria, such as Salmonella, Shigella, Yersinia or Listeria, are interesting, too, in connection with the present invention.

The host cells to be used according to the invention, may be common host cells known in the art, such as an immortalized cell line, e.g. HeLa, Hep2, McCoy or U937, a primary cell line obtained from mammalian donors or by autopsy, a genetically modified cell line, or an organ cell culture, or even other cells wherein the bacteria can grow. The host cells may be treated with IFN-γ prior to or during infection with the intracellular bacteria and/or may have been genetically modified to over-express or suppress genes which are recognized as being relevant in context of Chlamydial vaccine development.

When the method of the invention uses one or more proteasome inhibitor, any known inhibitor, such as MG132, MG262, MG 115, epoxymycin, PSI and clasto-Lactacystin-β-lactone, are relevant to use.

The methods of the invention are particularly interesting for identification of proteins, which either in their full length or as immunogenic fragments thereof are suitable for inclusion in immunogenic compositions and/or diagnostic purposes, especially such proteins, which comprises T-cell epitopes being candidates for presentation as MHC-class I or II, and more preferable class I, restricted antigens suitable for inclusion in immunogenic compositions.

Accordingly, in another important aspect of the invention it relates to a protein identifiable by any of the claimed methods or an immunogenic fragment thereof, and preferable such proteins and fragments, which are applicable for inclusion in immunogenic compositions and/or diagnostic purposes.

The proteins of the invention may be proteins, which are secreted from C. trachomatis and C. pneumoniae. Such proteins are e.g. those characterized as DT1-77 as given in Table I as well as CP1-CP91 as given in Table II, respectively, having the pI and Mw values given in the Tables I and II, respectively, determined with an average error of +/−10%, and immunogenic fragments thereof.

TABLE I

| Protein spot | Pi | Mw |
|---|---|---|
| DT1 | 4.45 | 23.5 |
| DT2 | 4.55 | 23.5 |
| DT3 | 4.55 | 34.5 |
| DT4 | 4.75 | 36.1 |
| DT5 | 4.83 | 11.4 |
| DT6 | 9.3 | 9.27 |
| DT7 | 4.85-4.9 | 24.8 |
| DT8 | 5.1 | 7.8 |
| DT9 | 4.73 | 23.7 |
| DT10 | 4.8 | 23.7 |
| DT11 | 4.85 | 23.7 |
| DT12 | 4.93 | 23.7 |
| DT13 | 6.05 | 24.3 |
| DT14 | 6.2 | 27.5 |
| DT15 | 6.1 | 32.4 |
| DT16 | 5.98 | 39 |
| DT17 | 6.28 | 55.2 |
| DT18 | 6.1 | 41.1 |
| DT19 | 6.1 | 47.9 |
| DT20 | 7.4 | 37.6 |
| DT21 | 7.7 | 34.7 |
| DT22 | 8.2 | 22.4 |
| DT23 | 4.83 | 30.4 |
| DT24 | 5 | 29.5 |
| DT25 | 5 | 12.6 |
| DT26 | 4.7 | 10.9 |
| DT27 | 5.15 | 13.5 |
| DT28 | 5.7 | 31.9 |
| DT29 | 4.97 | 54.8 |
| DT30 | 5.86 | 36 |
| DT31 | 5.78 | 36.2 |
| DT32 | 6.4 | 10.4 |
| DT33 | 6.3 | 13.3 |
| DT34 | 9.5 | 32.4 |
| DT35 | 8 | 46.5 |
| DT36 | 7.49 | 40.6 |
| DT37 | 7.15 | 37.6 |
| DT38 | 7.24 | 34.5 |
| DT39 | 7.44 | 46.5 |
| DT40 | 6.4 | 67.2 |
| DT41 | 5.04 | 56.4 |
| DT42 | 8.5 | 32.9 |
| DT43 | 8.5 | 30.5 |
| DT44 | 8.66 | 42.6 |
| DT45 | 8.85 | 43.1 |
| DT46 | 4.4 | 87.6 |
| DT47 | 5.4 | 41 |
| DT48 | 7.36 | 24.2 |
| DT49 | 9.25 | 47.4 |
| DT50 | 5.0 | 94 |
| DT51 | 5.35 | 100.5 |
| DT52 | 5.41 | 59.7 |
| DT53 | 5.97 | 23 |
| DT54 | 6.12 | 25.5 |
| DT55 | 5.34 | 36.4 |
| DT56 | 4.88 | 10.5 |
| DT57 | 4.87 | 18.5 |
| DT58 | 6.14 | 97 |
| DT59 | 4.5 | 19.7 |
| DT60 | 5.5 | 40.9 |
| DT61 | 5.5 | 39.9 |
| DT62 | 5.98 | 41.1 |
| DT63 | 6.9 | 46.8 |
| DT64 | 5.5 | 34.5 |
| DT65 | 4.5 | 68.2 |
| DT66 | 4.35 | 57.6 |
| DT67 | 6.13 | 66.5 |
| DT68 | 6 | 62.9 |
| DT69 | 5.85 | 65.6 |
| DT70 | 5.72 | 70.4 |
| DT71 | 5.5 | 44 |
| DT72 | 5.85 | 10.2 |
| DT73 | 4.45 | 30.5 |
| DT74 | 5.02 | 48.2 |
| DT75 | 4.37 | 21.9 |
| DT76 | 5.14 | 23.3 |
| DT77 | 5.64 | 23.0 |

List of potentially secreted proteins from C. trachomatis D present in whole cell lysates at 24 h.p.i., but significantly reduced in EB and their estimated pI/Mw, +/−10% average error.

TABLE II

| Name | Pi | Mw |
|---|---|---|
| CP01 | 5.5 | 100.4 |
| CP02 | 6.7 | 91.0 |
| CP03 | 5.3 | 75.2 |
| CP04 | 5.4 | 68.7 |
| CP05 | 5.4 | 72.8 |
| CP06 | 5.5 | 68.6 |
| CP07 | 5.6 | 80.5 |
| CP08 | 5.7 | 74.1 |
| CP09 | 6.0 | 77.8 |
| CP10 | 6.0 | 71.2 |
| CP11 | 6.1 | 82.6 |
| CP12 | 6.2 | 68.4 |
| CP13 | 6.2 | 72.0 |
| CP14 | 5.4 | 64.8 |
| CP15 | 5.5 | 63.7 |
| CP16 | 5.8 | 61.1 |
| CP17 | 5.8 | 102.4 |
| CP18 | 6.1 | 63.6 |
| CP19 | 6.1 | 61.0 |
| CP20 | 6.5 | 64.0 |
| CP21 | 6.6 | 63.7 |
| CP22 | 5.0 | 60.9 |
| CP23 | 5.0 | 60.5 |
| CP24 | 5.6 | 60.4 |
| CP25 | 5.7 | 50.2 |
| CP26 | 6.3 | 57.0 |
| CP27 | 6.3 | 52.8 |
| CP28 | 6.4 | 48.5 |
| CP29 | 4.9 | 50.6 |
| CP30 | 5.0 | 48.6 |
| CP31 | 4.9 | 473 |
| CP32 | 4.7 | 42.1 |
| CP33 | 4.9 | 439 |
| CP34 | 5.0 | 39.3 |
| CP35 | 5.1 | 42.9 |
| CP36 | 5.2 | 42.1 |
| CP37 | 5.3 | 40.7 |
| CP38 | 5.4 | 40.7 |
| CP39 | 5.6 | 40.3 |
| CP40 | 5.6 | 41.6 |
| CP41 | 5.9 | 41.8 |
| CP42 | 6.4 | 38.4 |
| CP43 | 6.2 | 44.3 |
| CP44 | 6.5 | 45.3 |
| CP45 | 6.8 | 45.3 |
| CP46 | 4.6 | 38.6 |
| CP47 | 4.6 | 37.8 |
| CP48 | 5.1 | 35.8 |
| CP49 | 5.3 | 38.9 |
| CP50 | 5.5 | 38.9 |
| CP51 | 5.6 | 33.6 |
| CP52 | 5.7 | 33.7 |
| CP53 | 5.9 | 34.9 |
| CP54 | 6.2 | 34.8 |
| CP55 | 6.2 | 34.7 |
| CP56 | 6.3 | 34.8 |
| CP57 | 8.3 | 36.0 |
| CP58 | 8.7 | 36.1 |
| CP59 | 4.5 | 29.7 |
| CP60 | 4.8 | 26.0 |
| CP61 | 5.2 | 27.6 |
| CP62 | 5.4 | 30.6 |
| CP63 | 6.2 | 25.2 |
| CP64 | 6.6 | 26.3 |
| CP65 | 5.9 | 22.8 |
| CP66 | 4.7 | 24.2 |
| CP67 | 4.8 | 22.4 |
| CP68 | 5.1 | 24.1 |
| CP69 | 5.2 | 24.3 |
| CP70 | 5.3 | 22.3 |
| CP71 | 5.6 | 21.4 |
| CP72 | 6.9 | 17.8 |
| CP73 | 4.8 | 12.0 |
| CP74 | 5.0 | 8.9 |
| CP75 | 5.1 | 11.9 |
| CP76 | 6.5 | 9.3 |
| CP77 | 7.0 | 10.5 |
| CP78 | 7.2 | 10.4 |
| CP79 | 8.7 | 13.0 |
| CP80 | 5.7 | 93.3 |
| CP81 | 6.4 | 37.2 |
| CP82 | 6.9 | 45.0 |
| CP83 | 7.0 | 41.6 |
| CP84 | 7.1 | 38.6 |
| CP85 | 6.3 | 32.2 |
| CP86 | 6.4 | 32.0 |
| CP87 | 8.8 | 31.3 |
| CP88 | 5.0 | 23.8 |
| CP89 | 4.7 | 73.0 |
| CP90 | 7.4 | 40.1 |
| CP91 | 7.8 | 37.7 |

List of potentially secreted proteins from C. pneumoniae present in whole cell lysates at 55 h.p.i but significantly reduced in EB and their estimated pI/Mw, +/−10% average error.

More preferred proteins of the inventions are C. trachomatis proteins, such as those identified by the corresponding gene number in the Chlamydia Genome project as CT017 (gene name CT017), CT044 (gene name ssp), CT243 (gene name IpxD), CT263 (gene name CT263), CT265 (gene name accA), CT286 (gene name cipC), CT292 (gene name dut), CT407 (gene name dksA), CT446 (gene name euo), CT460 (gene name SWIB), CT541 (gene name mip), CT610 (gene name CT610), CT650 (gene name recA), CT655 (gene name kdsA), CT668 (gene name CT668), CT691 (gene name CT691), CT734 (gene name CT734), CT783 (gene name CT783), CT858 (gene name CT858), CT875 (gene name CT875), or ORF5 (gene name ORF5), or by the protein name DT8 as given in Table IIIA, and C. pneumoniae proteins, such as those identified by the corresponding gene number as CPN0152 (gene name CPN0152), CPN0702, CPN0705 (gene name CPN0705), CPN0711 (gene name CPN0711), CPN0998 (gene name ftsH), CPN0104 (gene name CPN0104), CPN0495 (gene name aspC), CPN0684 (gene name parB), CPN0796 (gene name CPN0796), CPN0414 (gene name accA), CPN1016 (gene name CPN1016), CPN1040 (gene name CPN1040), CPN0079 (gene name RI10), CPN0534 (gene name dksA), CPN0619 (gene name ndk), CPN0711 (gene name CPN0711), CPN0628 (gene name rs13), CPN0926 (gene name CPN0926), CPN1063 (gene name tpiS), or CPN0302 (gene name IpxD) as given in Table IIIB, and immunogenic fragments thereof.

TABLE III A

| Protein spot | Gene | | Methods of | | |
|---|---|---|---|---|---|
| nr | number* | Gene name | identification | pl | Mw |
| DT1 | CT668 | CT668 | M, Q, I | 4.45 | 23.5 |
| DT2 | CT668 | CT668 | M, Q, I | 4.55 | 23.5 |
| DT3 | CT783 | CT783 | M, Q, I | 4.55 | 34.5 |
| DT4 | CT858 | CT858 | M, Q, I | 4.75 | 36.1 |
| DT48 | CT858 | CT858 | M, I | 7.36 | 24.2 |
| DT7 | CT610 | CT610 | M, E, I | 4.85-4.9 | 24.8 |
| DT9 | CT610 | CT610 | I | 4.73 | 23.7 |
| DT10 | CT610 | CT610 | I | 4.8 | 23.7 |
| DT11 | CT610 | CT610 | I | 4.85 | 23.7 |
| DT12 | CT610 | CT610 | I | 4.93 | 23.7 |
| DT8 | None | DT8* | Q, I | 5.1 | 7.8 |
| DT6 | CT460 | SWIB | M | 9.3 | 9.27 |
| DT14 | ORF5 | ORF5 | M | 6.2 | 27.5 |
| DT22 | CT446 | euo | Q | 8.2 | 22.4 |
| DT23 | CT541 | mip | M | 4.83 | 30.4 |
| DT24 | CT541 | mip | M | 5 | 29.5 |
| DT25 | CT407 | dksA | M, Q | 5 | 12.6 |
| DT26 | CT734 | CT734 | Q, I | 4.7 | 10.9 |
| DT27 | CT292 | dut | M, Q | 5.15 | 13.5 |
| DT28 | CT655 | kdsA | M, E | 5.7 | 31.9 |
| DT30 | CT265 | accA | M | 5.86 | 36 |
| DT35 | CT017 | CT017 | M | 8 | 46.5 |
| DT39 | CT017 | CT017 | M | 7.44 | 46.5 |
| DT36 | CT243 | IpxD | M | 7.49 | 40.6 |
| DT37 | CT650 | recA | M, E | 7.15 | 37.6 |
| DT57 | CT044 | ssp | M | 4.87 | 18.5 |
| DT58 | CT286 | cipC | M | 6.14 | 97 |
| DT69 | CT875 | CT875 | M | 5.85 | 65.6 |
| DT76 | CT691 | CT691 | Q | 5.14 | 23.3 |
| DT77 | CT263 | CT263 | Q | 5.64 | 23.0 |

TABLE III B

| Protein spot | | | Methods of | | |
|---|---|---|---|---|---|
| nr | Gene number | Gene name | identification | pl | Mw |
| CP34 | CPN1016 | CPN1016 | I | 5.0 | 39.3 |
| CP37 | CPN0998 | ftsH | M | 5.3 | 40.7 |
| CP42 | CPN0104 | CPN0104 | M | 6.4 | 38.4 |
| CP46 | CPN0796 | CPN0796 | Q | 4.6 | 38.6 |
| CP47 | CPN0705 | CPN0705 | M | 4.6 | 37.8 |
| CP50 | CPN0495 | aspC | M | 5.5 | 38.9 |
| CP52 | CPN0152 | CPN0152 | M | 5.7 | 33.7 |
| CP55 | CPN0684 | parB | M | 6.2 | 34.7 |
| CP56 | CPN0414 | accA | M | 6.3 | 34.8 |
| CP63 | CPN1016 | CPN1016 | M | 6.2 | 25.2 |
| CP71 | CPN1040 | CPN1040 | M | 5.6 | 21.4 |
| CP72 | CPN0079 | rl10 | M | 4.8 | 12.0 |
| CP73 | CPN0534 | dksA | M | 5.0 | 8.9 |
| CP75 | CPN0619 | ndk | M | 5.1 | 11.9 |
| CP76 | CPN0711 | CPN0711 | M | 6.5 | 9.3 |
| CP78 | CPN0628 | rsl3 | M | 7.2 | 10.4 |
| CP79 | CPN0926 | CPN0926 | M | 8.7 | 13.0 |
| CP88 | CPN1063 | tpiS | M | 5.0 | 23.8 |
| CP91 | CPN0302 | IpxD | M | 7.8 | 37.7 |

List of examples of identified A: C. trachomatis D and B: C. pneumoniae vaccine candidates. M: MALDI-MS, Q: ESI-Q-TOF MS, P: PSD-MALDI MS, I: Western blotting.

*: DT8 represents an expressed protein encoded by a novel open reading frame, which is not annotated in the C. trachomatis D genome.

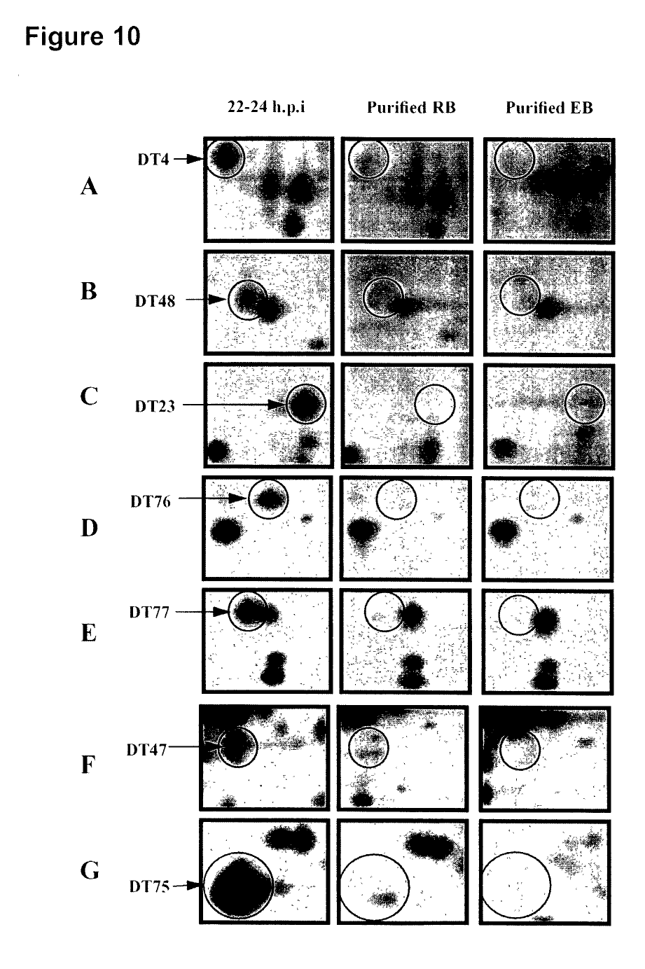

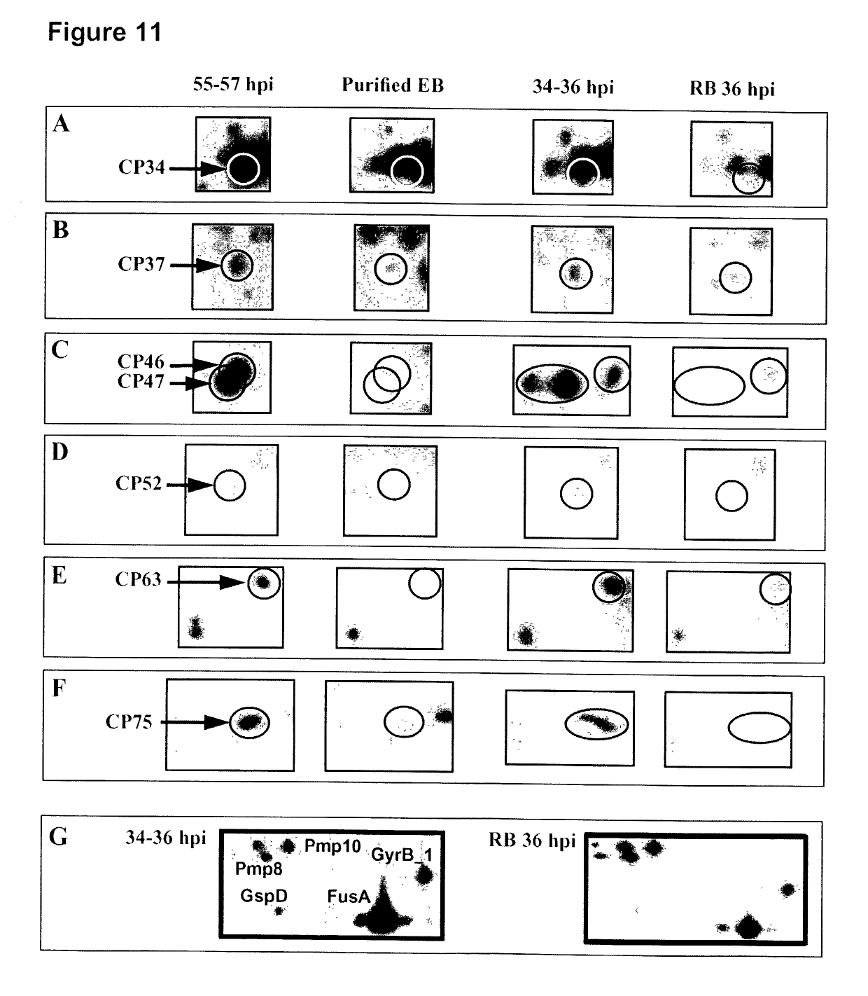

The proteins DT4, DT23, DT47, DT48, DT75, DT76, and DT 77 shown in FIG. 10, as well as the proteins CP34, CP37, CP46, CP47, CP52, CP63, and CP75 shown in FIG. 11 are of particular relevance.

Also preferred proteins of the invention are C. trachomatis proteins, which are proteins that have a prolonged turnover time in the presence of proteasome inhibitors, being characterised by having the pI and Mw characteristics of one of the proteins DT1, DT2, DT3, DT5, DT9, DT10, DT11, DT13, DT14, DT17, DT47, DT59, DT60, DT61 or DT62 as given in Table IV, determined with an average error of +/−10%. Proteins from C. pneumoniae, which are regulated by proteasome inhibitors in the same way, are also preferred embodiments of the invention.

TABLE IV

| | | | | | | | | |
|---|---|---|---|---|---|---|---|---|
| | | | | | | | | Affected |
| | | | | Affected | Affected | Affected | Affected | by B- |
| Spot | Gene | | | by | by | by | by | clasto |
| nr. | name | pl | Mw | MG115 | MG132 | PSI | epoximicin | lactacystin |

| | | | | | | | |
|---|---|---|---|---|---|---|---|
| DT1 | CT668 | 4.45 | 23.5 | + | + | | |
| DT2 | CT668 | 4.55 | 23.5 | + | + | | |
| DT3 | CT783 | 4.55 | 34.5 | + | + | | |
| DT5 | | 4.83 | 11.4 | + | + | | |
| DT9 | CT610 | 4.73 | 23.7 | + | + | | |
| DT10 | CT610 | 4.8 | 23.7 | + | + | | |
| DT11 | CT610 | 4.85 | 23.7 | + | + | | |
| DT13 | | 6.05 | 24.3 | + | + | + | |
| DT14 | ORF5 | 6.2 | 27.5 | + | + | | |
| DT17 | | 6.28 | 55.2 | + | + | | |
| DT47 | | 5.4 | 41 | + | + | + | + |
| DT59 | | 4.5 | 19.7 | + | + | | |
| DT60 | | 5.5 | 40.9 | + | + | + | + |
| DT61 | | 5.5 | 39.9 | + | + | + | + |
| DT62 | | 5.98 | 41.1 | + | + | + | + |

List of examples of identified C. trachomatis D proteins, which turnover times is prolonged by treatment with different proteasome inhibitors during labelling and chase.

Another preferred protein of the invention is a Chlamydia trachomatis polypeptide DT8, which comprises the sequence SEQ ID NO:1 as defined in the claims and immunogenic fragments thereof.

In other preferred embodiments of this aspect of the invention the proteins have at least 40% sequence identity, preferable at least 60%, more preferable at least 70%, even more preferable at least 80%, further more preferable 90%, and most preferable at least 95% sequence identity to the proteins or fragments thereof of the invention, or it comprises at least 7 consecutive amino acids of the proteins of the invention.

In a further aspect of the invention it relates to a nucleic acid compound, which comprises a sequence that encodes a protein or an immunogenic fragment thereof according to the invention.

A preferred nucleic acid compound is one which comprises a sequence (SEQ ID NO:2) that encodes a polypeptide DT8, which comprises the sequence SEQ ID NO:1.

In yet other aspects the invention relates to a vector comprising a nucleic acid compound of the invention, as well as a host cell transformed or transfected with the vector.

The invention also provides the use of a protein or an immunogenic fragment thereof of the invention for the production of antibodies against said protein, a method for producing an antibody against intracellular bacteria, wherein a protein or an immunogenic fragment thereof of the invention are administered to a producing animal, and the antibody is purified there from, as well as an antibody obtainable by this method.

Further, the invention provides in other aspects a pharmaceutical or diagnostic composition comprising the protein or fragment thereof of the invention, an antibody or a nucleic acid compound of the invention, as well as the use of the protein or fragment thereof, the antibody or the nucleic acid compound in the preparation of a diagnostic reagent.

Identification of T-Cell Epitopes from Vaccine Candidates

The invention provides T-cell epitopes, that are likely to be surface expressed as MHC class I antigens and have a T-cell stimulating effect, as predicted by computer based methods from the sequences of the proteins identified in the present invention, or experimentally determined by assays as further described in the examples.

Accordingly, another important aspect of the invention is peptide epitopes that are likely to be surface presented as MHC Class I antigens and have a T-cell stimulating effect. In accordance with this the invention provides a method for identification of T-cell epitopes on secreted proteins from intracellular bacteria, comprising steps, such as computer prediction, MHC class molecule binding assays and/or ELISPOT assays on a protein or an immmunogenic fragment thereof identified by the methods of the invention, as well as the peptide epitopes. As part of this aspect the invention also provides a nucleic acid compound, which comprises a sequence that encodes said peptide epitope, as well as a vector comprising the nucleic acid compound and a host cell transformed with said vector.

Preferred peptide epitopes of the invention comprises 4 to 25 consecutive amino acids, preferably 6 to 15, and even more preferably 7 to 10 amino acids of a protein of the invention.

In a more preferred embodiment of the invention the epitopes comprises 7 to 10 consecutive amino acids of a C. trachomatis or a C. pneumoniae protein.

Another preferred peptide epitope of the invention is an epitope, which comprises 4 to 25 consecutive amino acids of a polypeptide comprising the sequence SEQ ID NO:1, more preferably 6 to 15 and most preferably 7 to 10 amino acids.

Chlamydia trachomatis peptide epitopes, which comprises an amino acid sequence selected form the sequences SEQ ID NO. 3-SEQ ID NO. 45, Chlamydia pneumonia peptide epitopes, which comprises an amino acid sequence selected from the sequences SEQ ID NO. 46-SEQ ID NO. 121, Chlamydia pneumonia peptide epitopes, which comprises an amino acid sequence, selected from the sequences of SEQ ID NO. 122-SEQ. ID NO. 148, as well as and Chlamydia trachomatis peptide epitopes, which comprises an amino acid sequence, selected from the sequences of SEQ ID NO. 149-SEQ. ID NO. 194 are of particular relevance. The identified epitopes of the invention are further characterized in Tables V-VIII.

TABLE V

| Protein ID | Position | Peptide sequence | A2 binding |
|---|---|---|---|
| CT263 | 181 | KLAEAIFPI | 8 |
| CT263 | 170 | FLKNNKVKL | 123 |
| CT263 | 56 | ALSPPPSGY | 210 |
| CT263 | 141 | FIAKQASLV | 210 |
| CT263 | 17 | TLSLFPFSL | 286 |
| CT263 | 147 | SLVACPCSM | 332 |
| CT263 | 6 | LIFADPAEA | 386 |
| CT263 | 4 | LLLIFADPA | 438 |
| CT541 | 4 | ILSWMLMFA | 38 |
| CT541 | 94 | KQMAEVQKA | 89 |
| CT541 | 9 | LMFAVALPI | 122 |
| CT541 | 135 | KLQYRWKE | 221 |
| CT541 | 118 | FLKENKEKA | 222 |
| CT541 | 46 | KLSRTFGHL | 239 |
| CT541 | 223 | SLLIFEVKL | 265 |
| CT541 | 148 | VLSGKPTAL | 352 |
| CT541 | 204 | VLYIHPDLA | 398 |
| CT541 | 54 | LLSRQLSRT | 472 |
| CT691 | 172 | LLQRELMKV | 9 |
| CT691 | 25 | STINVLFPL | 66 |
| CT691 | 15 | PLQAHLELV | 114 |
| CT691 | 6 | SLFGQSPFA | 194 |
| CT691 | 212 | KLAYRVSMT | 251 |
| CT691 | 194 | VLWMQIIKG | 284 |
| CT691 | 29 | VLFPLFSAL | 298 |
| CT691 | 122 | FLQKTVQSF | 468 |
| CT691 | 8 | FGQSPFAPL | 480 |
| CT858 | 85 | VLADFIGGL | 33 |
| CT858 | 177 | RMASLGHKV | 52 |
| CT858 | 92 | GLNDFHAGV | 90 |
| CT858 | 490 | FSCADFFPV | 90 |
| CT858 | 379 | MLTDRPLEL | 101 |
| CT858 | 408 | LLENVDTNV | 121 |
| CT858 | 391 | RMILTQDEV | 132 |
| CT858 | 491 | SCADFFPW | 132 |
| CT858 | 519 | FVFNVQFPN | 132 |
| CT858 | 372 | YLYALLSML | 247 |
| CT858 | 539 | SLAVREHGA | 288 |
| CT858 | 109 | YLPYTVQKS | 350 |
| CT858 | 219 | ATIAPSIRA | 358 |
| CT858 | 140 | LLEVDGAPV | 375 |
| CT858 | 512 | RTAGAGGFV | 384 |
| CT858 | 250 | SLFYSPMVP | 431 |

Predicted Epitopes from Identified C. trachomatis Proteins

TABLE VI

| Protein ID | Position | Peptide sequence | A2 binding |
|---|---|---|---|
| CPN0152 | 6 | FLVSCLFSV | 18 |
| CPN0152 | 135 | YLRDAQTIL | 28 |
| CPN0152 | 237 | LLIRIQDHV | 48 |
| CPN0152 | 100 | KLGRKFAAV | 51 |
| CPN0152 | 266 | LVSRTQQTL | 164 |
| CPN0152 | 10 | CLFSVAIGA | 190 |
| CPN0152 | 222 | GFGPPPIIV | 354 |
| CPN0152 | 249 | SLPTKPYIL | 387 |
| CPN0152 | 240 | RIQDHVTAN | 408 |
| CPN0152 | 15 | AIGASAAPV | 410 |
| CPN0152 | 156 | RLGISGFSL | 444 |
| CPN0619 | 64 | FMVSGPWV | 31 |
| CPN0619 | 73 | LVLEGANAV | 398 |
| CPN0705 | 164 | FVGANLTLV | 24 |
| CPN0705 | 89 | CLAENAFAG | 114 |
| CPN0705 | 233 | KIEEVQTPL | 116 |
| CPN0705 | 211 | ALKGHQLTL | 178 |
| CPN0705 | 190 | QMAEAADLV | 358 |
| CPN0796 | 583 | FMGAHVFAS | 15 |
| CPN0796 | 419 | LLIQHSAKV | 31 |
| CPN0796 | 372 | FLCPFQAPS | 39 |
| CPN0796 | 376 | FQAPSPAPV | 50 |
| CPN0796 | 211 | AMNACVNGI | 86 |
| CPN0796 | 548 | FMGIQVLHL | 112 |
| CPN0796 | 74 | RHAAQATGV | 134 |
| CPN0796 | 328 | FQYADGQMV | 148 |
| CPN0796 | 618 | SVSAMGNFV | 212 |
| CPN0796 | 460 | FLSYRSQVH | 214 |
| CPN0796 | 53 | FLLTAIPGS | 218 |
| CPN0796 | 38 | VLTPWIYRK | 219 |
| CPN0796 | 656 | SWMNQQPL | 221 |
| CPN0796 | 408 | SLKNSQQQL | 279 |
| CPN0796 | 162 | MLPDTLDSV | 284 |
| CPN0796 | 511 | ALPYTEQGL | 295 |
| CPN0796 | 523 | VLSGFGGQV | 399 |
| CPN0998 | 22 | LLFGWFGV | 8 |
| CPN0998 | 174 | SLQERYPTL | 29 |
| CPN0998 | 416 | MLLKGQNKV | 33 |
| CPN0998 | 379 | FTFLPIILV | 53 |
| CPN0998 | 754 | FLGDISSGA | 56 |
| CPN0998 | 36 | FLAGKKARV | 66 |
| CPN0998 | 824 | LLDAAYQRA | 66 |
| CPN0998 | 374 | YLGYLFTFL | 78 |
| CPN0998 | 377 | YLFTFLPII | 109 |
| CPN0998 | 717 | SLGATHFLP | 124 |
| CPN0998 | 96 | ELIDQGHRL | 134 |
| CPN0998 | 381 | FLPIILVLL | 197 |
| CPN0998 | 386 | LVLLFVYLV | 219 |
| CPN0998 | 161 | VTGPATPQL | 223 |
| CPN0998 | 319 | SLEKQDPEV | 224 |
| CPN0998 | 567 | ILMAATNRP | 236 |
| CPN0998 | 230 | LTQETDTEA | 237 |
| CPN0998 | 823 | MLLDAAYQR | 238 |
| CPN0998 | 639 | LLNEAALLA | 254 |
| CPN0998 | 736 | ELYDQLAVL | 256 |
| CPN0998 | 199 | LIGKYLSPV | 294 |
| CPN0998 | 454 | SLGGRIPKG | 303 |
| CPN0998 | 781 | GMSPQLGNV | 306 |
| CPN0998 | 645 | LLAARKDRT | 315 |
| CPN0998 | 424 | VTFADVAGI | 427 |
| CPN0998 | 154 | VLTEPLVVT | 439 |
| CPN0998 | 66 | KIALNDNLV | 470 |
| CPN1016 | 5 | KLGAIVFGL | 7 |
| CPN1016 | 135 | YLGDEILEV | 34 |
| CPN1016 | 284 | FLPTFGPIL | 99 |
| CPN1016 | 439 | SLQNFSQSV | 108 |
| CPN1016 | 414 | FTDEQAVAV | 145 |
| CPN1016 | 92 | SLNDYHAGI | 164 |
| CPN1016 | 392 | RMIFTQDEV | 175 |
| CPN1016 | 64 | TQQARLQLV | 294 |
| CPN1016 | 217 | SLVAPLIPE | 312 |
| CPN1016 | 255 | YMVPYFWEE | 358 |
| CPN1016 | 576 | YVEAVKTIV | 389 |
| CPN1016 | 395 | FTQDEVSSA | 444 |
| CPN1016 | 516 | GAGGFVFQV | 491 |
| CPN1016 | 464 | LLGFAQVRP | 498 |

Predicted Epitopes from Identified C. pneumoniae Proteins

TABLE VII

| Protein ID | Position | Peptide sequence | A2 binding |
|---|---|---|---|
| CPN0412 (CT263) | 186 | RLEEVSQKL | 80 |
| CPN0412 (CT263) | 103 | LTTDTPPVL | 103 |
| CPN0412 (CT263) | 147 | KLLDMEGYA | 167 |
| CPN0412 (CT263) | 110 | VLSEDPPYI | 183 |
| CPN0412 (CT263) | 62 | ALQSYCQAY | 215 |
| CPN0412 (CT263) | 193 | KLTQTLVEL | 248 |
| CPN0412 (CT263) | 81 | FVGACSPEI | 267 |
| CPN0412 (CT263) | 102 | NLTTDTPPV | 286 |
| CPN0412 (CT263) | 205 | LMERAIPPK | 410 |
| CPN0661 (CT541) | 103 | KMAEVQKLV | 46 |
| CPN0661 (CT541) | 199 | ALGMQGMKE | 221 |
| CPN0661 (CT541) | 54 | KLSRTFGHL | 239 |
| CPN0661 (CT541) | 232 | LLIFEINLI | 334 |
| CPN0661 (CT541) | 8 | VLATVALAL | 391 |
| CPN0661 (CT541) | 187 | ILLPLGQTI | 396 |
| CPN0661 (CT541) | 212 | VLYIHPDLA | 398 |
| CPN0661 (CT541) | 7 | LVLATVALA | 413 |
| CPN0681 (CT691) | 29 | YMLPIFTAL | 40 |
| CPN0681 (CT691) | 136 | LLHEFNQLL | 66 |
| CPN0681 (CT691) | 172 | VLQRELMQI | 91 |
| CPN0681 (CT691) | 15 | PLQAHLEMV | 169 |
| CPN0681 (CT691) | 6 | RLFGQSPFA | 197 |
| CPN0681 (CT691) | 73 | GLEMPISRA | 223 |
| CPN0681 (CT691) | 212 | KLAHRINMT | 229 |
| CPN0681 (CT691) | 194 | YLWLQVIRR | 322 |
| CPN0681 (CT691) | 135 | TLLHEFNQL | 474 |
| CPN0681 (CT691) | 8 | FGQSPFAPL | 480 |

Predicted Epitopes from C. pneumoniae Homologs to Identified C. trachomatis Proteins

TABLE VIII

| Protein ID | Position | Peptide sequence | A2 binding |
|---|---|---|---|
| CT149 (CPN0152) | 274 | FLGAAPAQM | 17 |
| CT149 (CPN0152) | 237 | FLGIQDHIL | 29 |
| CT149 (CPN0152) | 101 | LLTANGIAV | 31 |
| CT149 (CPN0152) | 248 | SLPRRIPVL | 86 |
| CT149 (CPN0152) | 42 | GLQEHCRGV | 107 |
| CT149 (CPN0152) | 160 | SLGCHTTIH | 170 |
| CT149 (CPN0152) | 307 | ILTHFQSNL | 181 |
| CT149 (CPN0152) | 52 | VLSCGYNLV | 202 |
| CT149 (CPN0152) | 195 | LLKEICATI | 248 |
| CT149 (CPN0152) | 272 | RLFLGAAPA | 318 |
| CT149 (CPN0152) | 141 | ATVAKYPEV | 338 |
| CT149 (CPN0152) | 11 | LLSGSGFAA | 343 |
| CT149 (CPN0152) | 102 | LTANGIAVA | 373 |
| CT149 (CPN0152) | 15 | SGFAAPVEV | 397 |
| CT500 (CPN0619) | 64 | FMJSGPVVV | 20 |
| CT500 (CPN0619) | 103 | ALFGESIGV | 121 |
| CT500 (CPN0619) | 119 | SLENAAIEV | 212 |
| CT500 (CPN0619) | 87 | LMGATNPKE | 313 |
| CT500 (CPN0619) | 31 | RIAAMKMVH | 385 |
| CT671 (CPN0705) | 102 | ALVETPMAV | 13 |
| CT671 (CPN0705) | 167 | FCGANLTLV | 49 |
| CT671 (CPN0705) | 214 | SLKARQLNL | 151 |
| CT671 (CPN0705) | 193 | QLTEATQLV | 239 |
| CT671 (CPN0705) | 127 | DLQWVEQLV | 403 |
| CT671 (CPN0705) | 155 | IVLDNSNTV | 423 |
| CT841 (CPN0998) | 22 | LLFGVIFGV | 9 |
| CT841 (CPN0998) | 415 | LLAKGQNKV | 14 |
| CT841 (CPN0998) | 378 | FTFMPULV | 29 |
| CT841 (CPN0998) | 753 | FLGDVSSGA | 43 |
| CT841 (CPN0998) | 824 | LLDAAYQRA | 66 |
| CT841 (CPN0998) | 780 | GMSDHLGTV | 110 |
| CT841 (CPN0998) | 716 | SLGATHFLP | 124 |
| CT841 (CPN0998) | 170 | NLAALENRV | 153 |
| CT841 (CPN0998) | 376 | YLFTFMPII | 160 |
| CT841 (CPN0998) | 15 | FPTAFFFLL | 167 |
| CT841 (CPN0998) | 566 | ILMAATNRP | 236 |
| CT841 (CPN0998) | 66 | KTALNDNLV | 244 |
| CT841 (CPN0998) | 638 | LLNEAALLA | 254 |
| CT841 (CPN0998) | 735 | ELYDQLAVL | 256 |
| CT841 (CPN0998) | 318 | ALEKQDPEV | 264 |
| CT841 (CPN0998) | 453 | SLGGRIPKG | 303 |
| CT841 (CPN0998) | 380 | FMPI1LVLL | 314 |
| CT841 (CPN0998) | 644 | LLAARKDRT | 315 |
| CT841 (CPN0998) | 423 | VTFADVAGI | 427 |
| CT841 (CPN0998) | 142 | YTISPRTDV | 467 |
| CT841 (CPN0998) | 464 | LIGAPGTGK | 495 |

Predicted Epitopes from C. trachomatis Homologs to Identified C. pneumoniae Proteins

The peptide epitope may be part of a fusion protein or coupled to a carrier moiety.

A frequently used method to predict peptide binding to MHC involves motif searches. The most elaborate motif search uses entire matrices representing the extended motif of the MHC. Although the sequence independent combinatorial specificity may be correct as an average consideration, it is certainly known to be wrong for individual peptides. Furthermore, crystal structures have demonstrated that the interactions at one sub-site may affect interactions at other sub-sites.

Artificial neural networks (ANN) are particularly well suited to handle and recognize any such non-linear sequence information. Information can be trained and distributed into a computer network with input layers, hidden layers and output layer all connected in a certain structure through weighted connections. Such ANN can be trained to recognize inputs (peptides) associated with a given output (say MHC binding). Once trained, the network should recognize the complicated peptide patterns compatible with binding. Using the ANN approach, the size and quality of the training set becomes of major importance. This is particularly true for the HLA since only about 1% of a random set of peptides will bind to any given HLA.

Thus, to generate as few as 100 examples of peptide binders would, if random peptides were screened, require the synthesis and testing of about 10,000 peptides. This would be a very resource demanding and laborious proposition even at this modest number of binders in the training set—and this has to be repeated for every HLA to be tested.

Accordingly, in connection with the present invention matrix predictions were used to scan the SWISS-PROT (http:/lwww.expasy.ch/sprot/) database for potentially high affinity binding epitopes. A large number of these have been synthesized and tested in biochemical binding assays. As predicted, a much higher representation of high affinity binders was obtained (about 80%). These data were subsequently used to train ANN.

For four out of four MHC class I molecules examined, the ANN performed better than the matrix-driven prediction. The predictions have been generated in a fashion, which predicts the actual binding IC50 value rather than an arbitrary classification into “binders” vs. “non-binders”. Indeed, it has been possible to predict binders over a large range leading to the identification of high affinity binders as well as binders of lower affinity as well as non-binders.

The invention further comprises the use of a peptide epitope of the invention for the preparation of a vaccine, as well as a vaccine comprising a peptide epitope of the invention, which vaccine optionally contains acceptable excipients.

In yet another aspect of the invention it relates to the use of a protein of the invention, an antibody of the invention, a nucleic acid compound of the invention or a peptide epitope of the invention in the preparation of a pharmaceutical composition for treating or preventing infection due to an intracellular bacteria, such as a Chlamydia infection, or, alternatively, in the preparation of a diagnostic reagent for detecting the presence of an intracellular bacteria, such as Chlamydia, or antibodies raised against the intracellular bacteria.

The invention further provides a method of inducing an immune response in a human, which comprises administering to said human an immunological effective amount of a protein, an antibody, a nucleic acid compound or a peptide epitope of the invention, and especially such methods for treating or preventing infection of humans by an intracellular bacteria, such as C. pneumoniae or C. trachomatis.

Finally, the invention provides methods of producing a protein or a fragment thereof of the invention or a peptide epitope of the invention, respectively, which comprises transforming, transfecting of infecting a host cell with a vector comprising a nucleic acid compound that encodes said protein or peptide epitope, and culturing the host cell under conditions, which permit the expression of said protein or fragment by the host cell.

The invention is further illustrated by the following, non-limiting examples.

### EXAMPLES

### Example 1

Infection of Mammalian Cell Cultures

Semi-confluent HeLa, HEp-2 or McCoy (ATCC, Rockville, Md., USA) cell monolayers were infected with one inclusion forming unit (IFU) of C. pneumoniae VR1310, C. trachomatis serovar A (HAR-13), D (UW-3/Cx) or L2.(434/Bu)(ATCC) as previously described in [19.] and [17.] The infection medium consisted of RPMI 1640, 25 mM HEPES, 10% FCS. 1% w/v glutamine, 10 mg/ml gentamycin for C. trachomatis A and D and RPMI 1640, 25 mM HEPES, 5% FCS. 1% w/v glutamine, 10 mg/ml gentamycin for C. trachomatis L2.

### Example 2

Pulse Labelling/Chase

To label chlamydial proteins for two hour periods, infected cell cultures were incubated in a medium containing RPMI 1640, 10 mg/ml gentamycin, 40 μg/ml cycloheximide, 100 μCi/ml [35S]-methionine/cysteine (Promix, Amersham Pharmacia Biotech, Uppsala, Sweden) as described previously (Shaw et al., 1999,2000) [18.] [19.]. After labelling the labelling medium was changed to normal growth medium following two washes in normal growth medium and the infected cells were harvested at different points in time after labelling. Similarly, labelled EB proteins were obtained by allowing the Chlamydia to grow until 72 h.p.i. after the two hour labelling periods. The labelled EB were then harvested and purified using two consecutive steps of density gradient ultracentrifugation essentially as described for C. trachomatis in (Schacter and Wyrick, 1994) [22.]and for C. pneumoniae (Knudsen et al. 1999 [17.]). Proteins in the EB preparation and pulse chase preparation were labelled at the same intervals as proteins from the whole cell lysate preparation to facilitate correct 2D-PAGE protein profile comparison.

### Example 3

Sample Preparation

Following [35S]-labelling cells were washed twice in PBS and solubilised in a standard lysis buffer containing 9 M Urea, 4% w/v 3-[(3-cholamidopropyl)dimethylammonium]-1-propanesulfonate (CHAPS; Roche, Germany), 40 mM Tris Base, 65 mM DTE and Pharmalyte 3-10 (Amersham Pharmacia Biotech). For the enrichment of high molecular weight and hydrophobic proteins 7 M urea, 2 M thiourea, 4% w/v 3-[(3-cholamidopropyl)dimethylammonium]-1-propanesulfonate (CHAPS; Boehringer Mannheim, Germany), 40 mM Tris Base, 65 mM dithioerythretiol (DTE) and 2% vol/vol Pharmalyte 3-10 (Amersham Pharmacia Biotech) was used essentially according to (Harder et al. 1999 [23.]). Samples containing whole cell lysates or purified EB were sonicated and centrifuged at 10 000×g for 10 min. Samples were stored at −70° until used.

### Example 4

Separation of Chlamydia Proteins

Chlamydia proteins from whole cell lysates and purified EB were separated by two-dimensional gel electrophoresis essentially as described in (Shaw et al., 1999,2000) [18.] ([19.].

For isoelectric focusing in the first dimension, 18 cm long pH 3-10 NL (non-linear), 4-7 L (linear) or 6-11 (liniar) immobilized pH-gradient drystrips (Amersham Pharmacia Biotech) were reswelled with a sample amount of 200.000 counts per minute (cpm) labelled protein in 350 μl of lysis buffer for 12 hours at 20° C. using the IPGphor™. Other strips used in the invention include ultranarrow IPG strips. The strips are described in Table IX. These allows us to focus on specific pH intervals containing proteins of interest, if necessary. The voltage during isoelectric focusing at 20° C. was programmed as follows: 1 h at 300 V, 2 hours at 300-500 V (linear increase), 1 h at 1000 V, 1 h at 2000 V, 3 h at 3500 V and 5000 V at 24 h when using 3-10 NL, 4-7 Land 6-11 L drystrips.

TABLE IX

| | | Covered pH | |
|---|---|---|---|
| IPG strip name | Liniarity | interval | Strip length |
| Immobiline Drystrip pH 3-10 | non-liniar | 3-10 | 18 cm |
| Immobiline Drystrip pH 3-10 | liniar | 3-10 | 18 cm |
| Immobiline Drystrip pH 4-7 | liniar | 4-7 | 18 cm |
| Immobiline Drystrip pH 6-11 | liniar | 6-11 | 18 cm |
| Immobiline Drystrip pH 6-9 | liniar | 6-9 | 18 cm |
| Immobiline Drystrip pH 3.5-4.5 | liniar | 3.5-4.5 | 18 cm |
| Immobiline Drystrip pH 4-5 | liniar | 4-5 | 18 cm |
| Immobiline Drystrip pH 4.5-5.5 | liniar | 4.5-5.5 | 18 cm |
| Immobiline Drystrip pH 5-6 | liniar | 5-6 | 18 cm |
| Immobiline Drystrip pH 5.5-6.7 | liniar | 5.5-6.7 | 18 cm |

List of examples of commercial available IPG drystrips usefull in the invention

After the first dimension the drystrips were equilibrated in a buffer containing 6 M Urea, 30% v/v glycerol, 2% w/v DTE, 2% w/v SDS, 0.05 M Tris-HCl pH 6.8 for 15 min. The strips were then equilibrated for additionally 15 min. in a buffer in which DTE was replaced by 2.5% w/v iodacetamide. For the second dimension, the Protean II xi Multicell system (Bio-Rad, Richmond, Calif., USA) was used to separate proteins on 9-16% linear gradient SDS-PAGE gels (18 cm×20 cm×1 mm). Analytical gels were fixed in a solution containing 10% acetic acid and 25% 2-Propanol for 30 min. and treated with Amplify (Amersham Pharmacia Biotech) for 30 min. Labelled proteins were visualized by autoradiography after 8-10 days exposure of Kodak Biomax-MR film (Amersham Pharmacia Biotech) at −70° C.

FIG. 1A shows an example of a autoradiography of a high resolution analytical 3-10 IPG 2D-gel (standard lysis buffer was used) of C. trachomatis D proteins labelled from 22-24 h.p.i. FIG. 2A shows an example on a autoradiography of a 3-10 IPG 2D-gel (thiourea containing lysis buffer was used) of C. pneumoniae proteins labelled from 55-57 h.p.i. A total of approximately 600 protein spots could be visualized on each gel as estimated by means of the Melanie II software.

To prepare samples for analysis by mass spectrometry, 2D gels were run with 500-1000 μg of whole cell lysates. To visualize proteins on preparative gels on X-ray films 2×10⁶ cpm of [35S]-protein labelled protein from cells grown in parallel with the unlabelled samples was run on the same gels. Preparative gels were washed for 10 min. in ddH2O and dried un-fixed. Radioactive ink was used to mark anchor spots on the sides of the gels, so that an exact matching of the dried gel and the corresponding X-ray-film could be performed after exposure. Proteins of interest were excised and pooled together from minimum three identical gels. Gels using narrow or ultra-narrow drystrips (Table IX) were used to increase the separation distance, if host cell contamination was a problem in the mass spectrometric identification.

### Example 5

Identification of Vaccine Candidates Using MALDI MS, ESI-Q-TOF MS, PSD MALDI MS and Edman Degradation

Protein spots from preparative gels of whole cell lysates representing vaccine candidates were subjected to in-gel digestion with trypsin. The resulting peptides were purified using reverse phased columns (Gobom et al, 1999 [20.]) or beads (Gevaert et al., 1997 [21.]) consisting of Poros R2 material. The samples were subsequently analyzed using a Bruker REFLEX MALDI time of flight mass spectrometer (Bruker-Daltonik, GmbH, Bremen, Germany) operating in reflectron mode. The resulting masses were compared to peptide masses generated by a theoretical tryptic cleavage of proteins present in databases by peptide mapping as described previously (Schevchenko 1996 [24.]).

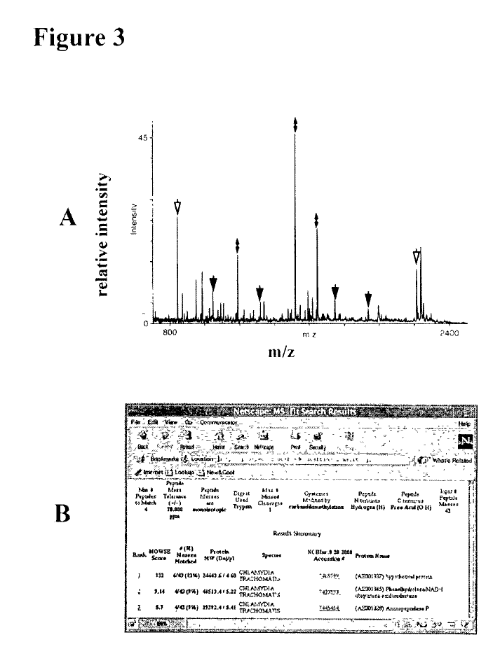

An example of an identification of DT1 as CT668 using MALDI-MS is shown in FIG. 3. FIG. 3A shows the peptide mass fingerprint obtained by an MALDI mass spectrometer. Obtained masses were matched to theoretical tryptic cleavage products of all proteins present in databases using the Prospector software MS-Fit. If the search was restricted to a pI/Mw area in proximity to the protein found on the gels the highest-ranking protein was CT668 (FIG. 3B). However, as proteins from the host cell are sometimes present in the spots from gels with whole cell lysates identification may be unambiguous. Therefore, tandem mass spectrometry and post source decay (PSD) analysis was used to verify the results, if necessary (Reviewed in Mann and Wilm, 1995 [25.], Gevaert, 1997 [21.]).

Tandem mass spectrometry of peptides generated by in-gel digestion was performed on an Electrospray Ionization Quadrupole Time-Of-Flight (ESI-Q-TOF) mass spectrometer (Micromass, Manchester, UK). Using this method a single ionized peptide can be isolated from the sample. Through fragmentation of this parent ion by collision with a gaseous atmosphere several new ions were generated and recorded in a new peptide mass fingerprint. These new ions were distinguished in size by only one amino acid, thus providing details of the amino acid sequence of the original peptide (Mann and Wilm, 1995) [25.].

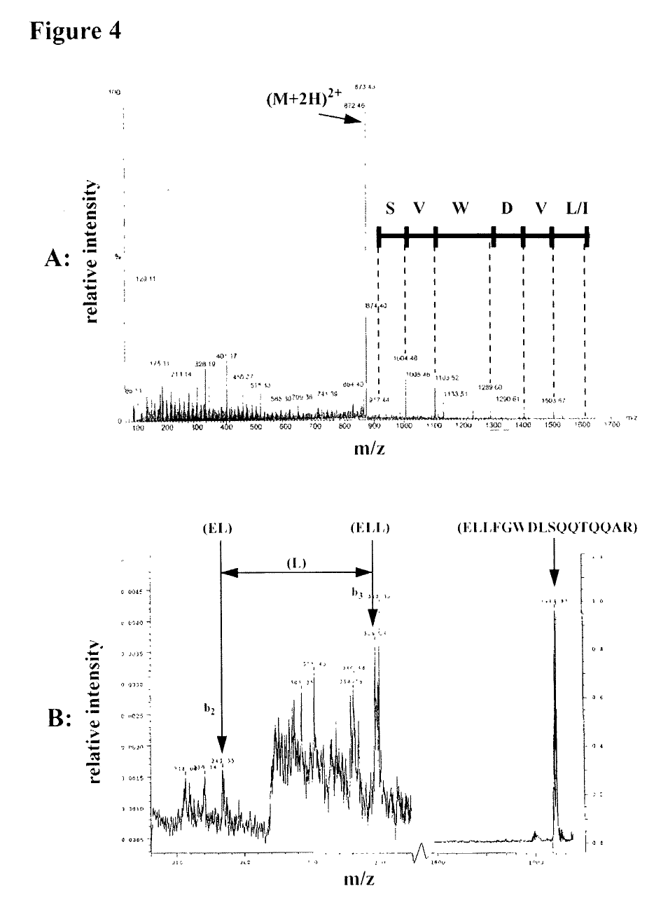

FIG. 4A shows an example of a fragmented peptide parent ion from DT1, yielding a sequence, which through database search using BLAST or MS-Tag were found to correspond to fragments of CT668. Sequence tags arising from the human progesterone binding protein were also identified from the CT668 sample.

Peptides from this protein could also be detected in the MALDI MS peptide mass fingerprint (double headed arrows, FIG. 3A) if the search was restricted to human proteins. This shows that problematic spots, which contain more than one protein, can be unambiguously identified by ESI-Q-TOF, thus conforming problematic MALDI MS identification.

Another approach used in the invention to confirm MALDI results was PSD. PSD utilizes that peptides undergo metastable decay after ionization meaning that peptide fragments of the same velocity have different mass and therefore possess different kinetic energy. The differences in kinetic energy can be resolved by reflecting the fragments in a magnetic field. High energy fragments will penetrate further into the magnetic field than low energy fragments and thereby be delayed. The spectra resulting from fractionation of a single peptide can be used to deduce the amino acid sequence of a peptide sequence tag (PST)(Mann et al, 1993) [28.] as fragmentation predominantly occurs at the peptide bonds. PSTs can be matched against protein databases and thereby the protein from which they originate can be identified (Wilkins et al, 1996 [27.]).

An example of the identification of spot no. CP63 as CPN1016 through PSD MALDI MS is shown in FIG. 4B. From a total of 36 observed masses in the PSD spectra 16 could be matched within one mass unit to masses originating from a theoretical fragmentation of the peptide with the sequence of the 1919.80 Da parent ion (R)ELLFGWDLSQQTQQAR(L), which matched the CPN1016 protein from C. pneumoniae.

Using mass spectrometric approaches, examples of the identification of C. trachomatis D (Table IIIA) and C. pneumoniae (Table IIIb) vaccine candidates are provided. Mw and Pi values were determined electrophoretically with an average error of +/−10%.

CT858 had a theoretical Mw of 67 kDa, thus indicating that the protein identified was a processed fragment of a larger protein. indeed all three peptides from ESI-Q-TOF analysis of DT4 was located in the C-terminal part of the protein.

Another spot, DT48, located in the basic region of the gel also contained CT858. All the peptides identifying DT48 as CT858 was matching the N-terminal part of the protein suggesting that DT48 represents the N-terminal fragment of CT858.

CT610 was identified from EBs by both MALDI MS and Edman degradation in both C. trachomatis D and L2. However, the protein was significantly reduced in EB compared to whole cell lysates and was therefore still considered a candidate for vaccine. The identification of CT610 by Edman degradation was done from C. trachomatis L2 CT610. The N-terminal was determined to be MNFLDQ, which is different from the MMEVFMNFLDQ sequence predicted from the Chlamydia Genome Project (Stephens et al 1998b)[35.].

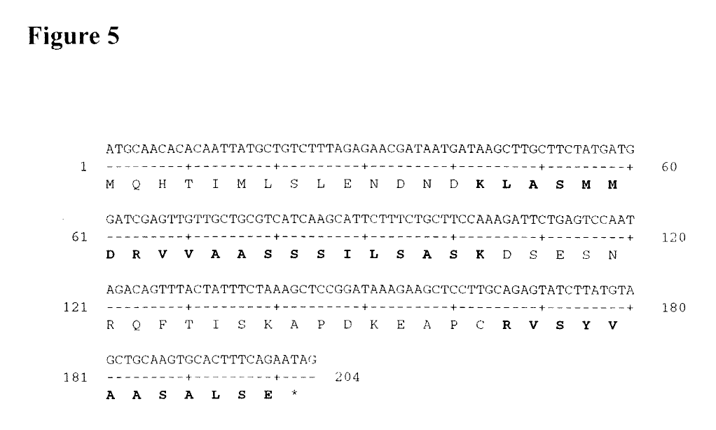

DT8 was identified based on four sequence tags generated by ESI TOF MS. These sequences did not correspond to any predicted open reading frame in the C. trachomatis D genome [35.]. However, by searching the Chlamydia genome in all 6 reading frames with BLAST significant matches could be generated for all four sequence tags. Analysis of the DNA sequence encoding the peptides and their surroundings elucidated a novel open reading frame including a ribosomal binding site, which comprised a 7.2 kDa protein. The translated DNA sequence of DT8 is shown in FIG. 5. This finding illustrates how mass spectrometric approaches used in this invention can identify potentially important ORFs encoding vaccine candidates, which may be neglected in large genome sequencing projects.

Spot CP63 from C. pneumoniae was identified as an N-terminal fragment of CPN1016 the C. pneumoniae homologue of CT858 (FIGS. 2B and C, Table IIIB) indicating processing of these proteins in both Chlamydia species.

In the following examples more detailed investigations of the properties of the examples on identified proteins will be presented.

### Example 6

Comparison of Whole Lysates of Infected Cells to Purified RB

C. trachomatis or C. pneumoniae infected cells were labelled with [35S]-methionine/cysteine for a two-hour period in the presence of cycloheximide as described in Example 2. At the end of the labelling period the infected cells were either harvested directly in lysis buffer as described in Example 3 or used for immediate purification of chlamydia RB. The purification of RB was performed by density gradient ultracentrifugation essentially as described by Schachter and Wyrick, 1994 [22.].

In FIG. 10, examples of regions from gel images of C. trachomatis D proteins from whole lysates of infected HeLa cells labelled from 22-24 h.p.i. are compared to corresponding regions from gel images of RB and EB purified from C. trachomatis D infected HeLa cells labelled from 22-24 h.p.i. Identification by mass spectrometry was obtained for DT4 (C-terminal fragment of CT858), DT48 (N-terminal fragment of CT858), DT23 (Mip), DT76 (hypothetical protein CT691) and DT77 (hypothetical protein CT263) as listed in Table III.

In FIG. 11, examples of regions from gel images of C. pneumoniae infected HEp-2 cells labelled at 55-57 h.p.i. and from purified EB labelled at points in time throughout the developmental cycle are compared to corresponding regions from images of C. pneumoniae infected cell cultures labelled at 34-36 hpi and either harvested as whole lysates of infected cells at 36 h.p.i. or purified as RB at 36 h.p.i.

The protein spots encircled in FIG. 11 were identified as follows. FIGS. 11A and E show CP34 and CP63, which have been identified as two fragments of CPN1016. FIG. 1B shows CP37, which has been identified as CPN0998. FIG. 11C shows CP46 and CP47, which have been identified as CPN0796 and CPN0705, respectively. FIG. 11D shows CP52 which has been identified as CPN0152. FIG. 11F shows CP75 which has been identified as CPN0619.

### Example 7

Detection and Identification of Proteins Located in Type III Secretion Gene Subclusters

Whereas the type III secretion genes in most intracellular bacteria are located in one gene cluster as an pathogenesis island, the Chlamydia type III secretion genes have been identified in three different subclusters located different places in the genome in both C. trachomatis and C. pneumoniae (Stephens et al., 1998 [4.]and Kalman et al. 1999 [5.]). As part of a global proteomic analysis of C. trachomatis A,D and L2 and C. pneumoniae VR1310, proteins which were present in the Type III secretion clusters were identified from gels run with purified EB.

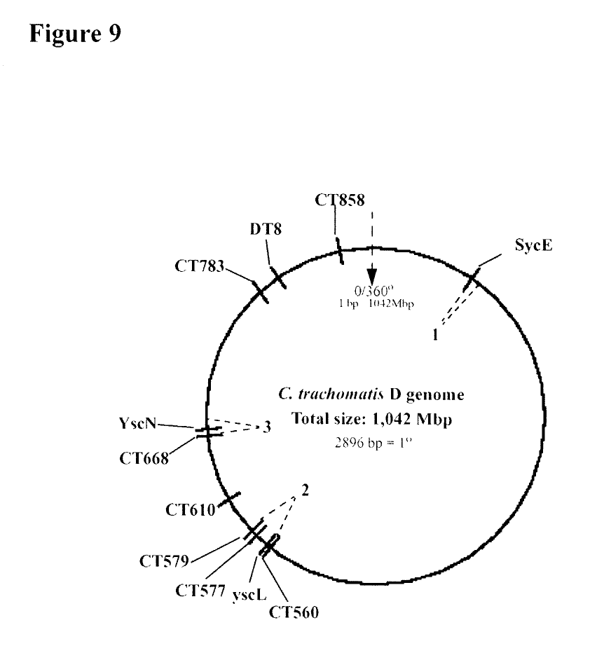

Identified type III secretion proteins from C. trachomatis include the Yop secretion ATPase (yscN), the Yop translocator proteins L (YscL) and the secretion chaperone (SycE) necessary for the transport of proteins from the bacterial cytoplasm to the secretion machinery. Additionally identified C. trachomatis D proteins, which have unknown functions but are located in type III secretion subclusters, include CT560 and the abundant CT577 and CT579. CT668, is clearly present in whole cell lysates, but absent from purified EB, and due to its localization next to YscN, this protein may be secreted. The genomic location of the proteins is shown in FIG. 9.

For C. pneumoniae the type III secretion apparatus proteins LcrE (CPN0324), YscC (CPN0702), YscN (CPN0707) and YscL (CPN0826) have been identified. YscC (CP89 Figure II, Table II) is absent from purified EB, probably due to localization in the inclusion membrane, where it is exposed to the host cell cytoplasm. Two proteins present in a type III cluster located around YscC (CPN0702) and YscN (CPN0707) were detected in considerably higher amount in whole cell lysates than in purified EB. These were CPN0705 (CP90, Figure II, Table II) and CPN0711 (CP76, Figure II, Table II). These proteins may also be located in the inclusion membrane or present in the host cell cytoplasm and in both cases these proteins may be accessible for the proteasome.

### Example 8

Pulse Chase Studies of Candidates for Secreted Proteins

In order to estimate the time in which the identified candidate proteins could be present inside infected cells, the invention provides a series of pulse chase studies. In the following examples infected cell cultures were [35S]-labelled from 22-24 hours. After the labelling period the medium was changed to normal RPMI growth medium without [35S]-methionine and cells were harvested at different times after labelling. Results from several independent studies showed minor variation, probably due to small differences in the host cell density and/or the efficiency of infection.

FIGS. 1B,C,D and E provides an example of a pulse/chase experiment. The intensities of CT668 and DT8 (FIG. 1: A, B, respectively) were significantly decreased 1.5 hours after synthesis and virtually absent from the gels at 4.5 hours and until the EB stage. CT610 and CT783 (FIG. 1: C, D) decreased significantly, but was still detectable at 4.5 hours and until the EB stage. The C-terminal fragment of CT858 (D) (and the N-terminal fragment of CT858) was gradually increased during the first chase periods, but absent from EB, suggesting that the cleavage product accumulated during the Chlamydial development. The N-terminal fragment of the C. pneumoniae homologue of CT858, CPN1016 was also absent from EB (FIG. 2)

### Example 9

Pulse Chase Studies in Combination With Proteasome Inhibitors

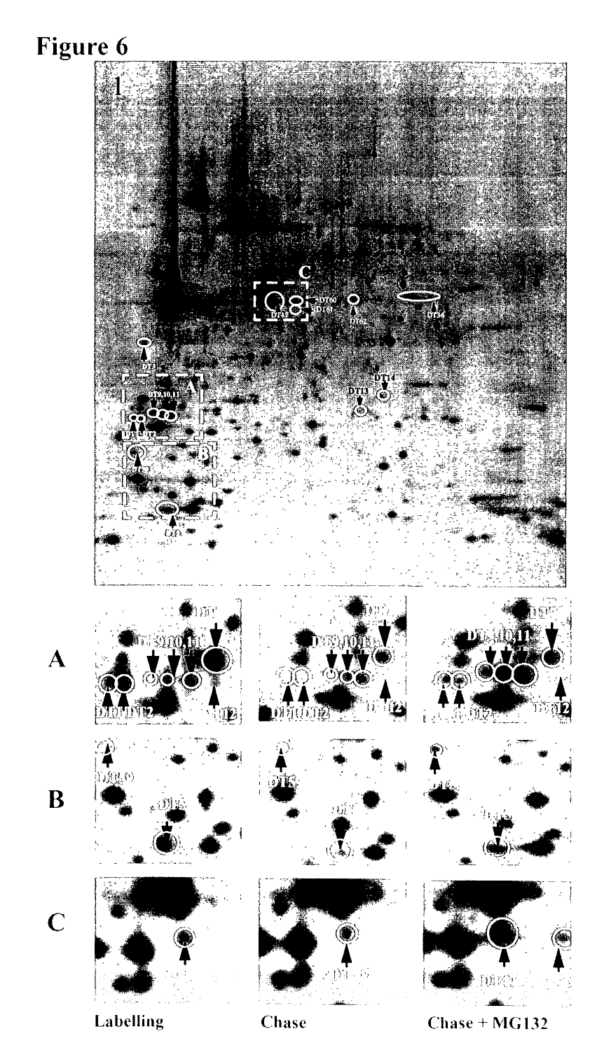

This example shows how to determine which of the vaccine candidates that are processed in the proteasome. Cell permeable proteasome inhibitors are added to the infected cell cultures during the labelling and chase period and the turnover time of proteins compared to that observed for labelling/chase without proteasome inhibitors added. An example of the importance of this approach is shown in FIG. 6. Proteins were labelled in the presence or absence of 10-100 μM of the proteasome inhibitor MG-132 from 22-24 h.p.i. The labelling medium was then replaced with growth medium with or without MG132 after two washes in normal growth medium and chased from 24 h.p.i. to 28 h.p.i. Cell lysates were run on 2D-PAGE(IPG) and compared to controls without MG-132. and to gels with proteins harvested immediately after labelling (FIGS. 6A, B and C). The invention provides examples of fifteen C. trachomatis D proteins, which had an increased turnover time due to treatment with proteasome inhibitors.

The levels of DT9, DT10 and DT11 were actually higher in chased+MG132 gels than in controls harvested immediately after the labelling period. This indicates, that DT9, DT10 and DT11 are very rapidly degraded by the proteasome. In contrast the levels of DT7 were not significantly affected by the addition of proteasome inhibitors.

The invention includes the use of several other proteasome inhibitors (e.g. MG115, MG262, PSI and lactocystein), which inhibit different parts of the catalytic activity of the proteasome, which may elucidate other proteins that are degraded in the proteasome (Table IV).

### Example 10

Using Genetically Altered Cell Lines to Assay the Effect of Proteasome Inhibitor on the Turnover Time of Chlamydia Proteins.

The invention also provides the use of the commercially available mouse embryonal cell lines MEC-PA28 cell line PW8875. MEC-PA28 is transfected with the IFN-γ inducible PA28 alpha and beta subunit of the proteaosome and MEC217 cell line is transfected with IFN-γ inducible LMP2, LMP7 and MECL of the proteasome. MEC-PA28 and MEC217 is grown to semi confluence and infected with C. trachomatis or C. pneumoniae. Control mouse embryonal cell lines, which is not transfected with the proteoasome subunits, were infected in parallel.

As the transfected genes encoding the overexpressed subunits from the cell lines MEC-PA28 and MEC217 are essential for the processing and presentation of MHC class I antigens the experimental procedures using proteasome inhibitors combined with pulse labelling/chase is performed with these host cell as mentioned previously.

### Example 11

Cloning and Expression of Open Reading Frames (ORFs) Encoding Vaccine Candidates

Cloning and expression of the ORFs encoding vaccine candidates was done using the pET-30 LIC Vector Kit (Novagen, Madison, USA) in accordance with the instructions of the manufacturer.

The primers used for PCR of the genes had the following 5′ end and 3′-end LIC overhangs:

Forward primer:
5′ GACGACGACAAGATX-gene specific sequence 3′
Reverse primer:
5′ GAGGAGAAGCCCGGT-gene specific sequence 3′.

Either full-length genes or genes without the leader sequence were amplified by PCR using the Expand™ High Fidelity PCR (Roche, Germany). Thirty-five PCR-cycles were performed on a DNA Thermal Cycler (GeneAmp PCR system 9600, Perkin Elmer) as follows: 15 s at 92° C. (denaturation), 15 s at 55° C. (primer annealing) and 4 min. at 68° C. (extension). The resulting PCR products included the LIC-overhang proximal to the gene specific sequence. The PCR products were Wizard (Promega, Madison, USA) purified and ligated into the pET-30 vector. The pET-30 vector contains the gene encoding kanamycine resistance and a Histidine tag upstream of the LIC cloning site.

pET-30 vectors containing the candidate genes were transformed into competent E. coli Nova Blue strain. Colonies were selected on kanamycin agar plates, and control PCR on selected colonies was performed using a vector specific primer and an insert specific primer. Plasmid DNA was purified from positive colonies containing the candidate gene specific insert. The plasmid DNA was subsequently transformed into E. coli(BL21). Insert positive colonies were selected on kanamycin agar plates. Expression of the fusion protein comprising the gene specific insert including an N-terminal located histidine tag, was induced in 500 ml LB-medium by addition of 1 mM IPTG (Apollo Scientific, GB).

E. coli (BL21) expressing recombinant fusion proteins of CT668,CT858,CT783, CT610, YscN and DT8 from C. trachomatis D and CPN0116 and YscC from C. pneumoniae VR1310 were generated.

The recombinant fusion proteins were purified from lysed bacteria using a nickel resin column (High Trap Sepharose, Amersham Pharmacia Biotech) as previously described. Coomassie stained SDS-PAGE gels of the purified proteins were run. Coomassie stained bands representing fusion proteins was unambiguously identified by MALDI MS to verify that the correct fusion protein had been generated. Sera containing polyclonal antibodies were obtained by immunizing New Zealand Whiterabbits intramuscularly three times with 50 μg of fusion protein dissolved in Freunds adjuvant and intravenously twice with 50 μg of fusion protein dissolved in PBS as described in [17] by Knudsen et al.

### Example 12

Western Blotting Using PAbs Against Vaccine Candidates

In order to confirm that the PAbs recognized the correct vaccine candidates and to visualize potential post translational modifications or processing, 2D-PAGE immunoblotting were performed. 500 μg unlabelled and 2×10⁶ cpm labelled C. trachomatis D or C. pneumoniae protein from whole cell lysates was separated by 2D-PAGE, and proteins were electroblotted on to PVDF membranes. Immunostaining was carried out using a 1/5001/1000 dilution of the PAbs in a buffer containing 150 mM NaCl (or in high salt, 400 mM), 20 mM Tris, 0.2% w/v gelatin, 0.05% v/v Tween 20 (Bio-Rad) and 2% v/v normal goat serum (Dako, Glostrup Denmark).

The secondary antibody used was alkaline phosphatase-conjugated goat anti-rabbit IgG (Bio-Rad) diluted 1/2000 in antibody buffer. Blots were stained with 5-bromo-4-chloro-3-indolylphosphate toluidium (BCIP)/nitroblue tetrazolium (NBT) (Bio-Rad) until a clear reaction was detected. Immunoblots were computer scanned and subsequently exposed to X-ray films for approximately 8 days. Melanie II Software was used in order to compare immunostained proteins with their [35S]-labelled counterparts.

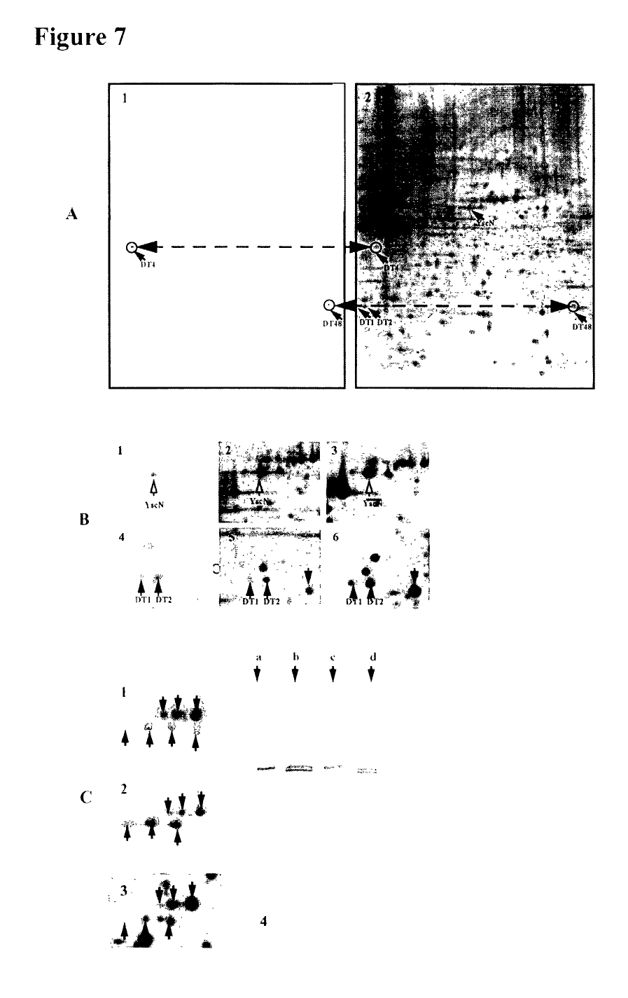

In FIGS. 7A1 and 7A2 an example of a total 2D-PAGE IMB image with PAb245 against CT858 is shown. PAb 245 reacts reproducible with DT4 (CT858 C-terminal fragment) and DT48 (CT858 N-terminal fragment) (FIG. 7A1) as indicated from the corresponding labelled background (FIG. 7A2).

FIGS. 7B1 and 7B4 shows IMB with CT668 and YscN. Immunoreactive protein spots on the gel and their localization based on a labelled background (FIGS. 7B2 and B5) corresponded to the positions of the proteins on analytical gels (FIGS. 7B3 and B6), thus confirming that the PAbs reacted with the correct proteins.

The invention provides an example of IMB showing that levels of certain isoforms of CT610 are clearly increased in MG132 treated infected cells compared to non-treated controls. DT7 was identified as CT610 and was located just above DT9, DT10, DT11 and DT12 (FIG. 6A). IMB with PAb255 against CT610 on 2D-gel blots (FIG. 7C1) of whole cell lysates clearly reacted with two rows of spots, one row representing DT7 and the other row representing DT9, DT10, DT11 and DT12. CT610 is therefore present in different isoforms presumably due to different posttranslational modification and processing. The abundance of DT9, DT10 and DT11 was clearly increased if cells were treated with the proteasome inhibitor MG132 (FIG. 7C2).

IMB with PAb255 was performed on ordinary SDS-PAGE PVDF blots with protein from whole cell lysates treated for six hours in the presence or absence of MG132, respectively. FIG. 7C4, clearly shows an extra band below the band representing the DT7 row of spots in lanes containing proteins from MG132 treated infected cells (lane b and d) compared to untreated controls (lane a and c). The levels of the upper band, representing the DT7 row, was not significantly altered by the proteasome inhibitor treatment.

### Example 13

Expression of Candidates in Different C. trachomatis Serovars and Upon Growth in Different Host Cells

The invention considers serovar/host cell specific differences in expression levels of potentially secreted proteins, as host cell/Chlamydia interactions may be different for other serovars or by cultivation in other host cells. This is relevant for the choice of the vaccine candidate with greatest potential for a general C. trachomatis vaccine.

CT668, CT858, CT783 and CT610 were all detected at the same positions in C. trachomatis A, D and L2. This is in agreement with the very high conservation of the genes encoding these proteins between C. trachomatis D and L2. In addition, all of the proteins were expressed when C. trachomatis A, D and L2 was cultivated in McCoy or Hep-2 cells instead of HeLa cells, indicating that the expression of these proteins is independent of cell types used. However, based on gels with Chlamydia protein labelled from 22-24 h.p.i. or 34-36 h.p.i. DT8 was detectable at pI 5.1 and Mw 7.5 in serovars A and D. However, in serovar L2 DT8 was detected at pI 6.4 Mw 7.5 presumably due to minor amino acid substitutions which alter the net charge and isoelectric point of the protein.

### Example 14

Indirect Immunofluorescence Microscopy of Vaccine Candidates

Wells with glass cover slips containing semiconfluent HeLa cell monolayers were infected with C. pneumoniae or C. trachomatis D. A low titer of Chlamydia was used so that only approximately 50% of the cells was infected, thus making it possible to clearly discriminate between infected and un-infected cells. Cells were washed twice in PBS and fixed in formaldehyde or methanol at different h.p.i. and subjected to indirect immunofluorescence microscopy. If necessary PAbs were pre-absorbed by acetone precipitated HeLa protein in order to obtain minimum cross reaction to human proteins.

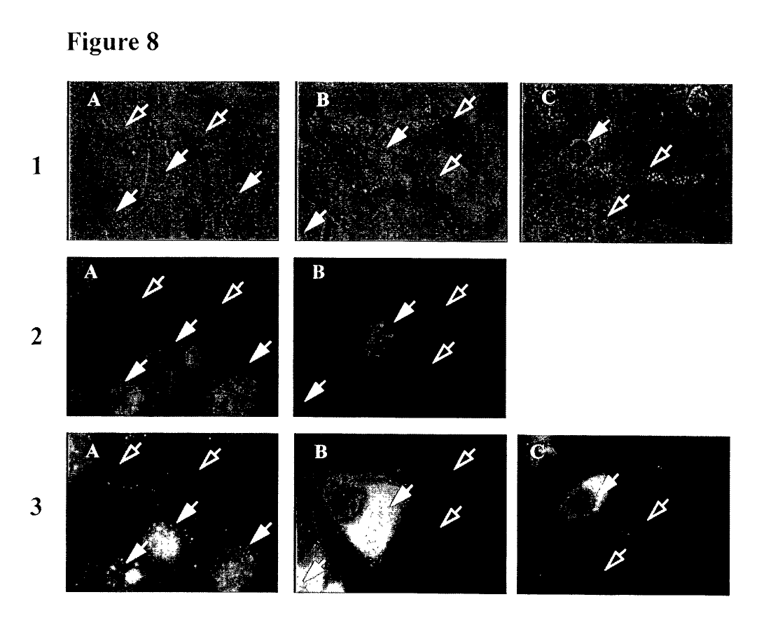

In the example shown in FIG. 8, a 1/200 dilution of pre-absorbed rabbit PAb and a 1/25dilution of mouse monoclonal antibody directed against C. trachomatis MOMP(MAb 32.3) or MAb18.3 against C. pneumoniae was is used. Fluorescein isothiocyanate-conjugated (FITC) goat anti-rabbit (GAR) IgG antibody and rhodamine conjugated goat anti-mouse (GAM) IgG antibody (Jackson, Trichem, Denmark) was used as secondary antibodies. The double immunostaining were performed in order to determine the sub-cellular localization of the vaccine candidates relative to the Chlamydial inclusion.

PAb 249 directed against DT8, which had a short turnover time, reacted weakly with RB in the Chlamydial inclusion (FIG. 8A3). No significant reaction to host cell structures were detected beyond the inclusion despite minimal cross reaction with HeLa cell proteins.

In contrast, CT858 clearly stained the host cell cytoplasm in infected but not un-infected cells (FIG. 8B3). In general, the staining was more intense at the borders of the inclusions. The staining of the host cell cytoplasm could be visualized from 12 h.p.i to 72 h.p.i. in agreement with the long turnover time predicted by the pulse chase studies. The same characteristics of reaction were observed when the PAb raised against C. pneumoniae CPN1016 which is homolog to CT858 was reacted with C. pneumoniae infected Hep-2 cells.

Description of Examples on Identified Proteins

### Example 15

Spot Number CT668 (DT1 and DT2)

CT668 was placed immediately upstream of the YscN ATPase in one of the subclusters containing genes with homology to Type III secretion genes and did not contain a predicted recognizable signal peptidase cleavage site. CT668 was only present in C. trachomatis D for approximately 4-6 hours during which it steadily decreased in abundance. PAbs against CT668 only seems to react weakly with the RB in the inclusion in IMF, but considering the short turnover time of this protein, this may be explained by a fast degradation in the host cell. CT668 has been detected from 12-40 h.p.i., which suggests that the protein is produced during most of the intracellular development. It may therefore be that C. trachomatis is exporting CT668 continuously to the host cell, where it exerts its action and is rapidly degraded.

Two variants of CT668 were identified. The basic variant was most abundant on gels with C. trachomatis proteins labelled from 22-24 h.p.i and subsequently harvested. Interestingly, the intensity of the acidic variant increased and the basic variant decreased with time in all studies performed. When CT668 was chased in 30 min. intervals the increase of the acidic variant was detectable already at one hour (FIG. 1B).

This finding may suggest that the modification is not due to the running conditions of the gel, which can create modifications such as carbamylation or amidation. Instead the modification seems exclusively to originate through an unknown enzyme, which modifies the protein.

Modification of secreted proteins has been described in C. psitacci, where IncA is phosphorylated by a host cell Ser/Thr kinase after translocation to the inclusion membrane (Rockey et al., 1997) [29.]. The turnover time of CT668 was prolonged upon treatment with proteasome inhibitor, suggesting that at least limited amounts of the CT668 produced may be processed in the proteasome.

### Example 16

Spot Number DT8(DT8)

DT8 is a novel 7.2 kDa protein, which based on homology search is a C. trachomatis specific protein. PSORT analysis indicated no recognizable leader sequence for this protein. The theoretical coordinates pI 5.21/7.2 kDa were in excellent agreement with the experimentally determined. Many of the features recognized for CT668 were observed for DT8, as well. A short turnover time of <6 hours was observed and IMF showed only a weak reaction with RB.

After the stop codon in DT8 a potential stem-loop region can be predicted indicating a Rho-independent transcription termination of the protein.

### Example 17

Spot number DT7, DT 9, DT10, DT11, DT12 (CT610)

CT610 was not located near any genes with homology to genes which are involved in secretion in other organisms. The protein was identified from both whole cell lysates and EB, but by means of the Melanie II Software was estimated to be at least 30 times more abundant in whole cell lysates. The protein was detected in several isoforms represented by two molecular weight polymorphisms as determined by means of the Pab255 raised against CT610. These rows of spots represented DT7 (upper row) and DT9, 10, 11, 12 (lower row). Both these row had a short turnover time in the pulse chase studies. Interestingly, the abundance of DT9, DT10 and DT11 was significantly increased upon treatment with proteasome inhibitor based on pulse chase/MG132 studies. The pulse chase studies were verified by SDS PAGE IMB with Pab255.

Thus, the invention provides evidence that certain isoforms of CT610 are secreted and processed in the proteasome. The different fate of the CT610 isoforms shows the relevance of using Pab raised against vaccine candidates to detect such isoforms.

### Example 18

Spot Number DT3 (CT783)

CT783 has been suggested to be a C. trachomatis protein disulfide bond isomerase. CT783 show homology to thioredoxin disulfide isomerase (CT780), and to a protein disulfide isomerase from Methanobacterium thermoautotrophicum. A 33 amino acid leader sequence can be predicted by PSORT and SignalP, and the theoretical pI and molecular weight of the cleaved protein was in agreement with the one experimentally determined. In addition polyclonal antibodies generated against CT783 stained the correct spot in 2D-PAGE IMB using 4-7 L IPG.

Due to the cleavage of the N-terminal leader sequence, this protein is probably not secreted via the Type I or III systems, but more likely the Type II system. A weak PSORT prediction suggested a sub-cellular localization in the bacterial inner membrane, although most PDI are normally located in the periplasm. CT783 had a very short turnover time and is virtually absent in the Chlamydia after approximately 4-6 hours chase following synthesis.

The turnover time of CT783 was prolonged by proteasome inhibitors suggesting a potential processing in the proteasome.

### Example 19

Spot Number DT4 and DT48 (CT858) and CP63 (CPN1016)

CT858 had a cleavable N-terminal leader sequence and was predicted to be a 67 kDa periplasmic protein, but was identified at two different positions on the gels. The sequence tags, which identified DT4 as CT858 all matched the C-terminal part of CT858. In IMB on 2D-PAGE PVDF membrane with whole cell lysates PAb245 reacts clearly and reproducible with this C-terminal fragment. In addition Pab245 reacted with the protein spot DT48, which also has a long turnover time in the pulse chase studies as seen for DT4.

DT48 was also identified as an N-terminal fragment of CT858 by MALDI MS. The molecular coordinates of DT48 were pI 7.3/Mw 25.8. The N-terminal part of CT858 yields a peptide with the coordinates agreeable with the one determined experimentally using the pI/Mw tool on different lengths of the N-terminal (without the signal peptide). This analysis suggests a cleavage site around K²³³S²³⁴M²³⁵.

The fact that the N-terminal and C-terminal fragment of CT858 can be detected on 2D-gels all the way up to the EB stage (72 h.p.i.), but not in purified EB is indicative of a long turnover time in the host cell cytoplasm. This was in agreement with the very clear detection of CT858 in the host cell cytoplasm up to 72 hours by IMF studies. CT858 showed weak homology to the tail-specific protease (tsp) from E. coli, which has been involved in the processing of penicillin binding proteins and includes a IRBP domain from human interphotoreceptor retinoid-binding proteins, which bind hydrophobic ligands (Silber et al., 1992) [32.]. Type II secretion has been linked to the export of degradative proteins in several gram negative bacteria including P. aeroginosa and Aeromonas hydrophila (Reviewed in Hobbs and Mattick, 1993) [33.] In Aerosiginosa hydrophila a secreted elastase (ahpB) has recently been suggested to be important for the virulence of the organism (Cascon et al., 2000) [34.].

These proteins has similar fates as CT858. They are mostly synthesized as a propeptide with a cleavable signal peptide and then processed to a mature protein. Another C. trachomatis protein, which also shows homology to tail-specific protease, is the hypothetical protein CT441. It remains to be determined whether this protein may show the same secretory characteristics as CT858.

The same sub-cellular localization of CT858 was observed for the C. pneumoniae homolog, CPN1016 with Pab253 against CPN1016, thus indicating a functional conservation of this gene between the two Chlamydia species.

### Example 20

The secreted C. trachomatis D and C. pneumoniae proteins, which were identified by their gene number, were analysed by an ANN trained to recognize peptides with affinity for the human HLA-A2. In Tables V-VIII peptides selected by the ANN for predicted binding to HLA-A2 are listed and the affinity (Kd) is given in nM. The lower the value the better. Most peptides with Kd below 50 nM are immunogenic. Peptides with Kd below 500 nM (but above 50 nM) are potentially immunogenic. The binding of a given peptide may be improved by substitution of a sub optimal amino acid in the anchor positions P2 or P9—a strategy that often will retain the specificity directed against the natural peptide.

### Example 21

Determination of the Ability of a Vaccine Candidate to Generate Specific Cytotoxic CD8+ T-Cells in Experimental Animal Models.

In one approach the vaccine candidates are used as full length recombinant proteins to immunize experimental animals (mouse or guinea pig) including transgenic A2 mice expressing human HLA class I molecules.

In another approach the vaccine candidates are screened for T-cell epitopes by computer algorithms and subsequently peptides encompassing these epitopes are synthesized and used for immunization as described for full length vaccine candidates.

In a third approach 8-10 amino acids long peptides are synthesized, in an overlapping way so that they cover the entire sequence of a vaccine candidate, and used for MHC class I binding assay in competition with radio labelled intermediate binders. Peptide, which are good binders are used for immunization as described for full length vaccine candidates.

The vaccine candidates are administrated either as combinations of peptides/proteins or as single peptides/proteins in adjuvant. The vaccine candidates can also be administrated as a DNA-vaccine or by a virus expressing the vaccine candidate.

Peripheral blood mononuclear cells (PBMC) from the immunized animals are purified by density gradient centrifugation and CD8+ cells are purified by use of antibodies bound to magnetic beads or by other methods.

CD8+ T-cell activity is measured by proliferation assays such as ELISPOT and incorporation of tritiated thymidine and by specific lysis assays (chrome release).

Purified PBMC or CD8+ cells from immunized animals are plated on microtiter plates, in limitting dilution, with irradiated antigen presenting cells, growth factors and a specific or non-specific stimulator. For specific stimulation single vaccine candidate proteins or peptides, which has been predicted as a good T-cell epitopes or found to be a good binders to the MHC class I molecule are used. For non-specific stimulation Chlamydia infected cells are used. The cells are cultured for 9-14 days during which antigen specific cells proliferate. Generation of specific cytotoxic CD8+T-cells is determined by measuring the cytokine secretion from stimulated cells with the ELISPOT assay. The proliferation of T-cells is measured by incorporation of tritiated thymidine followed by scintillation counting. The cytotoxicity of the proliferated cells is measured using a cytotoxic assay as the chromium-release assay using Chlamydia infected cells or recombinant cells expressing the vaccine candidate protein/peptide as target cells [42].

### Example 21

Testing of the Vaccine Candidates for the Ability to Protect Mice and Guinea Pigs Against Chlamydia Infection.

Experimental animals are immunized (as described above) with the vaccine candidates as single proteine/peptides or with a combination of vaccine candidates. Following immunization, the animals are experimentally infected with Chlamydia (intra nasal infection for C. pneumoniae and genital infection for C. trachomatis. Protection against infection is measured by cultivation of the Chlamydia, immunohistochemistry, quantitative PCR and by investigation of seroconversion upon infection.

### Example 23

Determination of the Ability of Chlamydia Infection to Generate Vaccine Candidate-Specific Cytotoxic CD8+ T-Cells in Humans.

Human serum samples are tested by ELISA (Medac) for the presence of antibodies to Chlamydia. Sero-positive individuals are selected for the presence of vaccine candidate-specific cytotoxic CD8+ T-cells.

Peripheral blood mononuclear cells (PBMC) from humans who are tested antibody positive for Chlamydia are purified by density gradient centrifugation and CD8+ cell are purified by use of antibodies and magnetic beads or other method. CD8+ T-cell activity specifically directed against vaccine candidate proteins/peptides is measured by the methods described in example 19.

### Example 24

Using the Vaccine Candidates for the Developments of a ELISA Test for Diagnostic Purposes

As the secreted proteins in the present invention are not present or significantly reduced in the purified microorganisms, immuno assay based on purified elementary bodies cannot detect antibodies to such proteins. Therefore secreted proteins can represent unrecognised major antigens, which are also involved in the humoral immune response. In addition an ELISA based on secreted Chlamydia proteins may detect persistent infection with Chlamydia as the secreted proteins are only expressed during the intracellular stage of Chlamydia development.

1) The secreted proteins are produced as recombinant proteins, which are purified. Alternatively overlapping synthetic peptides representing the secreted proteins are also produced.

2) ELISA plates are coated with purified recombinant proteins representing secreted proteins (or synthetic peptides originating from the secreted proteins). The ELISA plate is blocked with 15% foetal calf serum to avoid unspecific binding

3) Patient sera are screened for antibodies against C. Trachomatis or C. pneumoniae using micro-IF or ELISA (Medac). The positive sera is tested on a ELISA plate coated with the recombinant antigens representing the secreted proteins

4) For detection of antibody binding anti-human IgG, IgA or IgM is used. As positive control sera from infected mice are used.

5) The results from micro-IF or ELISA (Medac) are compared to the ELISA based on recombinant proteins representing the secreted proteins.

### REFERENCES

1. Saikku P, Leinonen M, Mattila K, Ekman M R, Nieminen M S, Makela P H, Huttunen J K, Valtonen V, Serological evidence of an association of a novel Chlamydia, TWAR, with chronic coronary heart disease and acute myocardial infarction. Lancet Oct. 29, 1988;2(8618):983-6

2. Shor A, Kuo C C, Patton D L (1992) Detection of Chlamydia pneumoniae in coronary arterial fatty streaks and atheromatous plaques. S Afr Med J.82(3):158-61

3. Hueck, C. J. (1998) Type III protein secretion systems in bacterial pathogens of animals and plants. Microbiol Mol Rev 62, 379-433

4. Stephens, R. S., Kalman, S., Lammel, C., Fan, J., Marathe, R., Aravind, L., Mitchell, W., Olinger, L., Tatusov, R. L., Zhao, Q., Koonin, E. V., Davis, R. W. (1998) Genome sequence of an obligate intracellular pathogen of humans: Chlamydia trachomatis, Science 282, 754-759

5. Kalman, S., Mitchell, W., Marathe, R., Lammel, C., Fan, J., Hyman, R. W., Olinger, L., Grimwood, J., Davis, R. W., Stephens, R. S (1999). Comparative genomes of Chlamydia pneumoniae and C. trachomatis, Nat. Gent. 21, 385-389

6. Rock, K. L., Alfred L. Goldberg (1999) Degradtion of cell proteins and the generation of MHC class I-presented peptides, Annu. Rev. Immunol, 17, 739-779

7. Murdin A D, Su H, Manning D S, Klein M H, Parnell M J, Caldwell H D (1993) A poliovirus hybrid expressing a neutralization epitope from the major outer membrane protein of Chlamydia trachomatis is highly immunogenic. Infect Immun, 61(10):4406-14

8. Cerrone M C, Ma J J, Stephens R S (1991) Cloning and sequence of the gene for heat shock protein 60 from Chlamydia trachomatis and immunological reactivity of the protein. Infect Immun, 59(1):79-90

9. Igietseme J U, Magee D M, Williams D M, Rank R G (1994) Role for CD8+ T cells in antichlamydial immunity defined by Chlamydia-specific T-lymphocyte clones. Infect Immun 62(11):5195-7

10. Subtil A, Blocker A, Dautry-Varsat A (2000) Type III secretion system in Chlamydia species: identified members and candidates. Microbes Infect 2(4):367-9

11. Fields K A, Hackstadt T (2000) Evidence for the secretion of Chlamydia trachomatis CopN by a type III secretion mechanism Mol Microbiol 2000 December;38(5): 1048-60

12. Read, T. D., Brunham, R. C., Shen, C., Gill, S. R., Heidelberg, J. F., White, O., Hickey, E. K., Peterson, J., Utterback, T., Berry, K., Bass, S., Linher, K., Weidman, J., Khouri, H., Craven, B., Bowman, C., Dodson, R., Gwinn, M., Nelson, W., DeBoy, R., Kolonay, J., McClarty, G., Salzberg, S. L., Eisen, J., Fraser, C. M. (2000) Genome sequences of Chlamydia trachomatis MoPn and Chlamydia pneumoniae AR39. Nucleic Acids Res 28,1397-1406

13. Shirai M, Hirakawa H, Kimoto M, Tabuchi M, Kishi F, Ouchi K, Shiba T, Ishii, K, Hattori M, Kuhara S, Nakazawa T (2000) Comparison of whole genome sequences of Chlamydia pneumoniae J138 from Japan and CWL029 from USA. Nucleic Acids Res 15:2311-2314

14. Starnbach, M. N., Bevan, M. J. (1994), Cells infected with Yersinia present an epitope to class I MHC-restricted CTL., J Immunol, 153,1603-12

15. Rockey, D. D, Heinzen, R. A, and Hackstadt, T. (1995) Cloning and characterisation of a Chlamydia psittaci gene coding for a protein localized in the inclusion membrane of infected cells. Mol. Mcirobiol 15, 617-626

16. Bannantine, J., Stamm, W., Suchland, R., Rockey, D.(1998), Chlamydia trachomatis IncA is localized to the inclusion membrane and is recognized by antisera from infected humans and primates, Inct. immun, 66, 6017-6021

17. Knudsen K, Madsen A S, Mygind P, Christiansen G, Birkelund S (1999) Identification of two novel genes encoding 97- to 99-kilodalton outer membrane proteins of Chlamydia pneumoniae. Infect Immun 67(1):375-83

18. Shaw, A. C., Christiansen, G., Birkelund, S. (1999) Effects of interferon gamma on Chlamydia trachomatis serovar A and L2 protein expression investigated by two-dimensional gel electrophoresis. Electrophoresis 20(4-5):775-80.

19. Shaw, A. C., Christiansen, G., Roepstorff, P., Birkelun, S. (2000) Genetic differences in the Chlamydia trachomatis tryptophan synthase a-subunit can explain variations in serovar pathogenesis. Microbes Infect 2: 581-592

20. Gobom J, Nordhoff E, Mirgorodskaya E, Ekman R, Roepstorff P (1999) Sample purification and preparation technique based on nano-scale reversed-phase columns for the sensitive analysis of complex peptide mixtures by matrix-assisted laser desorption/ionization mass spectrometry. J Mass Spectrom 34(2):105-16

21. Gevaert, K., Demol, H., Puype, M., Broekaert, D., De Boeck, S., Houthaeve, T., Vandekerckhove, J. (1997) Peptides adsorbed on reverse-phase chromatographic beads as targets for femtomolesequencing by post-source decay matrix assisted laser desorption ionization-reflectron time of flight mass spectrometry (MALDI-RETOF-MS). Electrophoresis 18: 2950-2960

22. Schachter, J., Wyrick, P. B (1994) Culture and isolation of Chlamydia trachomatis. Methods Enzymol 236: 377-390

23. Harder, A., Wildgruber, R., Nawrocki, A., Fey, S. J., Larsen, P. M., Gorg, A. (1999) Comparison of yeast cell protein solubilization procedures for two-dimensional electrophoresis. Electrophoresis 20: 826-829

24. Shevchenko A, Jensen O N, Podtelejnikov A V, Sagliocco F, Wilm M, Vorm O, Mortensen P, S, Boucherie H, Mann M (1996) Linking genome and proteome by mass spectrometry: large-scale identification of yeast proteins from two dimensional gels. Proc Natl Acad Sci USA: 93: 14440-14445

25. Mann, M, Wilm, M (1995) Electrospray mass spectrometry for protein characterization. Trends Biochem Sci., 20:219-24

26. Wilm M, Mann M (1996) Analytical properties of the nanoelectrospray ion source Anal Chem 68(1):1-8

27. Wilkins M R, Gasteiger E, Sanchez J C, Appel R D, Hochstrasser D F. Protein identification with sequence tags. Curr Biol. Dec. 1, 1996;6(12):1543-4.

28. Mann M, Hojrup P, Roepstorff P. Use of mass spectrometric molecular weight information to identify proteins in sequence databases. Biol Mass Spectrom. 1993 June;22(6):338-45.

29. Rockey, D. D., Grosenbach, D., Hruby, D. E., Peacock, M. G., Heinzen, R. A., Hackstadt, T.(1997) Chlamydia psittaci IncA is phosphorylated by the host cell and is exposed on the cytoplasmic face of the developing inclusion, Mol. Microbiol., 24, 217-228

30. Forster C, Marienfeld S, Wilhelm R, Kramer R (1998) Organelle purification and selective permeabilisation of the plasma membrane:two different approaches to study vacuoles of the filamentous fungus Ashbya gossypii. EMS Microbiol Lett Oct. 15, 1998;167(2):209-14

31. Melan M A (1999) Overview of cell fixatives and cell membrane permeants. Methods Mol Biol 1999;115:45-55

32. Silber, K. R., Keiler, K. C., Sauer, R. T. (1992) Tsp: a tail-specific protease that selectively degrades proteins with nonpolar C termini. Proc Natl Acad Sci USA, 89, 295-9

33. Hobbs, M, Mattick, J. S. (1993) Common components in the assembly of type 4 fimbraie, DNA transfer systems, filamentous phage and protein secretion apparatus: a general system for the formation of surface-associated protein complexes. Mol. Microbiol. 10, 233-243

34. Cascon, A., Yugueros, J., Temprano, A., Sanchez, M., Hernanz, C., Luengo, J. M., Naharro, G. (2000) A major secreted elastase is essential for pathogenicity of Aeromonas hydrophila. Infect Immun, 68,3233-41

35. Stephens, R. S., Kalman, S., Fenner, C., Davis, R.,(1998b) The Chlamydia Genome Project, http://socrates.berkeley.edu:4231/.

36. Rock, K. L., Gramm, C., Rothstein, L., Clark, K., Stein, R., Dick, L., Hwang, D., Goldberg, A. L. (1994) Inhibitors of the proteasome Block the degradation of Most Cell Proteins and the Generation of Peptides Presented on MHC Class I Molecules. Cell, 78, 761,771

37. Fling, S. P., Sutherland, R. A., Steele, L. N., Hess, B., D'Orizio, S. E. F, Maisonneuve, J.-L., Lampe, M., Probst, P., Starnbach, M. N.(2001) CD8 T cells recognize an inclusion membrane associated protein from the vacuolar pathogen Chlamydia trachomatis, PNAS, 98,1160-1165

38. Sijts, A. J., S. Standera, R. E. Toes, T. Ruppert, N. J. Beekman, P. A. van Veelen, F. A. Ossendorp, C. J. Melief, and P. M. Kloetzel (2000). MHC class I antigen processing of an adenovirus CTL epitope is linked to the levels of immunoproteasomes in infected cells. J. Immunol., 164, 4500

39. Van Hall, T., A. Sijts, M. Camps, R. Offringa, C. Melief, P. M. Kloetzel, and F. Ossendorp (2000) Differential Influence on Cytotoxic T Lymphocyte Epitope Presentation by Controlled Expression of Either Proteasome Immunosubunits or PA28. J. Exp. Med., 192, 483.

40. Shockett, P., M. Difilippantonio, N. Hellman, and D. G. Schatz (1995). A modified tetracycline-regulated system provides autoregulatory, inducible gene expression in cultured cells and transgenic mice. Proc. Natl. Acad.

Sci. USA, 92, 6522

41. Probst, P., Bhatia, A., Skeiky, Y., Jen, S.(1999), International publication number: WO 00/34483, PCT/US99/290120

42. Hickling K. H. (1998), Measuring human T-lymphocyte function, Expert Reviews in Molecular Medicine ISSN 1462-3994

43. Sijts, A. J., Villanueva, M. S., Pamer, E. G: CTL epitope generation is tightly linked to cellular proteolysis of a Listeria monocytogenes antigen, The Journal of immunology, 1996,156,1497-1503

44. Hess, J., Kaufmann, S. H: Vaccination strategies against intracellular microbes, FEMS Immunology and Medical Microbiology, 1993, 7, 95-104

1. A method for identifying proteins secreted from an intracellular bacteria, comprising the following steps:
1) infecting host cells by the intracellular bacteria,
2) labelling the intracellular bacteria present in the infected cells,
3) preparing
a) whole cell lysates of the infected cells
b) purified and lysed bacteria from the infected cells,
4) comparing 2D-gel electrophoresis protein profiles of i) the whole cell lysates from step 3a) with ii) the purified and lysed bacteria from step 3b),
5) detecting protein spots from step 4) which are present in the whole cell lysates but absent or present in significantly reduced amount in the purified bacteria,
6) identifying the proteins in the spots selected in step 5).

2. A method for identifying proteins secreted from an intracellular bacteria, comprising the following steps:
1) infecting host cells by the intracellular bacteria,
2) pulse labelling of the intracellular bacteria present in the infected cells,
3) preparing whole cell lysates of the infected cells after different periods of chase following step 2),
4) comparing 2D-gel electrophoresis protein profiles of the whole cell lysates prepared after different periods of chase from step 3),
5) detecting protein spots from step 4) which are present in decreasing amount as chasing periods increase in step 3),
6) identifying the proteins in the spots selected in step 5).

3. A method for identifying proteins secreted from an intracellular bacteria, comprising the following steps:
1) infecting host cells by the intracellular bacteria,
2) cultivating the host cells in the presence and in the absence of a proteasome inhibitor, respectively,
3) labelling the intracellular bacteria present in the infected cells cultivated in the presence and in the absence of a proteasome inhibitor, respectively,
4) preparing whole cell lysates of the infected cells,
5) comparing 2D-gel electrophoresis protein profiles of the whole cell lysates of the infected cells cultivated in the presence and in the absence of a proteasome inhibitor, respectively,
6) detecting protein spots from step 5) which are present in the is whole cell lysates cultivated in the presence of a proteasome inhibitor, but absent or present in significantly reduced amount in the whole cell lysates cultivated in the absence of a proteasome inhibitor,
7) identifying the proteins in the spots selected in step 6).

4. A method according to any of the claims 1-3, further comprising the following steps:
1) obtaining antibodies against proteins from said intracellular bacteria identified according to any of the claims 1-3,
2) 2D-PAGE immunoblotting on whole cell lysates of cells infected with said bacteria using antibodies obtained in step 1),
3) detecting protein spots reacting in step 2),
4) identifying the proteins in the spots selected in step 3).

5. A method for identifying proteins secreted from an intracellular bacteria, comprising combinations of the methods according to claims 1 to 4.

6. A method according to any of the claims 1-5, wherein said labelling is by radioactive means, such as [35S]cysteine, [35S]methionine, [14C]labelled amino acids or combinations thereof.

7. A method according to any of the claims 1-6 for identifying proteins, which proteins either in their full length or as immunogenic fragments thereof, are suitable for inclusion in immunogenic compositions and/or diagnostic purposes.

8. A method according to any of the claims 1-7, wherein the identification method is based on Edman degradation or any mass spectrometric method, such as MALDI TOF MS (Matrix-Assisted Laser Desorption/Ionisation Time-Of-Flight Mass Spectrometry), ESI Q-TOF MS (Electrospray Ionisation Quadrupole Time-Of-Flight Mass Spectrometry), PSD-MALDI MS (Post Source Decay MALDI Mass Spectrometry) or combinations of such methods.

9. A method according to any of the claims 1-8, wherein the proteins prior to identification are subjected to cleavage by chemical methods, such as cyanogen bromide treatment or hydroxylamine treatment, or by enzymatic methods with any suitable enzymes, such as trypsin, slymotrypsin, chymotrypsin, or pepsin, or combinations thereof.

10. A method according to any of the claims 1-9, wherein the intracellular bacteria is a facultative intracellular or obligate intracellular bacterium.

11. A method according to claim 10, wherein the bacterium is from the genus Chlamydia, such as C. pneumoniae, C. trachomatis, C. psittaci or C. pecorum, including any specific serovar or strain of these.

12. A method according to claim 11, wherein the intracellular bacterium is Chlamydia trachomatis.

13. A method according to claim 11, wherein the intracellular bacterium is Chlamydia pneumoniae.

14. A method according to any of the claims 1-13, wherein the host cell is an immortalized cell line, such as HeLa, Hep2, McCoy or U937, a primary cell line obtained from mammalian donors or by autopsy, a genetically modified cell line, or an organ cell culture.

15. A method according to claims 14, wherein the host cells have been genetically modified to over-express or suppress genes which are recognized as being relevant in context of chlamydial vaccine development, such as genes encoding proteasome subunits or other genes encoding functionally important proteins involved in MHC class presentation.

16. A method according to any of the claims 1-15, wherein the host cells are treated with IFN-γ prior to or during infection with the intracellular bacteria.

17. A method according to any of the claims 2 or 4-16, wherein proteasome inhibitors, such as MG132, MG262, MG115, epoxymycin, PSI and clasto-Lactacystin-β-lactone, or combinations thereof, are used.

18. A protein identifiable by the method of any of the claims 1-17 or an immunogenic fragment thereof.

19. A protein according to claim 18, which is applicable for inclusion in immunogenic compositions and/or diagnostic purposes, or an immunogenic fragment thereof.

20. A protein according to claim 19, which comprises T-cell epitopes being candidates for presentation as MHC-class I or II restricted antigens suitable for inclusion in immunogenic compositions.

21. A protein according to claim 20, which comprises T-cell epitopes being candidates for presentation as MHC-class I restricted antigens suitable for inclusion in immunogenic compositions.

22. A Chlamydia trachomatis protein according to any of the claims 18-21, having the pI and Mw characteristics of one of the proteins DT1-DT77 as given in Table I, determined with an average error of +/−10%, or an immunogenic fragment thereof.

23. A Chlamydia trachomatis protein according to any of the claims 18-21, which is identified by the corresponding gene number as CT017 (gene name CT017), CT044 (gene name ssp), CT243 (gene name IpxD), CT263 (gene name CT263), CT265 (gene name accA), CT286 (gene name clpC), CT292 (gene name dut), CT407 (gene name dksA), CT446 (gene name euo), CT460 (gene name SWIB), CT541 (gene name mip), CT610 (gene name CT610), CT650 (gene name recA), CT655 (gene name kdsA), CT668 (gene name CT668), CT691 (gene name CT691), CT734 (gene name CT734), CT783 (gene name CT783), CT858 (gene name CT858), CT875 (gene name CT875), or ORF5 (gene name ORF5), or by the gene name DT8 as given in Table IIIA, or an immunogenic fragment thereof.

24. A Chlamydia trachomatis protein according to any of the claims 18-21, having the pI and Mw characteristics of one of the proteins DT1, DT2, DT3, DT5, DT9, DT10, DT11, DT13, DT14, DT17, DT47, DT59, DT60, DT61 or DT62 as given in Table IV, determined with an average error of +/−10%, or an immunogenic fragment thereof.

25. A Chlamydia trachomatis protein according to claim 22, selected from the proteins DT4 (gene name CT858), DT23 (gene name mip), DT 47, DT48 (gene name CT858), DT75, DT76 (gene name CT691), and DT77 (gene name CT263), or an immunogenic fragment thereof.

26. A Chlamydia pneumoniae protein according to any of the claims 18-21, having the pI and Mw characteristics of one of the proteins CP1-CP91 as given in Table II, determined with an average error of +/−10%, or an immunogenic fragment thereof.

27. A Chlamydia pneumoniae protein according to any of the claims 18-21, which is identified by the corresponding gene number as CPN0152 (gene name CPN0152), CPN0702, CPN0705 (gene name CPN0705), CPN0711 (gene name CPN0711), CPN0796 (gene name CPN0796), CPN0998 (gene name ftsH), CPN0104 (gene name CPN0104), CPN0495 (gene name aspC), CPN0684 (gene name parB), CPN0414 (gene name accA), CPN1016 (gene name CPN1016), CPN1040 (gene name CPN1040), CPN0079 (gene name rl10), CPN0534 (gene name dksA), CPN0619 (gene name ndk), CPN0711 (gene name CPN0711), CPN0628 (gene name rs13), CPN0926 (gene name CPN0926), CPN1016 (gene name CPN1016) CPN1063 (gene name tpiS), or CPN0302 (gene name IpxD) as given in Table IIIB, or an immunogenic fragment thereof.

28. A Chlamydia pneumoniae protein according to claim 26, selected from the proteins CP34 (gene name CPN1016), CP37 (gene name CPN0998), CP46 (gene name CPN0796), CP47 (gene name CPN0705), CP52 (gene name CPN0152), CP63 (gene name CPN1016), and CP75 (gene name ndk), or an immunogenic fragment thereof.

29. A Chlamydia trachomatis polypeptide, characterized in that it is DT8 and comprises the following sequence (SEQ ID NO: 1):
MQHTIMLSLENDNDKLASMMDRVVAASSSILSASKDSESN
RQFTISKAPDKEAPCRVSYVAASALSE
or an immunogenic fragment thereof.

30. A protein having at least 40% sequence identity, preferably at least 60%, more preferably at least 70%, even more preferable at least 80%, further more preferable 90%, and most preferably at least 95% sequence identity to the proteins according to any of the claims 18-29, or an immunogenic fragment thereof.

31. A protein or an immunogenic fragment thereof, which comprises at least 7 consecutive amino acids of the proteins according to any of the claims 18-30.

32. A Chlamydia trachomatis protein or an immunogenic fragment thereof according to claim 31, which comprises an amino acid sequence, selected from the sequences of SEQ ID NO. 3-SEQ ID NO. 45.

33. A Chlamydia pneumoniae homolog of the Chlamydia trachomatis proteins according to claim 32 or an immunogenic fragment thereof, which comprises an amino acid sequence, selected from the sequences of SEQ ID NO. 122-SEQ ID NO. 148.

34. A Chlamydia pneumoniae protein or an immunogenic fragment thereof according to claim 31, which comprises an amino acid sequence, selected from the sequences of SEQ ID NO. 46-SEQ ID NO. 121.

35. A Chlamydia trachomatis homolog of the Chlamydia pneumoniae proteins according to claim 34 or an immunogenic fragment thereof, which comprises an amino acid sequence, selected from the sequences of SEQ ID NO. 149-SEQ ID NO. 194.

36. A nucleic acid compound, which comprises a sequence that encodes a protein, or an immunogenic fragment thereof, according to any of the claims 18-35.

37. A nucleic acid compound, which comprises a sequence that encodes a polypeptide of claim 29.

38. A nucleic acid compound according to claim 37, which comprises the following sequence (SEQ ID NO: 2):
ATGCAACACACAATTATGCTGTCTTTAGAGAACGATAATGATAAGCTTGC
TTCTATGATGGATCGAGTTGTTGCTGCGTCATCAAGCATTCTTTCTGCTT
CCAAAGATTCTGAGTCCAATAGACAGTTTACTATTTCTAAAGCTCCGGAT
AAAGAAGCTCCTTGCAGAGTATCTTATGTAGCTGCAAGTGCACTTTCAGA
ATAG
or a fragment or degenerative sequence thereof.

39. A vector comprising a nucleic acid compound according to any of the claims 36-38.

40. A host cell transformed or transfected with a vector according to claim 39.

41. Use of a protein or an immunogenic fragment thereof according to any of the claims 18-35 for the production of antibodies against said protein or fragment.

42. A method for producing an antibody against intracellular bacteria, wherein a protein or an immunogenic fragment thereof according to any of the claims 18-35 are administered to a producing animal, and the antibody is purified there from.

43. An antibody obtainable by the method according to claim 42.

44. A pharmaceutical or diagnostic composition comprising a protein or fragment thereof, according to any of the claims 18-35, an antibody according to claim 43 or a nucleic acid compound according to any of the claims 36-38.

45. Use of a protein or a fragment thereof according to any of the claims 18-35, an antibody according to claim 43 or a nucleic acid compound according to any of the claims 36-38 in the preparation of a diagnostic reagent.

46. A method for identification of T-cell epitopes on secreted proteins from intracellular bacterias, comprising steps, such as computer prediction, MHC class molecule binding assays and/or ELISPOT assays on a protein or an immunogenic fragment thereof identified in a method according to any of the claims 1-17

47. A peptide epitope obtainable by the method according to claim 46, which peptide epitope is likely to be surface presente

48. A peptide epitope comprising 4 to 25 consecutive amino acids of a protein according to any of the claims 18-31, preferably 6 to 15 amino acids, and most preferably 7 to 10 amino acids.

49. A peptide epitope comprising 7 to 10 consecutive amino acids of a Chlamydia trachomatis or Chlamydia p

50. A peptide epitope comprising 4 to 25 consecutive amino acids of a polypeptide comprising the sequence SEQ ID NO:1, preferably 6 to amino acids, and most preferably 7 to 10 amino acids.

51. A Chlamydia trachomatis peptide epitope according to claims 47, which comprises an amino acid sequence, selected from the sequences of SEQ ID NO. 3-SEQ ID NO. 45.

52. A Chlamydia pneumoniae peptide epitope of the Chlamydia trachomatis peptide epitopes of claim 51, which comprises an amino acid sequence, selected from the sequences of SEQ ID NO. 122-SEQ ID NO. 148.

53. A Chlamydia pneumonia peptide epitope according to claims 47, which comprises an amino acid sequence selected from the sequences of SEQ ID NO. 46-SEQ ID NO. 121.

54. A Chlamydia trachomatis peptide epitope of the Chlamydia pneumoniae peptide epitopes of claim 53, which comprises an amino acid sequence, selected from the sequences of SEQ ID NO. 149-SEQ ID NO. 194.

55. A peptide epitope according to any of the claims 47-54, characterized in that it is part of a fusion protein.

56. A peptide epitope according to any of the claims 47-54, characterized in that it is conjugated to a carrier moiety.

57. A nucleic acid compound, characterized in that it comprises a sequence that encodes a peptide epitope according to any of the claims 47-56.

58. A vector comprising a nucleic acid compound according to claim 57.

59. A host cell transformed or transfected with a vector according to claim 58.

60. Use of a peptide epitope of any of the claims 47-56 for the preparation of an immunogenic composition.

61. An immunogenic composition comprising a peptide epitope according to any of the claims 47-56, which immunogenic composition optionally contains a pharmaceutically acceptable excipient.

62. Use of a protein according to any of the claims 18-35, an antibody according to claim 43, a nucleic acid compound according to any of claims 36-38 or 57, or a peptide epitope according to any of claims 47-56 in the preparation of a pharmaceutical composition for treating or preventing infection due to an intracellular bacteria.

63. Use of a protein according to any of the claims 22-35, an antibody according to claim 43, a nucleic acid compound according to any of claims 36-38 or 57, or a peptide epitope according to any of claims 47-56 in the preparation of a pharmaceutical composition for treating or preventing infection due to a Chlamydia.

64. Use of a protein according to any of the claims 18-35, an antibody according to claim 43, a nucleic acid compound according to any of claims 36-38 or 57, or a peptide epitope according to any of claims 47-56 in the preparation of a diagnostic reagent for detecting the presence of an intracellular bacteria or antibodies raised against the intracellular bacteria.

65. Use of a protein according to any of the claims 22-35, an antibody according to claim 43, a nucleic acid compound according to any of claims 36-38 or 57, or a peptide epitope according to any of claims 47-56 in the preparation of a diagnostic reagent for detecting the presence of Chlamydia or antibodies raised against Chlamydia.

66. A method of inducing an immune response in a human, which comprises administering to said human an immunological effective amount of a protein according to any of claims 18-35, an antibody according to claim 43, a nucleic acid compound according to any of claims 36-38 or 57, or a peptide epitope according to any of claims 47-56.

67. A method according to claim 66 for treating or preventing infection of humans or animals by an intracellular bacteria.

68. A method according to any of the claims 66-67, wherein the intracellular bacteria is from the genus Chlamydia.

69. A method according to claim 68, wherein the intracellular bacteria is C. trachomatis.

70. A method according to claim 68, wherein the intracellular bacteria is C. pneumonia.

71. A method of producing a protein or a fragment thereof according to any of the claims 18-35, which comprises transforming, transfecting of infecting a host cell with a vector according to claim 39 and culturing the host cell under conditions, which permit the expression of said protein or fragment by the host cell.

72. A method of producing a peptide epitope of any any of the claims 47-54, which comprises transforming, transfecting of infecting a host cell with a vector according to claim 58 and culturing the host cell under conditions, which permit the expression of said peptide epitope by the host cell.
